(19) United States
(12) Patent Application Publication
(10) Pub. No.: US 2019/0002846 A1
(43) Pub. Date: Jan. 3, 2019

(54) POLYPEPTIDES HAVING DEMETHYLATING ACTIVITY

(71) Applicant: Genencor International B.V., Leiden (NL)

(72) Inventors: Martijn Johannes Koetsier, Wageningen (NL); Jacob Visser, Wageningen (NL); Svetlana Laura Iancu, Wageningen (NL); Mirjam Anna Kabel, Wageningen (NL); Matthias Frommhagen, Rhenen (NL); Harm Gruppen, Wageningen (NL); Heiko Lange, Roma (IT); Claudia Crestini, Roma (IT); Bouchra Benjelloun-Mlayah, Pompertuzat (FR)

(21) Appl. No.: 15/737,814

(22) PCT Filed: Jun. 24, 2016
(86) PCT No.: PCT/EP2016/064666

§ 371 Date: Dec. 19, 2017

(30) Foreign Application Priority Data
Jun. 24, 2015 (NL) 2015020

(51) Int. Cl. C12N 9/02; C12P 7/22

(52) U.S. Cl. CPC C12N 9/0061; C12P 7/22; C12Y 110/03002

(57) ABSTRACT

The present invention relates to fungal enzymes, more in particular of polyphenoloxidases (PPOs), and is based on a newly discovered enzymatic activity of a class of PPOs, i.e. de-methylation of R-substituted mono- or di-methoxyphenolsuch as present in lignin, lignin derived compounds and/or in lignocellulosic biomass. This newly discovered enzymatic activity renders these enzymes highly suitable for a plethora of applications in industry. Provided herein are methods of demethylation, processes to increase the reactivity of lignin or lignin-comprising biomass, processes of conversion lignin or lignin comprising biomass to value added products, processes for degrading and/or modifying (hemi-)cellulose in a hemicellulose-comprising substrate, and expression vectors, host cells and liquids, pastes or solid formulations and compositions for use in the demethylation method of the invention.

### CROSS-REFERENCE TO RELATED APPLICATIONS

This application claims the benefit of priority to Netherlands Patent Application No. NL2015020, filed on 24 Jun. 2015, the entirety of which is herein incorporated by reference.

### FIELD OF THE INVENTION

The present disclosure relates to the area of fungal enzymes, more in particular of polyphenoloxidases (PPOs). The disclosure is based on a newly discovered class of PPOs, based on their enzymatic activity, i.e. de-methylation of R-substituted mono- or di-methoxyphenols such as present in lignin, lignin derived compounds and/or in lignocellulosic biomass. This newly discovered enzymatic activity renders these enzymes highly suitable for a plethora of new applications in industry.

### BACKGROUND OF THE INVENTION

Lignin is a natural biopolymer, which can be extracted from biomass e.g. wood. Lignin is obtainable as a by-product of the paper and pulp industry and biorefinery industries and from a variety of low-value agricultural commodities such as straws, corn stover and bagasse. In biorefineries lignocellulosic biomass is pretreated using e.g. dilute sulfuric or other acids, hot water (hydrothermal), high pH (alkaline) by adding for example calcium-, sodium- or potassium hydroxide, or ammonia to breakdown the lignocellulose and producing a lignin fraction as a coproduct. For example lignin is coproduced as sulfite-, sulfonate-, kraft-, soda-organosolv (e.g. Pan et al., Biotechnol. Bioeng. 2005 May 20; 90(4):473-81.), or biolignin (WO2009/092749).

Potential high-value products from isolated lignin include low-cost carbon fibre, plastics and thermoplastic elastomers and fuels or chemicals currently derived from petroleum. For example lignin is considered to be a possible substitute for phenol for example in phenol-formaldehyde (PF) resin synthesis.

The low reactivity of lignin due to its chemical structure is preventing large scale commercial utilization of lignin. Demethylation increases the reactivity of lignin by forming catechol moieties in the lignin macromolecule (Hu et al. BIORESOURCES, Vol. 6 (3), 3515-3525: 2011).

Lignin is an aromatic heteropolymer consisting of the three monolignols (or hydroxycinnamyl alcohols) coniferyl (guaiacyl or “G” unit when incorporated in lignin polymer), sinapyl (syringyl or “S” unit when incorporated in lignin polymer) and p-coumaryl alcohol (p-hydroxyphenyl H unit when incorporated in lignin polymer), which are methoxylated in various degrees (Vanholme et al, Plant Physiology, July 2010, Vol. 153, pp. 895-905, 2012).

The lignin structure is depended on the type of plant source and the lignin isolation procedure. For example the ratio of syringyl (S), guaiacyl (G) and hydroxyphenyl (H) groups in the lignin is different in trees, grasses and straws. Especially the reactivity of lignins that are rich in S-groups and to a lesser extent G-groups (mono- and di-orthophenols) will be enhanced by demethylation.

It is known that lignin or lignin derived materials may be treated to cleave the methoxy groups (referred is to U.S. Pat. No. 2,816,832, U.S. Pat. No. 2,840,614, U.S. Pat. No. 2,908,716, U.S. Pat. No. 3,326,980 and U.S. Pat. No. 4,250,088). Each of these methods demethylate lignin to some extent, however these methods operate at high temperature and/or pressure, making them energy intensive and may result in modification of the lignin structure. It is therefore desirable to identify milder demethylation methods.

An alternative for a thermochemical demethylation is a biological demethylation. Several biological systems have been described to demethylate lignin and/or lignin-derived compounds. These biological systems rely for example on the activity of laccases, tetrahydrofo late-dependent transferases, or a dioxygenases to catalyze lignin demethylation. A laccase catalyzed demethylation has the drawback that this is not very specific, whereas the tetrahydrofo late-dependent transferase, and dioxygenase described for Sphingomonas paucimobilis SYK-6 strain are intracellular enzymes making isolation of the enzymes laborious and expensive. It would be desirable to obtain new methods for the demethylation.

### BRIEF SUMMARY OF THE INVENTION

Provided herein are polyphenoloxidases (PPOs) capable of demethylating an R-substituted mono- or di-methoxyphenol represented by

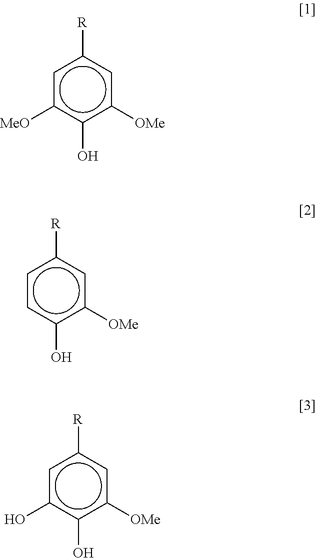

the above formula [1], [2] or [3] wherein R can be any single atom or chemical moiety. R may be an organic moiety. Preferably these PPOs comprise or consist of an amino acid sequence that is at least 70% identical to the amino acid sequence of at least one of SEQ ID NO: 37, 41 or 45. Preferably, the PPO is an enzyme that comprises a central tyrosinase domain. The PPO may be obtainable from a fungus, preferably a Myceliophthora. The PPO may be obtainable from Myceliophthora thermophila C1. Preferably, the PPO is an enzyme that releases methanol from an R-substituted di-methoxyphenol represented by

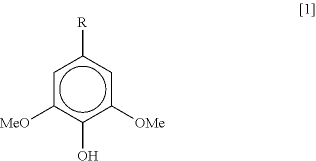

, wherein R is an organic moiety. Preferably, the R-substituted mono- or di-methoxyphenol is an R-substituted di-methoxyphenol represented by formula [1].

Provided herein are methods of demethylation of an R-substituted mono- or di-methoxyphenol represented by formula [1], [2] or [3] wherein R can be any single atom or chemical moiety, comprising contacting a substrate comprising said R-substituted mono- or di-methoxyphenol with the a polyphenoloxidase (PPO) capable of demethylating said R-substituted mono- or di-methoxyphenol as provided herein. The R-substituted mono- or di-methoxyphenol-comprising substrate may further comprise or be contacted with a copper salt. The copper salt may be selected from the group consisting of CuSO4, CuCl₂, CuBr₂, CuF₂, Cu(OH)₂ and Cu(NO₃)₂. In embodiments, the concentration of copper salt is 1-1000 μM. In embodiments, the R-substituted mono- or di-methoxyphenol-comprising substrate has a pH between 5.0 and 8.0. In embodiments, the method is performed at a temperature between 20-60° C. In embodiments, the R-substituted mono- or di-methoxyphenol is syringic acid, sinapic acid, syringol, and/or a combination thereof. Lignin may be the source of the R-substituted mono- or di-methoxyphenol in the demethylation method provided herein.

Provided herein are processes to increase the reactivity of lignin or lignin-comprising biomass, wherein said process comprises a method of demethylation provided herein. Also provided herein are processes for the conversion of lignin or lignin comprising biomass to products such as bio fuel, macromolecules and/or aromatic chemicals, wherein said process comprises a method of demethylation provided herein.

Provided herein are processes for degrading and/or modifying cellulose in a hemicellulose-comprising substrate, wherein said process comprises the step of contacting said substrate with an accessory enzyme and a PPO capable of demethylating an R-substituted mono- or di-methoxyphenol represented by formula [1], [2], or [3] wherein R can be any single atom or chemical moiety. In embodiments, the process comprises the step of admixing an R-substituted mono- or di-methoxyphenol represented by formula [1], [2], or [3] wherein R can be any single atom or chemical moiety. In embodiments, the accessory enzyme is an lytic polysaccharide monoxoygenases (LPMO) that comprises or consists of an amino acid sequence that is at least 70% identical to the amino acid sequence of SEQ ID NO: 1 or SEQ ID NO: 93. In embodiments, the process is a process for saccharification, for degrading DDG, for baking, for preparing dough, for clarifying fruit, vegetable or cereal juice, for macerating vegetables or food and/or for increasing digestibility and/or nutritional properties of animal feedstocks or animal food.

Provided herein are expression vectors encoding a PPO capable of demethylating a compound of formula [1], [2], or [3] wherein R can be any single atom or chemical moiety. In embodiments, the PPO comprises or consists of an amino acid sequence that is at least 70% identical to the amino acid sequence of SEQ ID NO: 37, 41 or 45. In embodiments, expression vectors may further encode an LPMO that is at least 70% identical to the amino acid sequence of SEQ ID NO: 1 or SEQ ID NO: 93.

Also provided herein are host cells transformed with expression vectors provided herein.

Provided herein are liquid, paste or solid formulations for use in methods of demethylation provided herein, wherein said formulation comprises a PPO provided, and preferably further comprising one or more additional enzymes. In embodiments, the formulation further comprises an LPMO that is at least 70% identical to the amino acid sequence of SEQ ID NO: 1 or SEQ ID NO: 93

Provided herein are compositions comprising an R-substituted mono- or di-methoxyphenol-comprising substrate, wherein said composition further comprises a PPO disclosed herein, an expression vector disclosed herein, a host cell disclosed herein, a liquid, paste or solid formulation disclosed herein and/or a combination thereof. In embodiments, the composition is a biomass saccharification mixture, a baking composition or dough, animal feed or a feed premix, lignocellulosic material, paper and/or pulp.

Provided herein are uses of a PPO a PPO disclosed herein, an expression vector disclosed herein, a host cell disclosed herein, a liquid, paste or solid formulation disclosed herein, and/or a composition provided herein, in a method or process.

### BRIEF DESCRIPTION OF THE DRAWINGS

FIG. 1: Proposed PPO7 catalyzed reaction with syringic acid. The enzyme of the present invention catalyzes the demethylation of dimethoxy substituted phenol compounds (ortho substituents). The enzyme also catalyzes the subsequent oxidation of the catechol to the quinone.

FIG. 2: Difference absorbance spectra (syringic acid blank subtracted) of the reaction of syringic acid with purified PPO7 and PPO2 after 24 h.

FIG. 3: Visual color change upon incubation of syringic acid with purified PPO7, PPO2 and blank as described in Example.

FIG. 4: UV-VIS data of the reactions of crude PPOs with synaptic acid (FIG. 4a), syringic acid (FIG. 4b), and syringol (FIG. 4c). Enzyme blanks were subtracted.

FIG. 5: UHPLC (FIG. 5a), MS (FIG. 5b) and MS² (FIG. 5c) data for the PPO reaction with syringic acid. Data confirm the formation of 3,4-dihydroxy-5-methoxybenzoic acid.

### DETAILED DESCRIPTION

In order to further define this invention, the following terms and definitions are herein provided. All publications, patents and other references mentioned herein are incorporated by reference in their entireties for all purposes.

Described herein is the discovery of PPO catalyzed demethylation of lignin, lignin derived compounds and other R-substituted mono- or di-methoxyphenols as defined herein. To our knowledge a (tyrosinase-like) PPO has not previously been found to catalyze this activity. As used herein, the terms “lignin”, “lignen”, “saccharification”, “fermentable sugars”, “biomass”, “lignocellulosic material”, “agricultural biomass”, “energy crops”, “cellulose” and “hemicellulose” are to be understood herein as defined in WO2013/159005 A2. As used herein, “oxidoreductase”, “oxidase”, “monooxygenases”, “hydroxylases” “dehydrogenases”, “cellobiose dehydrogenase”, “cellobiose dehydrogenases”, “cellobiose oxidases”, “carbohydrase”, “glycoside hydrolase”, “glycosyl hydrolase”, “glycosidase”, “endoglucanase”, “cellobiohydrolase” and “hemicellulase”, “xylanase”, “β-mannanase”, “endo-1,4-β-mannosidase”, “mannan endo-1,6-α-mannosidase”, “β-mannosidase”, “galactanase”, “endo-β-1,6-galactanse”, “arabino galactan endo-1,4-β-galactosidase”, “glucoamylase” “β-hexosaminidase”, “β-N-acetylglucosaminidase”, “α-L-arabinofuranosidase”, “α-N-arabinofuranosidase”, “α-arabinofuranosidase”, “arabino sidase”, “arabinofuranosidase”, “endo-arabinase”, “exo-arabinase”, “β-xylosidase”, “chitosanase”, “exo-polygalacturonase”, “acetyl xylan esterase”, “acetyl mannan esterase”, “ferulic esterase”, “ferulic acid esterase”, “coumaric acid esterase”, “pectate lyase”, “pectin lyases”, “endo-1,3-β-glucanase”, “laminarinase”, “lichenase”, “glycosidases” and “ligninase” are to be understood herein as defined in WO 2013/159005 A2.

As used herein, a “biomass” is understood herein as defined in WO2013/159005 A2, i.e. including materials containing cellulose and/or hemicellulose. Generally, these materials also contain pectin, lignin, protein, carbohydrates (such as starch and sugar) and ash. Lignocellulose is generally found, for example, in the stems, leaves, hulls, husks, and cobs of plants or leaves, branches, and wood of trees. Biomass can include virgin biomass and/or non-virgin biomass such as agricultural biomass, commercial organics, construction and demolition debris, municipal solid waste, waste paper and yard waste. Common forms of biomass include trees, shrubs and grasses, wheat, wheat straw, sugar cane bagasse, sugar beet, soybean, corn, corn husks, corn kernel including fiber from kernels, products and by-products from milling of grains such as corn, tobacco, wheat and barley (including wet milling and dry milling) as well as municipal solid waste, waste paper and yard waste. The biomass can also be, but is not limited to, herbaceous material, agricultural residues, forestry residues, municipal solid wastes, waste paper, and pulp and paper mill residues. “Agricultural biomass” includes branches, bushes, canes, corn and corn husks, energy crops, algae, fruits, flowers, grains, grasses, herbaceous crops, leaves, bark, needles, logs, roots, saplings, short rotation woody crops, shrubs, switch grasses, trees, vegetables, fruit peels, vines, sugar beet pulp, wheat midlings, oat hulls, peat moss, mushroom compost and hard and soft woods (not including woods with deleterious materials). In addition, agricultural biomass includes organic waste materials generated from agricultural processes including farming and forestry activities, specifically including forestry wood waste. Agricultural biomass may be any of the aforestated singularly or in any combination or mixture thereof “Agricultural biomass” includes branches, bushes, canes, corn and corn husks, energy crops, algae, fruits, flowers, grains, grasses, herbaceous crops, leaves, bark, needles, logs, roots, saplings, short rotation woody crops, shrubs, switch grasses, trees, vegetables, fruit peels, vines, sugar beet pulp, wheat midlings, oat hulls, peat moss, mushroom compost and hard and soft woods (not including woods with deleterious materials). In addition, agricultural biomass includes organic waste materials generated from agricultural processes including farming and forestry activities, specifically including forestry wood waste. Agricultural biomass may be any of the aforestated singularly or in any combination or mixture thereof.

As used herein, an “organic moiety” is an H or O atom, an —OH group or a branched, straight or cyclic alkyl which is optionally substituted and which can vary from a single atom to a large, optionally branched, structure.

As used herein, reference to an “enzyme” includes full-length naturally occurring or wild-type enzymes or proteins (the terms enzyme and protein are used herein interchangeably) and their glycosylated or otherwise modified forms, fusion proteins, or any fragment or homologue or variant of such an enzyme or protein. Preferably, an enzyme is an isolated enzyme or protein. An isolated enzyme or protein, is to be understood herein as an enzyme or protein (including a polypeptide or peptide) that has been removed from its natural milieu (i.e., that has been subject to human manipulation) and can include purified proteins, partially purified proteins, recombinantly produced proteins, synthetically produced proteins, proteins complexed with lipids, soluble proteins, and isolated proteins associated with other proteins, for example. As such, “isolated” does not reflect the extent to which the protein has been purified. Preferably, an isolated protein is produced recombinantly. In addition, and by way of example, a “M. thermophila protein” or “M. thermophila enzyme” refers to a protein (generally including a homologue or variant of a naturally occurring protein) from Myceliophthora thermophila or to a protein that has been otherwise produced from the knowledge of the structure (e.g., sequence) and preferably the function of a naturally occurring protein from Myceliophthora thermophila. In other words, a M. thermophila protein includes any protein that has substantially similar structure and function of a naturally occurring M. thermophila protein or that is a biologically active (i.e., has biological activity) homologue or variant of a naturally occurring protein from M. thermophila as described in detail herein. As such, a M. thermophila protein can include purified, partially purified, recombinant, mutated/modified and synthetic proteins.

As used herein, the phrase “biological activity” of a protein refers to any function(s) exhibited or performed by the protein that is ascribed to the naturally occurring form of the protein as measured or observed in vitro or in vivo. A protein fragment preferably comprises a domain of a protein that has the catalytic activity of the full-length enzyme. A protein fragment includes, but is not limited to, a fragment comprising a catalytic domain (CD) and/or a carbohydrate binding module (CBM). For example, a protein fragment may comprise a CD of a protein but not a CBM of the protein or a CBM of a protein but not a CD. Similarly, domains from different proteins may be combined. Protein fragments comprising a CD, CBM or combinations thereof for each protein disclosed herein can be readily produced using standard techniques known in the art.

An enzyme or protein, including a biologically active homologue, variant, or fragment thereof, has at least one characteristic of biological activity of a wild-type, or naturally occurring, protein. As discussed above, in general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). The biological activity of a protein as disclosed herein can include an enzyme activity (catalytic activity and/or substrate binding activity), such as oxidases, oxygenases, monoxygenases, Baeyer-Villiger monooxygenases, dioxygenases, peroxidases, dehydrogenases, reductases that catalyze an oxidation-reduction reaction or any other activity disclosed herein. Specific biological activities of the proteins disclosed herein are described in detail herein and in the Examples. Methods of detecting and measuring the biological activity of a protein as disclosed herein include, but are not limited to, the assays described in the Examples section below. Such assays include, but are not limited to, measurement of enzyme activity (e.g., catalytic activity), measurement of substrate binding, and the like. Additional assays and methods for examining the activity of the enzymes are found in U.S. Patent Applications 60/806,876, 60/970,876, 11/487,547, 11/775,777, 11/833,133, and 12/205,694 and U.S. Patent Application Publications US20080076159A1, US20090099079A1, US20070238155A1, US20090280105A1 and incorporated herein by reference.

It is noted that an enzyme or protein (including homologues or variants) is not required to have a biological activity such as catalytic activity. A protein can be a truncated, mutated or inactive protein, or lack at least one activity of the wild-type enzyme, for example. Inactive proteins may be useful in some screening assays. Methods to measure protein expression levels of a protein include, but are not limited to: western blotting, immunocytochemistry, flow cytometry or other immunologic-based assays; assays based on a property of the protein including but not limited to, ligand binding or interaction with other protein partners.

Modifications of a protein, such as in a homologue or variant, may result in proteins having the same biological activity as the naturally occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications that result in an increase in protein expression or an increase in the activity of the protein, can be referred to as amplification, overproduction, activation, enhancement, up-regulation or increased action of a protein.

As used herein, the terms “modification,” “mutation,” and “variant” can be used interchangeably with regard to the modifications/mutations to the amino acid sequence of a M. thermophila protein (or nucleotide sequences) described herein.

The term “modification” can also be used to describe post-translational modifications to a protein or peptide including, but not limited to, methylation, farnesylation, carboxymethylation, geranyl geranylation, glycosylation, phosphorylation, acetylation, myristoylation, prenylation, palmitation, and/or amidation. Modification can also include the cleavage of a signal peptide, or methionine, or other portions of the peptide that require cleavage to generate the mature peptide.

As used herein, the terms “homologue” or “variants” are used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide), insertions and/or substitutions; changes in stereochemistry of one or a few atoms; and/or minor derivatizations, including but not limited to for example: methylation, glycosylation and phosphorylation. A homologue or variant can have either enhanced, decreased, or substantially similar properties as compared to the naturally occurring protein or peptide. A homologue or variant can include an agonist of a protein or an antagonist of a protein.

Homologues or variants can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Homologous can also be the result of a gene duplication and rearrangement, resulting in a different location. Allelic variants typically encode proteins having similar activity to that of the protein encoded by the gene to which they are being compared. One class of allelic variants can encode the same protein but have different nucleotide sequences due to the degeneracy of the genetic code. Allelic variants can also comprise alterations in the 5′ or 3′ untranslated regions of the gene (e.g., in regulatory control regions). Allelic variants are well known to those skilled in the art.

Homologues or variants can be produced using techniques known in the art for the production of proteins including, but not limited to, direct modifications to the naturally occurring protein, direct protein synthesis, or modifications to the nucleotide sequence encoding the protein using, for example, classic or recombinant DNA techniques to effect random or targeted mutagenesis.

A modified protein also includes a fusion protein that includes a domain of a protein as disclosed herein (including a homologue or variant) attached to one or more fusion segments, which are typically heterologous in sequence to the protein sequence (i.e., different than protein sequence). Suitable fusion segments in a modified protein include, but are not limited to, segments that can: enhance a protein's stability; provide other desirable biological activity; and/or assist with the purification of the protein (e.g., by affinity chromatography). A suitable fusion segment can be a domain of any size that has the desired function (e.g., imparts increased stability, solubility, action or biological activity; and/or simplifies purification of a protein). Fusion segments can be joined to amino and/or carboxyl termini of the domain of a protein as disclosed herein and can be susceptible to cleavage in order to enable straight-forward recovery of the protein. Fusion proteins are preferably produced by culturing a recombinant cell transfected with a fusion nucleic acid molecule that encodes a protein including the fusion segment attached to either the carboxyl and/or amino terminal end of a domain of a protein as disclosed herein. Accordingly, proteins as disclosed herein also include expression products of gene fusions (for example, used to overexpress soluble, active forms of the recombinant protein), of mutagenized genes (such as genes having codon modifications to enhance gene transcription and translation), and of truncated genes (such as genes having membrane binding modules removed to generate soluble forms of a membrane protein, or genes having signal sequences removed which are poorly tolerated in a particular recombinant host).

A modified protein also includes a protein as disclosed that has been modified by conservative amino acid substitution. “Conservative amino acid substitutions” refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

A protein or polypeptide defined as “consisting essentially of” a specified amino acid sequence, is an amino acid sequences described herein produced from at least one, and up to about 20, additional heterologous amino acids flanking each of the C- and/or N-terminal ends of the specified amino acid sequence. Heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleotide sequence encoding the specified amino acid sequence as it occurs in the gene, if such nucleotides in the naturally occurring sequence were translated using standard codon usage for the organism from which the given amino acid sequence is derived. In addition, and solely by way of example, a protein referenced as being derived from is derived from a particular organism, such as a fungus as defined herein above.

Many of the enzymes and proteins disclosed herein may be desirable targets for modification and use in the processes described herein. These proteins have been described in terms of function and amino acid sequence (and nucleotide sequence encoding the same) of representative wild-type proteins. Homologues or variants of a protein encompassed preferably comprise, consist essentially of, or consist of, an amino acid sequence that is at least 35%, 45%, 55%, 65%, 70%, 75%, 80%, 90%, identical, and more preferably at least 95% identical, 96%, 97%, identical, and most preferably at least about 99% identical, or any percent identity between 35% and 99%, in whole integers (i.e., 36%, 37%, etc.), to an amino acid sequence disclosed herein that represents the amino acid sequence of an enzyme or protein disclosed herein (including a biologically active domain of a full-length protein). Preferably, the amino acid sequence of the homologue or variant has a biological activity of the wild-type or reference protein or of a biologically active domain thereof (e.g., a catalytic domain). When denoting mutation positions, the amino acid position of the wild-type is typically used. The wild-type can also be referred to as the “parent”. Additionally, any generation before the variant at issue can be a parent.

The minimum size of a protein and/or homologue, variant or fragment is a size sufficient to have biological activity or, when the protein is not required to have such activity, sufficient to be useful for another purpose associated with a protein as disclosed herein, such as for the production of antibodies that bind to a naturally occurring protein. Preferably, the protein disclosed herein is at least 20, 25, 30, 40, 50, 60, 70, 80, 90, 100, 125, 150, 175, 200, 250 amino acids in length, and so on up to a full length of each protein, and including any size in between in increments of one whole integer (one amino acid). There is no limit, other than a practical limit, on the maximum size of such a protein in that the protein can include a portion of a protein or a full-length protein, plus additional sequence (e.g., a fusion protein sequence), if desired.

As used herein, unless otherwise specified, reference to a percent (%) identity refers to an evaluation of homology which is performed using: 1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402); 2) a BLAST 2 alignment (using the parameters described below); 3) PSI-BLAST with the standard default parameters (Position-Specific Iterated BLAST; and/or 4) CAZy homology determined using standard default parameters from the Carbohydrate Active EnZymes database (Coutinho, P. M. & Henrissat, B. (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12) and/or applying a similar strategy using databases such as the Foly database (website: foly.esil.univ-mrs.fr) and the PeroxiBase (website: peroxibase.isb-sib.ch).

It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues or variants. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:

Reward for match=1

Penalty for mismatch=−2

Open gap (5) and extension gap (2) penalties

gap x_dropoff (50) expect (10) word size (11) filter (on)

For blastp, using 0 BLOSUM62 matrix:

Open gap (11) and extension gap (1) penalties

gap x_dropoff (50) expect (10) word size (3) filter (on).

Unless otherwise indicated herein, identity with a given SEQ ID NO means identity based on the full length of said sequence (i.e. over its whole length or as a whole).

As used herein, the term “contiguous” or “consecutive”, with regard to nucleic acid or amino acid sequences described herein, means to be connected in an unbroken sequence. For example, for a first sequence to comprise 30 contiguous (or consecutive) amino acids of a second sequence, means that the first sequence includes an unbroken sequence of 30 amino acid residues that is 100% identical to an unbroken sequence of 30 amino acid residues in the second sequence. Similarly, for a first sequence to have “100% identity” or being “100% identical” with a second sequence means that the first sequence exactly matches the second sequence with no gaps between nucleotides or amino acids. A protein as disclosed herein, including a homologue or variant, preferably includes a protein having an amino acid sequence that is sufficiently similar to a natural amino acid sequence that a nucleotide sequence encoding the homologue or variant is capable of hybridizing under moderate, high or very high stringency conditions (described below) to (i.e., with) a nucleic acid molecule encoding the natural protein (i.e., to the complement of the nucleic acid strand encoding the natural amino acid sequence). Preferably, a homologue or variant of a protein is encoded by a nucleic acid molecule comprising a nucleotide sequence that hybridizes under low, moderate, or high stringency conditions to the complement of a nucleotide sequence that encodes a protein comprising, consisting essentially of, or consisting of, an amino acid sequence represented by any one of SEQ ID NOs 1-6, 10, 13, 14, 17, 18, 21, 22, 25, 26, 29, 30, 33, 34, 37, 38, 41, 42, 45 and 46. Such hybridization conditions are described in detail below.

A nucleotide sequence complement of nucleotide sequence encoding a protein as disclosed herein refers to the nucleotide sequence of the nucleic acid strand that is complementary to the strand, which encodes the protein. It will be appreciated that a double stranded DNA which encodes a given amino acid sequence comprises a single strand DNA and its complementary strand having a sequence that is a complement to the single strand DNA. As such, nucleic acid molecules can be either double-stranded or single-stranded, and include those nucleic acid molecules that form stable hybrids under stringent hybridization conditions with a nucleotide sequence that encodes an amino acid sequence such as the amino acid sequences of SEQ ID NOs 1-6, 10, 13, 14, 17, 18, 21, 22, 25, 26, 29, 30, 33, 34, 37, 38, 41, 42, 45 or 46 or a nucleotide sequence of SEQ ID NOs 7-9, 11, 12, 15, 16, 19, 20, 23, 24, 27, 28, 31, 32, 35, 36, 39, 40, 43, 44, 47 or 48. Methods to deduce a complementary sequence are known to those skilled in the art. It should be noted that since nucleic acid sequencing technologies are not entirely error-free, the sequences presented herein, at best, represent apparent sequences of the proteins disclosed herein.

As used herein, reference to hybridization conditions refers to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid.

More particularly, moderate stringency hybridization and washing conditions, as referred to herein, refer to conditions which permit isolation of nucleic acid molecules having at least about 70% nucleotide sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleotide sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleotide sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. Preferably, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. Preferably, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature (Tm) for molecules larger than about 100 nucleotides, 0% formamide and a G+C content of about 40%. Alternatively, Tm can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tm of a particular hybrid, and wash conditions typically include a combination of salt and temperature conditions that are approximately 12-20° C. below the calculated Tm of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

A nucleic acid molecule includes a nucleic acid molecule comprising, consisting essentially of, or consisting of, a nucleotide sequence encoding any of the enzymes or proteins disclosed herein, including a fragment or a homologue or variant of such proteins, described above. Nucleic acid molecules can include a nucleotide sequence that encodes a fragment of a protein that does not have biological activity, and can also include portions of a gene or polynucleotide encoding the protein that are not part of the coding region for the protein (e.g., introns or regulatory regions of a gene encoding the protein). Nucleic acid molecules can include a nucleotide sequence that is useful as a probe or primer (oligonucleotide sequences). Preferably, a nucleic acid molecule is an isolated nucleic acid molecule. As used herein, an isolated nucleic acid molecule is a nucleic acid molecule (polynucleotide) that has been removed from its natural milieu (i.e., that has been subject to human manipulation) and can include DNA, RNA, or derivatives of either DNA or RNA, including cDNA. As such, “isolated” does not reflect the extent to which the nucleic acid molecule has been purified. Although the phrase “nucleic acid molecule” primarily refers to the physical nucleic acid molecule, and the phrase “nucleotide sequence” primarily refers to the sequence of nucleotides on the nucleic acid molecule, the two phrases can be used interchangeably, especially with respect to a nucleic acid molecule, or a nucleotide sequence, being capable of encoding a protein. An isolated nucleic acid molecule can be isolated from its natural source or produced using recombinant DNA technology (e.g., polymerase chain reaction (PCR) amplification, cloning) or chemical synthesis. Nucleic acid molecules or isolated nucleic acid molecules can include, for example, genes, natural allelic variants of genes, coding regions or portions thereof, and coding and/or regulatory regions modified by nucleotide insertions, deletions, substitutions, and/or inversions in a manner such that the modifications do not substantially interfere with the nucleic acid molecule's ability to encode a protein or to form stable hybrids under stringent conditions with natural gene isolates. A nucleic acid molecule can include degeneracies. As used herein, nucleotide degeneracy refers to the phenomenon that one amino acid can be encoded by different nucleotide codons. Thus, the nucleotide sequence of a nucleic acid molecule that encodes a protein disclosed herein can vary due to degeneracies. It is noted that a nucleic acid molecule disclosed herein is not required to encode a protein having protein activity. A nucleic acid molecule can encode a truncated, mutated or inactive protein, for example. In addition, nucleic acid molecules disclosed herein are useful as probes and primers for the identification, isolation and/or purification of other nucleic acid molecules. If the nucleic acid molecule is an oligonucleotide, such as a probe or primer, the oligonucleotide preferably ranges from about 5 to about 50 or about 500 nucleotides, more preferably from about 10 to about 40 nucleotides, and most preferably from about 15 to about 40 nucleotides in length.

Reference to a gene includes all nucleotide sequences related to a natural (i.e. wild-type) gene, such as regulatory regions that control production of the protein encoded by that gene (such as, but not limited to, transcription, translation or post-translation control regions) as well as the coding region itself. A gene may be a naturally occurring allelic variant that includes a similar but not identical sequence to the nucleotide sequence encoding a given protein. Allelic variants have been previously described above. Genes can include or exclude one or more introns or any portions thereof or any other sequences or which are not included in the cDNA for that protein. The phrases “nucleic acid molecule” and “gene” can be used interchangeably when the nucleic acid molecule comprises a gene as described above.

Modified genes include natural genes modified by substitution, insertion, and/or deletion of single or multiple nucleotide sequences, which can occur within the coding sequence including exons of regions encoding a polypeptide, or in flanking regions, such as regulatory regions typically upstream (e.g., promoters, enhancers, and related sequences), downstream (e.g., transcriptional termination, and poly(A) signals), or internal regions (e.g., introns) that affect the transcription, translation, and/or activation of a polypeptide or regulatory molecule of interest. Activation of a polypeptide, for example, may require removal of one or more N-terminal, C-terminal, or internal polypeptide regions, and/or post-translational modification of specific amino acid residues, such as by glycosylation, amidation, etc., that may alter the targeting, degradation, catalytic activity, of an enzyme.

Preferably, a nucleic acid molecule as disclosed herein is produced using recombinant DNA technology (e.g., polymerase chain reaction (PCR) amplification, cloning, etc.) or chemical synthesis. Nucleic acid molecules include any nucleic acid molecules and homologues or variants thereof that are part of a gene described herein and/or that encode a protein described herein, including, but not limited to, natural allelic variants and modified nucleic acid molecules (homologues or variants) in which nucleotides have been inserted, deleted, substituted, and/or inverted in such a manner that such modifications provide the desired effect on protein biological activity or on the activity of the nucleic acid molecule. Allelic variants and protein homologues or variants (e.g., proteins encoded by nucleic acid homologues or variants) have been discussed in detail above.

A nucleic acid molecule homologue or variant (i.e., encoding a homologue or variant of a protein disclosed herein) can be produced using a number of methods known to those skilled in the art (see, for example, Sambrook et al.). For example, nucleic acid molecules can be modified using a variety of techniques including, but not limited to, by classic mutagenesis and recombinant DNA techniques (e.g., site-directed mutagenesis, chemical treatment, restriction enzyme cleavage, ligation of nucleic acid fragments and/or PCR amplification), or synthesis of oligonucleotide mixtures and ligation of mixture groups to “build” a mixture of nucleic acid molecules and combinations thereof. Another method for modifying a recombinant nucleic acid molecule encoding a protein is gene shuffling (i.e., molecular breeding) (See, for example, U.S. Pat. No. 5,605,793 to Stemmer; Minshull and Stemmer; 1999, Curr. Opin. Chem. Biol. 3:284-290; Stemmer, 1994, P.N.A.S. USA 91:10747-10751). This technique can be used to efficiently introduce multiple simultaneous changes in the protein. Nucleic acid molecule homologues or variants can be selected by hybridization with a gene or polynucleotide, or by screening for the function of a protein encoded by a nucleic acid molecule (i.e., biological activity).

The minimum size of a nucleic acid molecule as disclosed herein is a size sufficient to encode a protein (including a fragment, homologue, or variant of a full-length protein) having biological activity, sufficient to encode a protein comprising at least one epitope which binds to an antibody, or sufficient to form a probe or oligonucleotide primer that is capable of forming a stable hybrid with the complementary sequence of a nucleic acid molecule encoding a natural protein (e.g., under moderate, high, or high stringency conditions). As such, the size of the nucleic acid molecule encoding such a protein can be dependent on nucleic acid composition and percent homology or identity between the nucleic acid molecule and complementary sequence as well as upon hybridization conditions per se (e.g., temperature, salt concentration, and formamide concentration). The minimal size of a nucleic acid molecule that is used as an oligonucleotide primer or as a probe is typically at least about 12 to about 15 nucleotides in length if the nucleic acid molecules are GC-rich and at least about 15 to about 18 bases in length if they are AT-rich. There is no limit, other than a practical limit, on the maximal size of a nucleic acid molecule as disclosed herein, in that the nucleic acid molecule can include a portion of a protein encoding sequence, a nucleotide sequence encoding a full-length protein (including a gene), including any length fragment between about 20 nucleotides and the number of nucleotides that make up the full length cDNA encoding a protein, in whole integers (e.g., 20, 21, 22, 23, 24, 25 nucleotides), or multiple genes, or portions thereof. The phrase “consisting essentially of”, when used with reference to a nucleotide sequence herein, refers to a nucleotide sequence encoding a specified amino acid sequence that can be flanked by from at least one, and up to as many as about 60, additional heterologous nucleotides at each of the 5 and/or the 3′ end of the nucleotide sequence encoding the specified amino acid sequence. The heterologous nucleotides are not naturally found (i.e., not found in nature, in vivo) flanking the nucleotide sequence encoding the specified amino acid sequence as it occurs in the natural gene or do not encode a protein that imparts any additional function to the protein or changes the function of the protein having the specified amino acid sequence.

A nucleic acid molecule as disclosed herein may be a recombinant nucleic acid molecule which comprises the nucleic acid molecule described above which is operatively linked to at least one expression control sequence. More particularly, a recombinant nucleic acid molecule typically comprises a recombinant vector and any one or more of the nucleic acid molecules as described herein. As used herein, a recombinant vector is an engineered (i.e., artificially produced) nucleic acid molecule that is used as a tool for manipulating a nucleotide sequence of choice and/or for introducing such a nucleotide sequence into a host cell. The recombinant vector is therefore suitable for use in cloning, sequencing, and/or otherwise manipulating the nucleotide sequence of choice, such as by expressing and/or delivering the nucleotide sequence of choice into a host cell to form a recombinant cell. Such a vector typically contains nucleotide sequences that are not naturally found adjacent to nucleotide sequence to be cloned or delivered, although the vector can also contain regulatory nucleotide sequences (e.g., promoters, untranslated regions) which are naturally found adjacent to nucleotide sequences disclosed herein or which are useful for expression of the nucleic acid molecules disclosed herein (discussed in detail below). The vector can be either RNA or DNA, either prokaryotic or eukaryotic, and typically is a plasmid. The vector can be maintained as an extrachromosomal element (e.g., a plasmid) or it can be integrated into the chromosome of a recombinant host cell, although it is preferred if the vector remains separate from the genome. The entire vector can remain in place within a host cell, or under certain conditions, the plasmid DNA can be deleted, leaving behind the nucleic acid molecule disclosed herein. An integrated nucleic acid molecule can be under chromosomal promoter control, under native or plasmid promoter control, or under a combination of several promoter controls. Single or multiple copies of the nucleic acid molecule can be integrated into the chromosome. A recombinant vector disclosed herein can contain at least one selectable marker.

A recombinant vector used in a recombinant nucleic acid molecule disclosed herein may be an expression vector. As used herein, the phrase “expression vector” is used to refer to a vector that is suitable for production of an encoded product (e.g., a protein of interest, such as an enzyme as disclosed herein). A nucleotide sequence encoding the product to be produced (e.g., the protein or homologue or variant thereof) may be inserted into a recombinant vector to produce a recombinant nucleic acid molecule. The nucleotide sequence encoding the protein to be produced is inserted into the vector in a manner that operatively links the nucleotide sequence to regulatory sequences in the vector, which enable the transcription and translation of the nucleotide sequence within the recombinant host cell. Typically, a recombinant nucleic acid molecule includes at least one nucleic acid molecule as disclosed herein operatively linked to one or more expression control sequences (e.g., transcription control sequences or translation control sequences). As used herein, the phrase “recombinant molecule” or “recombinant nucleic acid molecule” primarily refers to a nucleic acid molecule or nucleotide sequence operatively linked to a transcription control sequence, but can be used interchangeably with the phrase “nucleic acid molecule”, when such nucleic acid molecule is a recombinant molecule as discussed herein. As used herein, the phrase “operatively linked” refers to linking a nucleic acid molecule to an expression control sequence in a manner such that the molecule can be expressed when transfected (i.e., transformed, transduced, transfected, conjugated or conduced) into a host cell. Transcription control sequences are sequences that control the initiation, elongation, or termination of transcription. Particularly important transcription control sequences are those that control transcription initiation, such as promoter, enhancer, operator and repressor sequences. Suitable transcription control sequences include any transcription control sequence that can function in a host cell or organism into which the recombinant nucleic acid molecule is to be introduced. Transcription control sequences may also include any combination of one or more of any of the foregoing.

Recombinant nucleic acid molecules can also contain additional regulatory sequences, such as translation regulatory sequences, origins of replication, and other regulatory sequences that are compatible with the recombinant cell. A recombinant molecule, including those that are integrated into the host cell chromosome, may also contain secretory signals (i.e., signal segment nucleotide sequences) to enable an expressed protein to be secreted from the cell that produces the protein. Suitable signal segments include a signal segment that is naturally associated with the protein to be expressed or any heterologous signal segment capable of directing the secretion of the protein as disclosed herein. A recombinant molecule may comprise a leader sequence to enable an expressed protein to be delivered to and inserted into the membrane of a host cell. Suitable leader sequences include a leader sequence that is naturally associated with the protein, or any heterologous leader sequence capable of directing the delivery and insertion of the protein to the membrane of a cell.

The term “transfection” is generally used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells or plants and describes an inherited change due to the acquisition of exogenous nucleic acids by the microorganism that is essentially synonymous with the term “transfection.” Transfection techniques include, but are not limited to, transformation, particle bombardment, electroporation, microinjection, lipofection, adsorption, infection and protoplast fusion.

A “xylo-oligomer” is defined herein as an oligomer that is produced after cleavage of the β-1,4 bond in the xylan backbone. Preferably, a xylo-oligmer is an, optionally substituted, β-(1→4)-linked xylan consisting of 2 to 50, or 2 to 30, or of 2 to 15, or 2 to 10, or of 2 to 7 xylan residues. A substituted xylo-oligomer may be, but is not limited to, an arabino-, an acetyl-, a glucorono- and/or a methyglucurono-substituted xylo-oligomer. An “oxidised xylo-oligomer” is a xylo-oligomer that is oxidised on the C1- and/or C4-carbon atom of the xylo-oligomer.

A “gluco-oligomer” is defined herein as an oligomer that is produced after cleavage of the β-1,4 bond in the cellulose backbone. Preferably, a gluco-oligmer is a β-(1→4)-linked glucan consisting of 2 to 50, or 2 to 30, or preferably of 2 to 15, or 2 to 10, or most preferably of 2 to 6 glucan residues. An “oxidised gluco-oligomer” is a gluco-oligomer that is oxidised on the C1- and/or C4-carbon atom of the gluco-oligomer.

The word “approximately” or “about” when used in association with a numerical value (approximately 10, about 10) preferably means that the value may be the given value of more or less 10% of the value.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

The present invention relates to a newly discovered enzyme activity, i.e. to the demethylation of an R-substituted mono- or di-methoxyphenol by polyphenoloxidase (PPO) enzymes, wherein said R-substituted mono- or di-methoxyphenol is represented by the formula [1], [2] or [3], preferably by formula [1] or [2], most preferably by formula [1]. Within each of the formulas [1], [2] and [3], R can be any single atom or chemical moiety, but preferably is an organic moiety as defined herein, preferably comprising or consisting of 1-1000 atoms, wherein preferably each atom is selected from the group consisting of C, N, H, O and S. R preferably is —H, —OH, —COOH, —CH═CHCOOH, —CH₂═CH₂CH₃OH or lignin mono-, di- or polymer. The R-substituted mono- or di-methoxyphenol represented by the formula [1], [2] or [3] is, for example, any one selected from the group consisting of coniferyl, sinapyl, syringic acid, sinapic acid, ferulic acid, vanillic acid, 3,4 dihydroxy-5-methoxybenzoic acid, 3,4-dihydroxy-5-methoxycinnamic acid, 2-methoxyphenol (guaiacol) and syringol. It will be appreciated that, combinations of such R-substituted mono- or di-methoxyphenol may be employed. R-substituted mono- or di-methoxyphenol is, for example, 3-hydroxy-4-methoxycinnamic acid, 4-hydroxy-3-methoxyphenylactone, coniferyl aldehyde, ferulic acid, guaiacol, hesperidin, homovanillic acid, vanillic acid, 4-allyl-2,4-methoxyphenol, 3,4-dihydroxybenzoic acid, 3-methylcatechol, caffeic acid, 3,4-dihydroxy-5-methoxybenzoic acid or 3,4-dihydroxy-5-methoxycinnamic acid. R-substituted mono-methoxyphenol may, for example, be 3-hydroxy-4-methoxycinnamic acid, 4-hydroxy-3-methoxyphenylactone, coniferyl aldehyde, ferulic acid, guaiacol, homovanillic acid, vanillic acid, or caffeic acid. R-substituted di-methoxyphenol may, for example, be 4-allyl-2,4-methoxyphenol. R-substituted di-methoxyphenol may, for example, be 3,4-dihydroxy-5-methoxybenzoic acid or 3,4-dihydroxy-5-methoxycinnamic acid. R-substituted mono- or di-methoxyphenol represented by formulas [1], [2] or [3], wherein R is defined as indicated above, is indicated herein further as an “R-substituted mono- or di-methoxyphenol”.

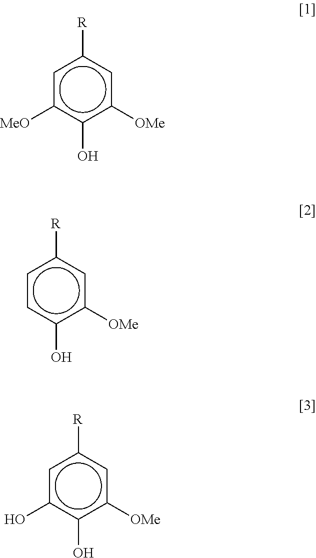

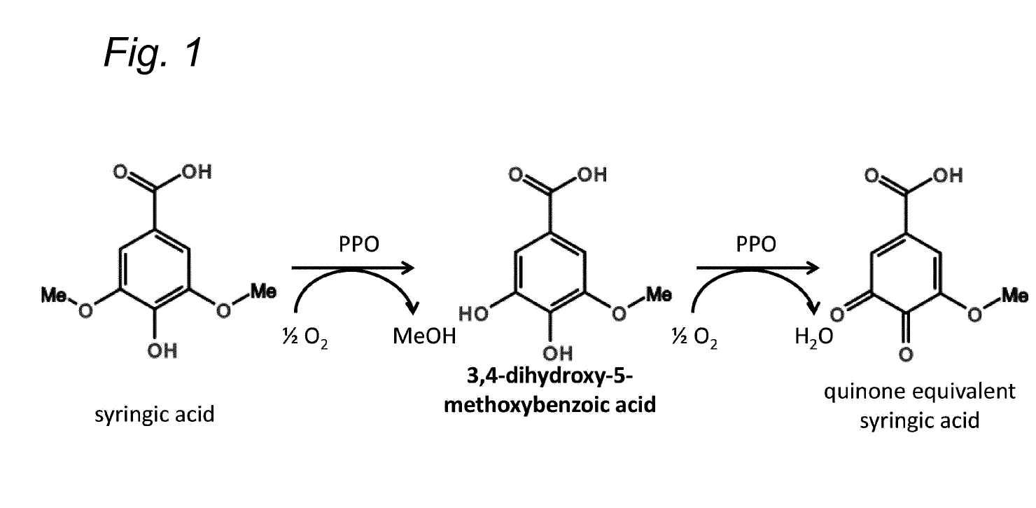

R-substituted mono- or di-methoxyphenols are present in lignin or lignin derived materials. The inventors discovered that the PPOs demethylate R-substituted di-methoxyphenols such as 4-hydroxy-3,5-dimethoxybenzoic acid (syringic acid) with the formation of 3,4-dihydroxy-5-methoxybenzoic acid and simultaneous release of methanol (see FIG. 1 and Examples). The mode of action of the PPO is important for improving the reactivity of lignin or lignin derived materials and enable valorization.

In a first aspect, the present invention provides an enzyme that belongs to a class of enzymes, i.e fungal tyrosinas-like polyphenoloxidases or PPOs, for which demethylation of R-substituted mono- or di-methoxyphenol compounds, such as dimethoxy phenol (syringol), 4-hydroxy-3,5-dimethoxybenzoic acid (syringic acid) and 3-(4-hydroxy-3,5-dimethoxyphenyl)prop-2-enoic acid (sinapic acid) has been established for the first time. Preferably, the enzyme of the invention comprises or consists of a common central tyrosinase domain, preferably of the family with NCBI accession number pfam00264 (Volbeda and Hol, J. Mol. Biol. Volume 209(2): pages 249-279, 1989). Preferably, the enzyme of the invention is an enzyme that comprises or consists of an amino acid sequence that is at least 50%, 60%, 70%, 80%, more preferably at least 90%, 91%, 92%, 93%, 94%, even more preferably at least 95%, 96%, 97%, 98%, 99% or at least 100% identical to any one of SEQ ID NO: 37 (PPO4), SEQID NO: 41 (PPO6) and SEQ ID NO: 45 (PPO7). SEQ ID NO: 37, 41 and 45 represent the mature form of PPO4, PPO6, and PPO7, respectively. SEQ ID NO: 38, 42 and 46 represent PPO4, PPO6 and PPO7 including the signal sequence, respectively. Preferably, the enzyme of the invention is an enzyme that comprises or consists of an amino acid sequence that is at least 50%, 60%, 70%, 80%, more preferably at least 90%, 91%, 92%, 93%, 94%, even more preferably at least 95%, 96%, 97%, 98%, 99% or at least 100% identical to any one of SEQ ID NO: 37 (PPO4), SEQ ID NO: 41 (PPO6) or SEQ ID NO: 45 (PPO7) and comprises a common central tyrosinase domain, preferably of the family with NCBI accession number pfam00264. Preferably said PPO is obtainable from a fungus, preferably of the genus Myceliophthora. Being “obtainable from” is to be understood herein as that the enzyme originates from the indicated organism, i.e. is naturally produced by the indicated organism as a native enzyme. More preferably, the PPO originates from M. thermophila C1 fungus or derivatives thereof, such as a M. thermophila C1 fungus selected from Garg 27K, (Accession No. VKM F-3500D), UV13-6 (Accession No. VKM F-3632 D); NG7C-19 (Accession No. VKM F-3633 D); UV18-25 (Accession No. VKM F-3631 D); strain W1L (Accession No. CBS122189) or W1L#100L (Accession No. CBS122190), preferably a M. thermophila C1 (Accession No. VKM F-3500D).

Preferably, the PPO of the invention, preferably for use in a method or process of the invention, has at least 50%, 60%, 70%, 80%, more preferably at least 90%, 91%, 92%, 93%, 94%, even more preferably at least 95%, 96%, 97%, 98%, 99% or at least 100% identical to any one of SEQ ID NO: 37 (PPO4), SEQ ID NO: 41 (PPO6) or SEQ ID NO: 45 (PPO7), is capable of demethylating an R-substituted mono- or di-methoxyphenol as defined herein. Being capable of demethylating an R-substituted mono- or di-methoxyphenol as defined herein, is to be understood herein as having at least 10%, 20%, 30%, 40%, 50%, 60%, 70%, 80%, 90%, 91%, 92%, 93%, 94%, 95%, 96%, 97%, 98%, 99% or 100%, preferably at least 40% of the demethylation activity of PPO7 as assessed by a method known in the art for assessing demethylation activity, preferably by using an assay as exemplified herein and when using a R-substituted mono- or di-methoxyphenol as substrate, preferably said R-substituted mono- or di-methoxyphenol being syringic acid. In brief, in said method, the coding sequence of the enzyme of the invention is cloned in a pChi vector which is subsequently transformed in the Myceliophthora thermophila C1 strain W1L#100.1Δpyr5ΔAlp1 using the pyr5 marker as examplified herein (Example 1). Fermentations are preformed and subsequently, the broth is harvested, filtrated to remove fungal biomass, concentrated and dialyzed against 20 mM KPi pH 7.0 plus 150 mM KCl using a 5 kDa PES membrane (Vivacell® 70, Sartorius) as exemplified herein (Example 2). Optionally, samples are subsequently freeze-dried for storage and/or purified as exemplified herein (Example 5). Demethylating activity of the crude or purified enzyme can be determined by measuring an increased negative absorbance between 230 nm and 300 nm of syringic acid solution incubated with the enzyme (at RT for 24H as compared to OH), preferably as exemplified herein (Example 6). An enzyme of the invention is understood herein to have at least 10% (or higher) of the demethylation activity of PPO7 if the enzyme shows an increase in negative absorbance that is at least this percentage (i.e. 10% or higher) of the increase found when expressing, producing and purifying PPO7 under the exact same conditions (i.e. using the coding sequence of PPO7, SEQ ID NO: 48).

Demethylating activity of the crude or purified enzyme can also be determined by measuring methanol production of syringic acid solution incubated with the enzyme (at RT for 90H) as exemplified herein (Example 8). An enzyme of the invention is understood to have at least 10% (or higher) of the demethylation activity of PPO7 if the enzyme shows an increase in methanol production that is at least this percentage (i.e. 10% or higher) of the increase found when expressing, producing and purifying PPO7 under the exact same conditions (i.e. using the coding sequence of PPO7, SEQ ID NO: 48).

Preferably, the enzyme of the invention has an optimal demethylation activity in the presence of a copper salt at a concentration of between about 1 and about 1000 μM, preferably between about 20 and about 1000 μM, wherein said copper salt is preferably selected from the group consisting of CuSO4, CuCl₂, CuBr₂, CuF₂, Cu(OH)₂ and Cu(NO₃)₂. Preferably the enzyme of the invention has an optimal demethylation activity at a pH between about 5.0 and about 8.0. Preferably the enzyme of the invention has an optimal demethylation activity at a temperature between about 20 and about 60° C. Preferably, the enzyme of the invention has demethylation activity in the presence of a copper salt at a concentration of between about 1 and about 1000 μM, preferably between about 20 and about 1000 μM, wherein said copper salt is preferably selected from the group consisting of CuSO4, CuCl₂, CuBr₂, CuF₂, Cu(OH)₂ and Cu(NO₃)₂, at a pH between about 5.0 and about 8.0 and at a temperature between about 20 and about 60° C.

Preferably, the enzyme of the invention is capable of demethylating the R-substituted mono- or di-methoxyphenol syringic acid. Preferably, the enzyme is capable of demethylating a substrate comprising R-substituted di-methoxyphenol (represented by formula I, wherein R is an organic moiety). More preferably, the enzyme of the invention is capable of demethylating the R-substituted mono- or di-methoxyphenol syringic acid, sinapic acid and syringol, preferably as assessed in a method as exemplified herein (Example 7). Preferably, the enzyme of the invention is capable of demethylating lignin and lignin-derived compounds from substrates such as wheat straw, corn stover, lowest molecular weight from lignin fractionation and carboxymodified lignin (wheat or corn), preferably as assessed as exemplified herein (Example 9). Preferably, the enzyme of the invention is not capable of demethylating 3-dimethoxybenzene and 3,5-dimethoxybenzoic acid, for example as assessed as exemplified herein (Example 9).

In a second aspect, the invention provides for a nucleic acid molecule comprising, consisting essentially of, or consisting of, a nucleotide sequence encoding any of the enzyme of the first aspect. As further detailed in the Definitions Section, the nucleic acid molecule of the invention may include portions of a gene or polynucleotide encoding the protein that are not part of the coding region for the protein (e.g., introns or regulatory regions of a gene encoding the protein) and may be double stranded, single stranded and can include DNA, RNA, or derivatives of either DNA or RNA, including cDNA, probes and primers. Preferably, said encoding nucleic acid sequence has at least 50%, 60%, 70%, 80%, or more preferably at least 90%, 91%, 92%, 93%, 94%, 95%, 96%, 97%, 98%, 99% or 100% identity with SEQ ID NO: 39 (cDNA encoding PPO4), 40 (genomic DNA “gDNA” encoding PPO4), SEQ ID NO: 43 (cDNA encoding PPO6), SEQ ID NO: 44 (gDNA encoding PPO6), SEQ ID NO: 47 (cDNA encoding PPO7) or SEQ ID NO: 48 (gDNA encoding PPO7). Preferably, said encoding nucleic acid sequence has at least 50%, 60%, 70%, 80%, or more preferably at least 90%, 91%, 92%, 93%, 94%, 95%, 96%, 97%, 98%, 99% or 100% identity with SEQ ID NO: 40 (gDNA encoding PPO4), 44 (gDNA encoding PPO6), or 48 (gDNA encoding PPO7). Preferably, the nucleic acid molecule is an isolated nucleic acid molecule. Preferably, within the nucleic acid molecule of the invention, the nucleic acid sequence encoding any of the enzyme of the first aspect is operably linked to at least one expression control sequence and is preferably part of a (recombinant) expression vector as further detailed in the Definitions Section and/or as specifically exemplified herein (Example 1).

Optionally, the nucleic acid molecule encodes for one or more further enzymes, preferably such as defined in the fourth aspect herein. The nucleic acid molecule may be an expression vector (chimeric vector) encoding at least two enzymes of any one of the multi-enzyme complexes as defined in the fourth aspect herein, preferably said at least two enzymes or the PPO enzyme of the invention and the AA9 LPMO or LPMO9B as defined herein and as defined in EP15158572 or WO2010/107303A2, respectively, both of which are incorporated herein by reference. Also encompassed is an expression vector (chimeric vector) that expresses at the PPO enzyme of the invention, the LPMO, and an enzyme encoding a cellulase, for example an endoglucanase. Preferably, within said chimeric vector, the sequence encoding said AA9 LPMO encodes for an LPMO enzyme that has at least 50%, 60%, 70%, 80%, more preferably at least 90%, 91%, 92%, 93%, 94%, even more preferably at least 95%, 96%, 97%, 98%, or at least 99% identical to SEQ ID NO: 1. Preferably, said nucleic acid molecule comprises or consists of a nucleotide sequence that is at least 50%, 60%, 70%, 80%, more preferably at least 90%, 91%, 92%, 93%, 94%, even more preferably at least 95%, 96%, 97%, 98%, 99% or 100% identical to SEQ ID NO: 7, wherein SEQ ID NO: 7 represents the cDNA encoding MtLPMO9A. Preferably, said nucleic acid molecule comprises or consists of a nucleotide sequence that is at least 50%, 60%, 70%, 80%, more preferably at least 90%, 91%, 92%, 93%, 94%, even more preferably at least 95%, 96%, 97%, 98%, 99% or 100% identical to SEQ ID NO: 8, wherein SEQ ID NO: 8 represents the gDNA encoding MtLPMO9A, wherein the gDNA is to be understood herein as encompassing intron sequences. Preferably, said nucleic acid molecule comprises or consists of a nucleotide sequence that is at least 50%, 60%, 70%, 80%, more preferably at least 90%, 91%, 92%, 93%, 94%, even more preferably at least 95%, 96%, 97%, 98%, 99% or 100% identical to SEQ ID NO: 91, wherein SEQ ID NO: 91 represents the gDNA encoding MtLPMO9B. Optionally, within said chimeric vector, the sequence encoding said endoglucanase encodes for an endoglucanase enzyme that has at least 50%, 60%, 70%, 80%, more preferably at least 90%, 91%, 92%, 93%, 94%, even more preferably at least 95%, 96%, 97%, 98%, 99% or at least 100% identical to SEQ ID NO: 3, SEQ ID NO: 10, SEQ ID NO: 14, SEQ ID NO: 18, SEQ ID NO: 22, SEQ ID NO: 26 or SEQ ID NO: 30. Preferably, said endoglucanase encoding sequence is at least 50%, 60%, 70%, 80%, more preferably at least 90%, 91%, 92%, 93%, 94%, even more preferably at least 95%, 96%, 97%, 98%, 99% or 100% identical to SEQ ID NO: 9, wherein SEQ ID NO: 9 represents the cDNA encoding TνEG I as defined herein. Preferably, said endoglucanase encoding sequence is at least 50%, 60%, 70%, 80%, more preferably at least 90%, 91%, 92%, 93%, 94%, even more preferably at least 95%, 96%, 97%, 98%, 99% or 100% identical to SEQ ID NO: 9 (cDNA encoding TνEG I), SEQ ID NO: 11 (cDNA encoding MtEG VIII), SEQ ID NO: 15 (cDNA encoding MtEG II), SEQ ID NO: 19 (cDNA encoding MtEG I), SEQ ID NO: 23 (cDNA encoding MtEG III), SEQ ID NO: 27 (cDNA encoding MtEG V), SEQ ID NO: 31 (cDNA encoding MtEG VI). Preferably, said endoglucanase encoding nucleotide sequence is at least 50%, 60%, 70%, 80%, more preferably at least 90%, 91%, 92%, 93%, 94%, even more preferably at least 95%, 96%, 97%, 98%, 99% or 100% identical to SEQ ID NO: 12 (gDNA encoding MtEG VIII), SEQ ID NO: 16 (gDNA encoding MtEG II), SEQ ID NO: 20 (gDNA encoding MtEG I), SEQ ID NO: 24 (gDNA encoding MtEG III), SEQ ID NO: 28 (gDNA encoding MtEG V), SEQ ID NO: 32 (gDNA encoding MtEG VI).

It will be appreciated by one skilled in the art that use of recombinant DNA technologies can improve control of expression of transfected nucleic acid molecules by manipulating, for example, the number of copies of the nucleic acid molecules within the host cell, the efficiency with which those nucleic acid molecules are transcribed, the efficiency with which the resultant transcripts are translated, and the efficiency of posttranslational modifications. Additionally, the promoter sequence might be genetically engineered to improve the level of expression as compared to the native promoter. Recombinant techniques useful for controlling the expression of nucleic acid molecules include, but are not limited to, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites), modification of nucleic acid molecules to correspond to the codon usage of the host cell, and deletion of sequences that destabilize transcripts.

In a third aspect, the invention provides for a host cell comprising a nucleic acid molecule of the second aspect. Preferably, said host cell is transfected with an expression vector of the second aspect and said host cell is capable of expressing the enzyme of the first aspect encoded by said expression vector. The host cell of the invention may be, but is not limited to, any bacterial, fungal (e.g., filamentous fungi or yeast or mushrooms), algae, plant, insect, or animal cell that can be transfected. Preferably the host cell is a cultured cell. The host cell (e.g., a host cell or production organism) may include any microorganism (e.g., a bacterium, a protist, an alga, a fungus, or other microbe), and is preferably a bacterium, a yeast or a filamentous fungus. Suitable bacterial genera include, but are not limited to, Escherichia, Bacillus, Lactobacillus, Pseudomonas and Streptomyces. Suitable bacterial species include, but are not limited to, Escherichia coli, Bacillus subtilis, Bacillus licheniformis, Bacillus stearothermophilus, Lactobacillus brevis, Pseudomonas aeruginosa and Streptomyces lividans. Suitable genera of yeast include, but are not limited to, Saccharomyces, Schizosaccharomyces, Candida, Hansenula, Pichia, Kluyveromyces, and Phaffia. Suitable yeast species include, but are not limited to, Saccharomyces cerevisiae, Schizosaccharomyces pombe, Candida albicans, Hansenula polymorpha, Pichia pastoris, Pichia canadensis, Kluyveromyces marxianus and Phaffia rhodozyma.

Suitable fungal genera include, but are not limited to, Chrysosporium, Thielavia, Neurospora, Aureobasidium, Filibasidium, Piromyces, Corynascus, Cryptococcus, Acremonium, Tolypocladium, Scytalidium, Schizophyllum, Sporotrichum, Penicillium, Gibberella, Myceliophthora, Mucor, Aspergillus, Fusarium, Humicola, and Trichoderma, Talaromyces, Rasamsonia and anamorphs and teleomorphs thereof. Suitable fungal species include, but are not limited to, Aspergillus niger, Aspergillus oryzae, Aspergillus nidulans, Aspergillus japonicus, Absidia coerulea, Rhizopus oryzae, Myceliophthora thermophila, Neurospora crassa, Neurospora intermedia, Trichoderma reesei, Trichoderma longibrachiatum, Penicillium canescens, Penicillium solitum, Penicillium funiculosum, Myceliophthora thermophila, Acremonium alabamense, Thielavia terrestris, Sporotrichum thermophile, Sporotrichum cellulophilum, Chaetomium globosum, Corynascus heterothallicus, Talaromyces emersonii, Rasamsonia emersonii and Talaromyces flavus.

The host cell of the invention may be a genetically modified cell or microorganism. Preferably, the host cell is modified/optimized for the production of the enzyme of the invention. Preferably, the host cell is modified in order to produce reduced amounts or lacks the production of enzymes that interfere/compete with the expression and/or secretion of the enzyme of the invention as defined in the first aspect herein and/or with the enzymatic activity of the method of the invention as defined in the sixth aspect herein. Such host cell may be modified in order to downregulate the interfering enzyme activity. The downregulation may be achieved, for example, by introduction of inhibitors (chemical or biological) of the enzyme activity, by manipulating the efficiency with which those nucleic acid molecules are transcribed, the efficiency with which the resultant transcripts are translated, and the efficiency of post-translational modifications, or by gene mutation, disruption or deletion, i.e. “knocking out” the endogenous copy of the gene. A “knock out” of a gene refers to a molecular biological technique by which the gene in the organism is made inoperative, so that the expression of the gene is substantially reduced or eliminated. Alternatively, the activity of the enzyme may be upregulated. Also contemplated herein is downregulating activity of one or more enzymes while simultaneously upregulating activity of one or more enzymes to achieve the desired outcome.

Preferably, a low cellulose strain is used. Preferably, the host cell is a fungal cell of strain C1 (VKM F-3500 D) or a mutant strain derived therefrom (e.g., UV13-6 (Accession No. VKM F-3632 D); NG7C-19 (Accession No. VKM F-3633 D); UV18-25 (VKM F-3631D), W1L (CBS122189), or W1L#100L (CBS122190)). Preferably, the host strain is a strain with reduced expression of protease and (hemi-)cellulase, more preferably free of protease and (hemi-)cellulase expression. Most preferred, the host strain is W1L#100.1Δpyr5ΔAlp1, also denominated as the LC strain (Visser H, et al., Ind. Biotechnol. 7:214-222, 2011 and WO2010/107303, which is incorporated herein by reference. As described in U.S. Pat. No. 6,015,707 or U.S. Pat. No. 6,573,086 a strain called C1 (Accession No. VKM F-3500 D), was isolated from samples of forest alkaline soil from Sola Lake, Far East of the Russian Federation. This strain was deposited at the All-Russian Collection of Microorganisms of Russian Academy of Sciences (VKM), (www.vkm.ru; Bakhurhina St. 8, Moscow, Russia, 113184; Prospekt Nauki No. 5, Pushchino, Moscow Region, 142290, Russia), under the terms of the Budapest Treaty on the International Regulation of the Deposit of Microorganisms for the Purposes of Patent Procedure on Aug. 29, 1996, (by A. P. Sinitsyn, O. N. Okunev, I. V. Solov'eva, V. M. Chernoglasov, M. A. Emalfarb, A. Ben-Bassat; “FermTech” LTD Acad. Kapitsky str. 32-2, Moscow, 117647, Russia) as Chrysosporium lucknowense Garg 27K, VKM F-3500 D. Various mutant strains of C1 have been produced and these strains have also been deposited at the All-Russian Collection of Microorganisms of Russian Academy of Sciences (VKM), (Bakhurhina St. 8, Moscow, Russia, 113184; Prospekt Nauki No. 5, Pushchino, Moscow Region, 142290, Russia), under the terms of the Budapest Treaty on the International Regulation of the Deposit of Microorganisms for the Purposes of Patent Procedure on Sep. 2, 1998 (by O. N. Okunev, A. P. Sinitsyn, V. M. Chernoglasov, and M. A. Emalfarb; “FermTech” LTD, Acad. Kapitsy str., 32-2, Moscow, 117647, Russia) or at the Centraal Bureau voor Schimmelcultures (CBS), Uppsalalaan 8, 3584 CT Utrecht, The Netherlands for the purposes of Patent Procedure on Dec. 5, 2007 (by Dyadic Nederland B. V., Nieuwe Kanaal 7s, 6709 PA Wageningen, Nederland). For example, Strain C1 was mutagenised by subjecting it to ultraviolet light to generate strain UV13-6 (Accession No. VKM F-3632 D; deposited with VKM on Sep. 2, 1998)). This strain was subsequently further mutated with N-methyl-N′-nitro-N-nitrosoguanidine to generate strain NG7C-19 (Accession No. VKM F-3633 D; deposited with VKM on Sep. 2, 1998). This latter strain in turn was subjected to mutation by ultraviolet light, resulting in strain UV18-25 (Accession No. VKM F-3631 D; deposited with VKM on Sep. 2, 1998). This strain in turn was again subjected to mutation by ultraviolet light, resulting in strain W1L (Accession No. CBS122189; deposited with CBS Dec. 5, 2007), which was subsequently subjected to mutation by ultraviolet light, resulting in strain W1L#100L (Accession No. CBS122190; deposited with CBS Dec. 5, 2007). Strain C1 was initially classified as a Chrysosporium lucknowense based on morphological and growth characteristics of the microorganism, as discussed in detail in U.S. Pat. No. 6,015,707, U.S. Pat. No. 6,573,086 and patent PCT/NL2010/000045. The C1 strain was subsequently reclassified as Myceliophthora thermophila based on genetic tests. C. luknowense has also appeared in the literature as Sporotrichum thermophile.

Also preferred is a genetically modified cell or microorganism, which is modified by having a reduction or elimination of enzymatic activities causing the formation of cellobionolactone/cellobionic acid and/or gluconolactone/gluconic acid. Enzymes causing the formation of cellobionolactone/cellobionic acid and/or gluconolactone/gluconic acid include. Preferably, such genetically modified cell or microorganism is a fungal strain lacking functional genes encoding enzymes causing the formation of cellobionolactone/cellobionic acid and/or gluconolactone/gluconic acid e.g., may be generated by gene deletion, gene disruption, gene silencing or mutation; or by deletion, disruption or mutation of gene expression regulatory sequences such as promoter sequences, terminator sequences, promoter activating sequences and sequences encoding transcription factors; or by random or site-directed mutation of the genes encoding the enzymes causing the formation of cellobionolactone/cellobionic acid and/or gluconolactone/gluconic acid. Preferably the modified fungus is a modified fungus wherein the one or more genes encoding enzymes responsible for the production of one or more products selected from cellobionolactone, cellobionic acid, gluconolactone, and gluconic acid encode a cellobiose dehydrogenase (CDH) is deleted, disrupted or mutated. Preferably, the one or more genes encode an enzyme having an amino acid sequence of the cellobiose dehydrogenase (CDH) selected from a group of polypeptides having at least 90%, 95%, or 99% homology with any of the polypeptides of SEQ ID NOS: 4-6. The modified fungus may be a modified fungus wherein the gene encoding the cellobiose dehydrogenase (CDH) CDH1 (SEQ ID NO: 4), CDH2 (SEQ ID NO: 5), or CDH3 (SEQ ID NO: 6) is deleted, disrupted or mutated. Preferably, the modified fungus is a fungus wherein CDH activity is reduced from about 50% to about 100%, or at least 75%, 90%, or 95%, when measured by a ferricyanide reduction assay. Preferably the gene encoding CDH1 (SEQ ID NO: 4) or encoding CDH2 (SEQ ID NO: 5) in Myceliophthora thermophila C1 is knocked out. More preferably, the genes encoding CDH1 and CDH2 in Myceliophthora thermophila C1 are both knocked out (double knock out), preferably as described in WO2013/159005A2 and WO2012/061432A1 which are incorporated herein by reference.

Further suitable host cells include insect cells (most particularly Drosophila melanogaster cells, Spodoptera frugiperda Sf9 and Sf21 cells and Trichoplusia High-Five cells), nematode cells (particularly C. elegans cells), avian cells, amphibian cells (particularly Xenopus laevis cells), reptilian cells, and mammalian cells (most particularly human, simian, canine, rodent, bovine, or sheep cells, e.g. NIH3T3, CHO (Chinese hamster ovary cell), COS, VERO, BHK, HEK, and other rodent or human cells).

The present invention also contemplates genetically modified organisms such as algae, bacterial, and plants transformed with one or more nucleic acid molecules of the second aspect herein. The plants may be used for production of the enzymes, and/or as the lignin, lignocellulosic, cellulosic and/or hemicellulosic material used as a substrate in a method of the invention. Methods to generate recombinant plants are known in the art. For instance, numerous methods for plant transformation have been developed, including biological and physical transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pp. 67-88. In addition, vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119.

The most widely utilized method for introducing an expression vector into plants is based on the natural transformation system of Agrobacterium. See, for example, Horsch et al., Science 227:1229 (1985). Descriptions of Agrobacterium vector systems and methods for Agrobacterium-mediated gene transfer are provided by numerous references, including Gruber et al., supra, Miki et al., supra, Moloney et al., Plant Cell Reports 8:238 (1989), and U.S. Pat. Nos. 4,940,838 and 5,464,763.

Another generally applicable method of plant transformation is microprojectile-mediated transformation, see e.g., Sanford et al., Part. Sci. Technol. 5:27 (1987), Sanford, J. C., Trends Biotech. 6:299 (1988), Sanford, J. C., Physiol. Plant 79:206 (1990), Klein et al., Biotechnology 10:268 (1992). Another method for physical delivery of DNA to plants is sonication of target cells. Zhang et al., Bio/Technology 9:996 (1991). Alternatively, liposome or spheroplast fusion have been used to introduce expression vectors into plants. Deshayes et al., EMBO J., 4:2731 (1985), Christou et al., Proc Natl. Acad. Sci. USA 84:3962 (1987). Direct uptake of DNA into protoplasts using CaCl₂) precipitation, polyvinyl alcohol or poly-L-ornithine have also been reported. Hain et al., Mol. Gen. Genet. 199:161 (1985) and Draper et al., Plant Cell Physiol. 23:451 (1982). Electroporation of protoplasts and whole cells and tissues have also been described. Donn et al., In Abstracts of VII^{th} International Congress on Plant Cell and Tissue Culture IAPTC, A2-38, p. 53 (1990); D'Halluin et al., Plant Cell 4:1495-1505 (1992) and Spencer et al., Plant Mol. Biol. 24:51-61 (1994).

In a fourth aspect, the invention provides for an enzyme composition or formulation comprising at least the enzyme of the first aspect.

The enzyme composition may be a crude fermentation product, preferably arising from recombinant microorganisms encoding the enzyme of the invention, or a preparation originating from a crude fermentation product that has been purified or partially purified using protein purification procedures known in the art. Alternatively, the enzyme of the enzyme composition is a protein or peptide that is produced synthetically (e.g., chemically, such as by peptide synthesis) or recombinantly in a substantial pure form.

The enzyme comprising formulation may be a cell-free composition or a composition comprising a host cell which comprises a nucleic acid molecule encoding the enzyme as defined herein, which is preferably capable of expressing the enzyme as defined herein.

The enzyme composition may be a multi-enzyme composition, which is understood to comprise the enzyme of the invention and at least one further (different) enzyme. A further enzyme is understood herein as an enzyme that is different in nature than the PPO enzyme of the invention and preferably has a different enzymatic activity. Specific multi-enzyme compositions as defined herein comprise the enzyme of the invention and at least one further enzyme that is particular suitable for use in a method of the invention as detailed herein below. The additional enzyme may also arise from an (optionally purified) crude fermentation product and/or be produced synthetically or recombinantly in a substantial pure form.

Preferably the enzyme or multi-enzyme composition is suitable for use in a method or process of the sixth aspect herein.

The enzyme or multi-enzyme composition of the invention may be a liquid, paste or solid formulation comprising the enzyme of the invention. Preferably, the solid formulation is a solid formulation that has the physical appearance of a granulate or a powder. A preferred formulation is a formulation comprising or consisting of coated or uncoated granules or micro-granules. The liquid, paste or solid formulation may be formulated in accordance with methods known in the art, preferably using methods known in the art of formulating enzymes and/or multi-enzyme compositions, feedstocks, chemical feedstocks, feed premixes, food supplements, bread improvers, flour treatment agents and/or pharmaceutical products. Preferably, the liquid, past or solid formulation according to this aspect is an enzyme or multi-enzyme composition, feedstock, chemical feedstock, animal feedstock, feed premix, food supplement, bread improver, flour treatment agent and/or pharmaceutical product, wherein preferably said composition comprises one or more further compounds known in the art to be suitable for use in such composition. Preferably the composition comprises stabilizers known in the art for enzyme stabilization, e.g. an antioxidant, a reducing agent, a polymer such as PVP, PVA, PEG or other suitable polymer known to be beneficial to the stability of polypeptides in solid or liquid compositions.

Preferably, granulates or agglomerated powder has a narrow particle size distribution with more than 95% (by weight) of the particles in the range from 10 to 2000 μm, preferably from 25 to 500 μm. Granulates and agglomerated powders may be prepared by conventional methods, e.g. by spraying the enzyme of the invention and optionally further enzymes onto a carrier in a fluid-bed granulator. The carrier may consist of particulate cores having a suitable particle size. The carrier may be soluble or insoluble, e.g. a salt (such as NaCl or sodium sulfate), a sugar (such as sucrose or lactose), a sugar alcohol (such as sorbitol), starch, rice, corn grits, or soy. Hence the invention also provides a granule comprising a composition comprising the enzyme of the invention, and optionally further enzymes, as defined herein.

It is to be understood that any of the enzymes described specifically herein can be combined with any one or more of the enzymes described herein or with any other available and suitable enzymes, to produce a multi-enzyme composition. The invention is not restricted or limited to the specific exemplary combinations listed below. One or more components of a multi-enzyme composition (other than the enzyme of the present invention) can be obtained from or derived from a microbial, plant, or other source or combination thereof.

The invention provides for a multi-enzyme composition, comprising at least the enzyme of the invention, that is for use in a method for conversion of lignin or a lignin-containing substrate to a value-added product, e.g. the production of biofuel, macromolecules and/or aromatic chemicals. Such multi-enzyme composition, preferably for use in a process to produce of macromolecules such as dispersants, emulsifiers, binders, sequestrants, carbon fibres, polymer modifiers, resins and adhesives, preferably further comprises one or more enzymes that specifically catalyze any one of sulfonation, introduction of ethyoxy, epoxy, vinyl and/or carbonyl moieties, depolymerization, intermolecular cross-linking, carbonylation, carboxylation, amination, epoxidation, de-etherification, oxidation and bioconjugation.

A further multi-enzyme composition, preferably for use in a process to produce aromatic monomers such as benzene, toluene, xylene, phenol, terephthalic acid monomeric lignin molecules, preferably further comprises enzymes that speficially catalyze dehyroxylation, dealkylation, hydrogenolysis, (selective) depolymerization, polymerization, sulfonation.

The multi-enzyme composition comprising at least the enzyme of the invention, may further comprise enzymes for degrading a lignocellulosic and/or hemicellulosic material or a biologically active fragment of an enzyme for degrading a lignocellulosic and/or hemicellulosic material. Such multi-enzyme composition is useful in particular for saccharification of (hemi-)cellulose in order to obtain fermentable product such as sugars. Apart from the enzyme of the invention, the multi-enzyme composition may further comprise at least one cellobiohydrolase, at least one xylanase, at least one endoglucanase, at least one β-glucosidase, at least one β-xylosidase, and at least one accessory enzyme. Preferably, the multi enzyme composition is free of enzymes which lead to the formation of cellobionolactone/cellobionic acid and/or gluconolactone/gluconic acid. A xylanase may be selected from the group consisting of: endoxylanases, exoxylanases, and β-xylosidases. Accessory enzymes include an enzyme selected from the group consisting of: cellulase, glucosidase, lytic polysaccharide monooxygenase (also referred to in the art as, for example, GH61 or polypeptide having cellulolytic enhancing activity), xylanase, xylosidase, ligninase, glucuronidase, arabinofuranosidase, arabinase, arabinogalactanase, ferulic acid esterase, lipase, pectinase, glucomannase, amylase, laminarinase, xyloglucanase, galactanase, galactosidase, glucoamylase, pectate lyase, chitosanase, exo-β-D-glucosaminidase, cellobiose dehydrogenase, and acetylxylan esterase. A preferred accessory enzyme is an LPMO of family AA9 (AA9 LPMO), preferably obtainable from Myceliophthora, preferably as defined in EP15158572 which is incorporated herein by reference, which is, more preferably obtainable from Myceliophthora thermophila C1. Preferably, the LPMO is an enzyme that comprises or consists of an amino acid sequence that is at least at least 50%, 60%, 70%, 80%, more preferably at least 90%, 91%, 92%, 93%, 94%, even more preferably at least 95%, 96%, 97%, 98%, 99% or at least 100% identical to SEQ ID NO: 1. The LPMO represented by SEQ ID NO: 1 is an AA9 family LPMO, denominated MtLPMO9A, originating from Myceliophthora thermophila C1 (Accession No. VKM F-3500D). The amino acid sequence of MtLPMO9A including the signal sequence is defined by SEQ ID NO: 2. A further preferred is accessory enzyme is an LPMO9B as defined herein. The multi-enzyme composition may further comprise at least one hemicellulase. A hemicellulase may be selected from the group consisting of a xylanase, an arabinofuranosidase, an acetyl xylan esterase, a glucuronidase, an endo-galactanase, a mannanase, an endo-arabinase, an exo-arabinase, an exo-galactanase, a ferulic acid esterase, a galactomannanase, a xyloglucanase, and mixtures thereof. A xylanase may be selected from the group consisting of endoxylanases, exoxylanases, and β-xylosidases in the absence of enzymes which lead to the formation of cellobionolactone/cellobionic acid and/or gluconolactone/gluconic acid. The multi-enzyme composition may further comprise at least one cellulase. The multi-enzyme composition may also include oxidases, oxygenases, monoxygenases, Baeyer-Villiger monooxygenases, dioxygenases, peroxidases, dehydrogenases, reductases that catalyze an oxidationreduction reaction. The multi-enzyme composition may further comprise at least one protein for degrading an arabinoxylan-containing material or a fragment thereof that has biological activity. The multi-enzyme composition may further comprise at least one endoxylanase, at least one β-xylosidase, and at least one arabinofuranosidase. An arabinofuranosidase may comprise an arabinofuranosidase with specificity towards single substituted xylose residues, an arabinofuranosidase with specificity towards double substituted xylose residues, or a combination thereof. The multi-enzyme compositions of a method of the invention can also include ligninases and lipases. While the multi-enzyme composition may contain many types of enzymes, mixtures comprising enzymes that increase or enhance sugar release from biomass, or biomass saccharification mixtures, are contemplated. Also preferred are mixtures that comprise enzymes that are capable of degrading cell walls and releasing cellular contents. Synergistic enzyme combinations and related methods are contemplated. The invention includes methods to identify the optimum ratios and compositions of enzymes. These methods entail tests to identify the optimum enzyme composition and ratios for efficient conversion of any biomass substrate to its constituent sugars. The Examples below include assays that may be used to identify optimum ratios and compositions of enzymes with which to degrade (ligno)cellulosic materials. Suitable enzymes may include, for example, enzymes disclosed in PCT Application Publication Nos. WO03/027306, WO200352118_A2, WO200352054_A2, WO200352057 A2, WO200352055_A2, WO200352056_A2, WO200416760_A2, WO9210581 A1, WO200448592_A2, WO200443980_A2, WO200528636_A2, WO200501065 A2, WO2005/001036, WO2005/093050, WO200593073 A1, WO200674005 A2, WO2009/149202, WO2011/038019, WO2010/141779, WO2011/063308, WO2012/125951, WO2012/125925, WO2012125937, WO/2011/153276, WO2014/093275, WO2014/070837, WO2014/070841, WO2014/070844, WO2014/093281, WO2014/093282, WO2014/093287, WO2014/093294, WO2015/084596, WO201517254 A1, W0201517255 A1, W0201517256 A1, or WO2016/069541, which are incorporated herein by reference.

The invention also provides for a multi-enzyme composition, comprising at least the enzyme of the invention, that is for use in a method for clarification and/or increasing the rate of filtration and/or decreasing haze formation of juices and/or beverages such as wine and beer. Such multi-enzyme composition preferably further comprises an LPMO (preferably the AA9 LPMO as defined herein above), pectinase, carboxymethylcellulase and/or amylase.

The invention also provides for a multi-enzyme composition, comprising at least the enzyme of the invention, that is for use in a method for macerating vegetables and/or fruit. Such multi-enzyme composition preferably further comprises an LPMO (preferably the AA9 LPMO or other LPMO as defined herein above), pectinase, carboxymethylcellulase and/or amylase.

The invention also provides for a multi-enzyme composition, comprising at least the enzyme of the invention, that is for use in a method for increasing digestibility and/or nutritional properties of animal feedstocks or animal food, such as, but not limited to, agricultural silage and grain feed. Such multi-enzyme composition preferably further comprises at least an LPMO (preferably the AA9 LPMO as defined herein above).

The invention also provides for a multi-enzyme composition, comprising at least the enzyme of the invention, that is for use in a method for increasing the quality of bakery products and/or improve textual characteristics (break and shred quality and crumb quality) of the bakery products such as, but not limited to bread, e.g. preferably for increasing the volume of the bakery product, e.g. a high-rising and/or light loaf of whole meal bread. Such multi-enzyme composition preferably further comprises an LPMO (preferably the AA9 LPMO as defined herein above), a cellulase, such as beta-glucanase, cellulase, cellobiohydrolase and/or beta-glucosidase. Preferably, the cellulase preparation is a complex of cellulases and/or hemicellulases, preferably obtained from Trichoderma sp., more preferably from T. reesei. Also preferred are cellulase preparations obtained from Aspergillus, preferably obtained from A. niger. A suitable cellulase composition may be CELLUCLAST™ (available from Novozymes) or Bakezyme® XU, Bakezyme® XE, BakeZyme® X-cell or Bakezyme® W (all available from DSM). The one or more additional enzymes for use in a method of the invention may further comprise additional enzymes, e.g., glucoamylase, α-amylase, xylanase, protease, lipase, phospholipase may be used together with the LPMO in the dough or the composition. The additional enzyme may be of any origin, including mammalian and plant, and preferably of microbial (bacterial, yeast or fungal) origin. The glucoamylase in this multi-enzyme composition may be a glucoamylase having a sequence identity of at least 50%, at least 60%, at least 65%, at least 70%, at least 75%, at least 80%, at least 85%, at least 90%, at least 95%, at least 96%, at least 97%, at least 98%, or at least 99% to the amino acid sequence of the A. niger G1 or G2 glucoamylase (Boel et al. (1984), EMBO J. 3(5), p. 1097-1102), the A. awamori glucoamylase disclosed in WO84/02921, or the A. oryzae glucoamylase (Agric. Biol. Chem. (1991), 55 (4), p. 941-949). The amylase may be fungal or bacterial, e.g. a maltogenic α-amylase from B. stearothermophilus or an α-amylase from Bacillus, e.g. B. licheniformis or B. amyloliquefaciens, a beta-amylase, e.g. from plant (e.g. soy bean) or from microbial sources (e.g. Bacillus), a glucoamylase, e.g. from A. niger, or a fungal α-amylase, e.g. from A. oryzae. Suitable commercial maltogenic α-amylases include NOVAMYLO (Novozymes A/S) and OPTICAKE® (Novozymes A/S). Suitable commercial fungal α-amylase compositions include, e.g., BAKEZYME P 300 (available from DSM) and FUNGAMYL 2500 SG, FUNGAMYL 4000 BG, FUNGAMYL 800 L, FUNGAMYL ULTRA BG and FUNGAMYL ULTRA SG (available from Novozymes A/S). The hemicellulase may be a pentosanase, e.g. a xylanase which may be of microbial origin, e.g. derived from a bacterium or fungus, such as a strain of Aspergillus, in particular of A. aculeatus, A. niger, A. awamori, or A. tubigensis, from a strain of Trichoderma, e.g. T. reesei, or from a strain of Humicola, e.g. H. insolens. Suitable commercially available xylanase preparations for use in the present invention include PENTOPAN MONO BG and PENTOPAN 500 BG (available from Novozymes), GRINDAMYL POWERBAKE (available from Danisco), and BAKEZYME BXP 5000 and BAKEZYME BXP 5001 (available from DSM). The protease may be from Bacillus, e.g. B. amyloliquefaciens. The lipase may be derived from a strain of Thermomyces (Humicola), Rhizomucor, Candida, Aspergillus, Rhizopus, or Pseudomonas, in particular from T. lanuginosus (H. lanuginosa), Rhizomucor miehei, C. antarctica, A. niger, R. delemar, R. arrhizus or P. cepacia. The phospholipase may have phospholipase A1, A2, B, C, D or lysophospholipase activity; it may or may not have lipase activity. It may be of animal origin, e.g. from pancreas, snake venom or bee venom, or it may be of microbial origin, e.g. from filamentous fungi, yeast or bacteria, such as Aspergillus or Fusarium, e.g. A. niger, A. oryzae or F. oxysporum. A preferred lipase/phospholipase from F. oxysporum is disclosed in WO 98/26057. Also, the variants described in WO 00/32758 may be used. The additional enzyme may be of any origin, including mammalian and plant, and preferably of microbial (bacterial, yeast or fungal) origin and may be obtained by techniques conventionally used in the art. Suitable phospholipase compositions are LIPOPAN F and LIPOPAN XTRA (available from Novozymes) or PANAMORE GOLDEN and PANAMORE SPRING (available from DSM).

The invention also provides for a multi-enzyme composition, comprising at least the enzyme of the invention, that is for use in the production of xylo-oligomers (XOs), or oxidised xylo-oligomers, which may be used in pharmaceutical, agriculture and feed products. XOs are known to have pre-biotic effects, as they are neither hydrolyzed nor absorbed in the upper gastrointestinal tract, and they affect the host by selectively stimulating the growth or activity of one or a number of bacteria in the colon, thus improving health. Furthermore, XOs are believed to reduce cholesterol levels, maintenance of gastrointestinal health, and improvement of the biological availability of calcium. Furthermore, XOs inhibit starch retrogradation, improving the nutritional and sensory properties of food. The LPMO as defined herein is highly suitable for XOs production because of the low exoxylanase and/or β-xylosidase activity. This is beneficial as exoxylanase and/or β-xylosidase activity results in production of xylose, which has inhibitory effects on XO production. Such multi-enzyme composition preferably further comprises at least an LPMO (preferably the AA9 LPMO as defined herein above).

A preferred multi-enzyme composition of the invention is any multi-enzyme composition as defined above that comprises the enzyme of the invention and an LPMO (preferably the AA9 LPMO as defined herein above). The inventors have found that the enzyme of the invention has a surprising effect on the LPMO enzymatic activity resulting in hemi-cellulose degradation/modification, on substrates that comprise (hemi-)cellulose and R-substituted mono- or di-methoxyphenols. Without being bound by any theory, this surprising effect may be due to the production of reducing agents due to the newly discovered demethylating activity of the enzyme of the invention.

Also preferred is a multi-enzyme composition of the invention that is any multi-enzyme composition as defined above that comprises the enzyme of the invention and an LPMO (preferably the AA9 LPMO as defined herein above), and optionally further comprising an endoglucanase, preferably an endoglucanase that has the amino acid sequence of SEQ ID NO: 3 (encoding TνEG I), SEQ ID NO: 10 (encoding MtEG VIII), SEQ ID NO: 14 (encoding MtEG II), SEQ ID NO: 18 (encoding MtEG I), SEQ ID NO: 22 (encoding MtEG III), SEQ ID NO: 26 (encoding MtEG V) or SEQ ID NO: 30 (encoding MtEG VI), and/or an endoglucanase as defined herein that has an amino acid sequence that is at least 50%, 60%, 70%, 80%, more preferably at least 90%, 91%, 92%, 93%, 94%, even more preferably at least 95%, 96%, 97%, 98%, or at least 99% identical to SEQ ID NO: 3, SEQ ID NO: 10, SEQ ID NO: 14, SEQ ID NO: 18, SEQ ID NO: 22, SEQ ID NO: 26 or SEQ ID NO: 30.

The enzymes of the multi-enzyme composition can be provided by a variety of sources. In one embodiment, the enzymes can be produced by growing organisms such as bacteria, algae, fungi, and plants which produce the enzymes naturally or by virtue of being genetically modified to express the enzyme or enzymes. In another embodiment, at least one enzyme of the multi-enzyme composition is a commercially available enzyme. Multi-enzyme compositions may be composed of enzymes from (1) commercial suppliers; (2) cloned genes expressing enzymes; (3) complex broth (such as that resulting from growth of a microbial strain in media, wherein the strains secrete proteins and enzymes into the media); (4) cell lysates of strains grown as in (3); and, (5) plant material expressing enzymes capable of degrading lignocellulose. In some embodiments, the accessory enzyme is a glucoamylase, a pectinase, or a ligninase.

The multi-enzyme compositions, in some embodiments, comprise microorganisms or a crude fermentation product of microorganisms. A crude fermentation product refers to the fermentation broth, which has been separated from the microorganism biomass (by filtration, for example). In general, the microorganisms are grown in fermenters, optionally centrifuged or filtered to remove biomass, and optionally concentrated, formulated, and dried to produce an enzyme(s) or a multi-enzyme composition that is a crude fermentation product. In other embodiments, enzyme(s) or multi-enzyme compositions produced by the microorganism (including a genetically modified microorganism as described herein) are subjected to one or more purification steps, such as ammonium sulfate precipitation, chromatography, and/or ultrafiltration, which result in a partially purified or purified enzyme(s). If the microorganism has been genetically modified to express the enzyme(s), the enzyme(s) will include recombinant enzymes. If the genetically modified microorganism also naturally expresses the enzyme(s) or other enzymes useful in a method of the invention, such as for lignocellulosic saccharification or any other useful application mentioned herein, the enzyme(s) may include both naturally occurring and recombinant enzymes.

Preferably, the multi-enzyme composition for use in a method of the invention comprising at least about 500 μg, and preferably at least about 1 μg, and more preferably at least about 5 μg, and more preferably at least about 10 μg, and more preferably at least about 25 μg, and more preferably at least about 50 μg, and more preferably at least about 75 μg, and more preferably at least about 100 μg, and more preferably at least about 250 μg, and more preferably at least about 500 μg, and more preferably at least about 750 μg, and more preferably at least about 1 mg, and more preferably at least about 5 mg, of an enzyme, an (isolated) protein comprising any of the proteins or homologues, variants, or fragments thereof discussed herein. Such a multi-enzyme composition may include any carrier with which the protein is associated by virtue of the protein preparation method, a protein purification method, or a preparation of the protein for use in any method according to the present invention. For example, such a carrier can include any suitable buffer, extract, or medium that is suitable for combining with the protein of the present invention so that the protein can be used in any method described herein according to the present invention.

In one embodiment of the invention, one or more enzymes in the multi-enzyme composition of the invention are bound to a solid support, i.e., an immobilized enzyme. As used herein, an immobilized enzyme includes immobilized isolated enzymes, immobilized microbial cells which contain one or more enzymes of the invention, other stabilized intact cells that produce one or more enzymes of the invention, and stabilized cell/membrane homogenates. Stabilized intact cells and stabilized cell/membrane homogenates include cells and homogenates from naturally occurring microorganisms expressing the enzymes of the invention and preferably, from genetically modified microorganisms as disclosed elsewhere herein. Thus, although methods for immobilizing enzymes are discussed below, it will be appreciated that such methods are equally applicable to immobilizing microbial cells and in such an embodiment, the cells can be lysed, if desired. A variety of methods for immobilizing an enzyme are disclosed in Industrial Enzymology 2nd Ed., Godfrey, T. and West, S. Eds., Stockton Press, New York, N.Y., 1996, pp. 267-272; Immobilized Enzymes, Chibata, I. Ed., Halsted Press, New York, N.Y., 1978; Enzymes and Immobilized Cells in Biotechnology, Laskin, A. Ed., Benjamin/Cummings Publishing Co., Inc., Menlo Park, Calif., 1985; and Applied Biochemistry and Bioengineering, Vol. 4, Chibata, I. and Wingard, Jr., L. Eds, Academic Press, New York, N.Y., 1983.

The PPO enzyme of the invention in the formulation may be an enzyme or protein as defined herein, e.g. a crude fermentation product, or a protein preparation that has been purified or partially purified (e.g., from a microorganism) using protein purification procedures known in the art, or an enzyme, protein or peptide that can be produced synthetically (e.g., chemically, such as by peptide synthesis) or recombinantly. The PPO in the formulation may also be a nucleic acid molecule as defined herein, e.g. a recombinant nucleic acid molecule and/or vector and/or host cell encoding said PPO enzyme as defined herein. The PPO comprising formulation may be a cell-free composition or a composition comprising a host cell which comprises a nucleic acid molecule encoding the PPO as defined herein, which is preferably capable of expressing the PPO as defined herein. The same description applies to reference to other enzymes, proteins or peptides described herein and to other microbial sources for such proteins or peptides.

The enzyme or multi-enzyme compositions, in some embodiments, comprise microorganisms or a crude fermentation product of microorganisms. A crude fermentation product refers to the fermentation broth, which has been separated from the microorganism biomass (by filtration, for example). In general, the microorganisms are grown in fermenters, optionally centrifuged or filtered to remove biomass, and optionally concentrated, formulated, and dried to produce an enzyme(s) or a multi-enzyme composition that is a crude fermentation product. In other embodiments, enzyme(s) or multi-enzyme compositions produced by the microorganism (including a genetically modified microorganism as described below) are subjected to one or more purification steps, such as ammonium sulfate precipitation, chromatography, and/or ultrafiltration, which result in a partially purified or purified enzyme(s). If the microorganism has been genetically modified to express the enzyme(s), the enzyme(s) will include recombinant enzymes. If the genetically modified microorganism also naturally expresses the enzyme(s) or other enzymes useful for lignocellulosic saccharification or any other useful application mentioned herein, the enzyme(s) may include both naturally occurring and recombinant enzymes.

Preferably, the enzyme or multi-enzyme composition for use in a method of the invention comprising at least about 500 μg, and preferably at least about 1 μg, and more preferably at least about 5 μg, and more preferably at least about 10 μg, and more preferably at least about 25 μg, and more preferably at least about 50 μg, and more preferably at least about 75 μg, and more preferably at least about 100 μg, and more preferably at least about 250 μg, and more preferably at least about 500 μg, and more preferably at least about 750 μg, and more preferably at least about 1 mg, and more preferably at least about 5 mg, of an enzyme, an (isolated) protein comprising any of the proteins or homologues, variants, or fragments thereof discussed herein. Such a multi-enzyme composition may include any carrier with which the protein is associated by virtue of the protein preparation method, a protein purification method, or a preparation of the protein for use in any method according to the present invention. For example, such a carrier can include any suitable buffer, extract, or medium that is suitable for combining with the protein of the present invention so that the protein can be used in any method described herein according to the present invention.

In one embodiment of the invention, one or more enzymes for use in a method of the invention are bound to a solid support, i.e., an immobilized enzyme. As used herein, an immobilized enzyme includes immobilized isolated enzymes, immobilized microbial cells which contain one or more enzymes of the invention, other stabilized intact cells that produce one or more enzymes of the invention, and stabilized cell/membrane homogenates. Stabilized intact cells and stabilized cell/membrane homogenates include cells and homogenates from naturally occurring microorganisms expressing the enzymes of the invention and preferably, from genetically modified microorganisms as disclosed elsewhere herein. Thus, although methods for immobilizing enzymes are discussed below, it will be appreciated that such methods are equally applicable to immobilizing microbial cells and in such an embodiment, the cells can be lysed, if desired. A variety of methods for immobilizing an enzyme are disclosed in Industrial Enzymology 2nd Ed., Godfrey, T. and West, S. Eds., Stockton Press, New York, N.Y., 1996, pp. 267-272; Immobilized Enzymes, Chibata, I. Ed., Halsted Press, New York, N.Y., 1978; Enzymes and Immobilized Cells in Biotechnology, Laskin, A. Ed., Benjamin/Cummings Publishing Co., Inc., Menlo Park, Calif., 1985; and Applied Biochemistry and Bioengineering, Vol. 4, Chibata, I. and Wingard, Jr., L. Eds, Academic Press, New York, N.Y., 1983.

In a fifth aspect, the invention provides for a method/process to produce an enzyme of the first aspect and/or multi-enzyme composition of the fourth aspect. Preferably, said method or process encompasses the step of transfecting a host cell with one or more nucleic acid molecules, for example expression vectors, which may be expression vectors of the second aspect, for example in a host cell which may be as defined in the third aspect. One or more protein(s) encoded by said nucleic acid molecule or expression vector may be produced by culturing the transfected host cell under conditions effective to produce the protein. Encompassed herein is a host cell produces at least two enzymes of a multi-enzyme complex as defined in the fourth aspect herein, preferably said at least two enzymes or the PPO of the invention and the AA9 LPMO as defined herein. In some instances, the protein may be recovered, and in others, the cell may be harvested in whole, either of which can be used in a composition. Microorganisms for use as host cells are cultured in an appropriate fermentation medium. An appropriate, or effective, fermentation medium refers to any medium in which the hosed cell, when cultured, is capable of expressing the enzyme of the invention. The microorganisms can be cultured by any fermentation process which includes, but is not limited to, batch, fedbatch, cell recycle, and continuous fermentation. In general, fungal strains are grown in fermenters, optionally centrifuged or filtered to remove biomass, and optionally concentrated, formulated, and dried to produce an enzyme(s) or a multi-enzyme composition that is a crude fermentation product. Particularly suitable conditions for culturing filamentous fungi are described, for example, in U.S. Pat. No. 6,015,707 and U.S. Pat. No. 6,573,086, supra.

Optionally, the method or process further comprises recovering the enzyme and/or multi-enzyme composition of the invention. Depending on the vector and host system used for production, resultant proteins may either remain within the recombinant cell; be secreted into the culture medium; be secreted into a space between two cellular membranes; or be retained on the outer surface of a cell membrane. The phrase “recovering the enzyme” refers to collecting the whole culture medium containing the protein and optionally implies additional steps of separation or purification. The enzyme and/or multi-enzyme composition produced can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential precipitation or solubilisation, resulting in substantially pure enzyme. As used herein, “substantially pure” refers to a purity that allows for the effective use of the enzyme in any method according to the present invention, i.e. substantially free of contaminants, other proteins and/or chemicals that might interfere or that would interfere with its use in a method of the present invention (e.g., that might interfere with enzyme activity), or that at least would be undesirable for inclusion with an enzyme of the invention when it is used in a method of the present invention (described in detail below). Preferably, a “substantially pure” enzyme, as referenced herein, is an enzyme that can be produced by any method (i.e., by direct purification from a natural source, recombinantly, or synthetically), and that has been purified from other protein components such that the enzyme makes up at least about 80% weight/weight of the total protein in a given composition (e.g., the enzyme of interest is about 80% of the protein in a solution/composition/buffer), and more preferably, at least about 85%, and more preferably at least about 90%, and more preferably at least about 91%, and more preferably at least about 92%, and more preferably at least about 93%, and more preferably at least about 94%, and more preferably at least about 95%, and more preferably at least about 96%, and more preferably at least about 97%, and more preferably at least about 98%, and more preferably at least about 99%, weight/weight of the total protein in a given composition.

In a sixth aspect, the invention provides for a method of demethylation (or demethylation method) of an R-substituted mono- or di-methoxyphenol in a R-substituted mono- or di-methoxyphenol comprising substrate, wherein said method comprises the step of contacting said substrate with an enzyme of the first aspect and/or a multi-enzyme composition of the fourth aspect. Preferably, the R-substituted mono- or di-methoxyphenol comprising substrate is lignin or lignin biomass or lignin biomiass saccharification mixture or a lignin derived compound, more preferably lignin or lignin biomass or lignin biomass saccharification mixture rich in S-groups and/or G-groups, most preferably rich in S-groups. Lignin or lignin biomass rich in S-groups and/or G groups is understood herein as being lignin or lignin biomass wherein the total percentage of S and G units together are at least 80% of the total amount of S, G and H. Examples of such biomass resources being rich in S and G units are: wheat straw, rice straw, rey straw, hemp, tall fescue, jute, sisal, banana plant leaf, loblolly pine, spurce (Picea Abies), Eucalyptus globus, Eucalyptus grandis, Birch pendula, Beech, Acacia, Carpinus betulus MWL (Milled-Wood Lignin), Eucryphia codrifolia MWL, Bambusa sp. MWL and Fagurs sylfatica. Examples thereof that are specifically rich in S units (i.e. at least 60% of the total of S, G and H): jute, sisal, Eucalyptus globus, Eucalyptus grandis, Birch pendula, Carpinus betulus MWL, Fagus sylvatica. Preferably, said lignin is low molecular lignin suchs as Biolignin and/or organosolv lignin. The R-substituted mono- or di-methoxyphenol comprising substrate may also be any R-substituted mono- or di-methoxyphenol comprising substrate that is not lignin-derived. Preferred R-substituted mono- or di-methoxyphenols comprised within said substrate are selected from, but not limited to, the group consisting of: syringic acid, vanillic acid, 3,4-dihydroxy-5-methoxybenzoic acid, sinapic acid, ferulic acid, 3,4-dihydroxy-5-methoxycinnamic acid, syringol, 2-methoxyphenol (guaiacol), 3-methoxycatechol and any combination thereof.

Preferably, R-substituted mono- or di-methoxyphenol comprising substrate further comprises between about 1 and 1000 μM of a copper salt, preferably between 20 and about 1000 μM a copper salt, wherein said copper salt is preferably selected from the group consisting of CuSO4, CuCl₂, CuBr₂, CuF₂, Cu(OH)₂ and Cu(NO₃)₂. Preferably the pH between of the R-substituted mono- or di-methoxyphenol comprising substrate is between about 5.0 and about 8.0. Preferably, the method step is performed at a temperature between about 20 and about 60° C. Even more preferably, the R-substituted mono- or di-methoxyphenol comprising substrate further comprises between about 1 and about 1000 μM a copper salt, preferably between 20 and about 1000 μM a copper salt, wherein said copper salt is preferably selected from the group consisting of CuSO4, CuCl₂, CuBr₂, CuF₂, Cu(OH)₂ and Cu(NO₃)₂, has a pH between about 5.0 and about 8.0 and the method step is performed at a temperature between about 20 and about 60° C.

Preferably, the demethylation method of the invention is part of a larger process (i.e. comprising additional steps) as detailed below. Preferably, said process further comprises the step of contacting the substrate with any further enzyme of the specific multi-enzyme composition as indicated to be useful for said process as detailed in the fourth aspect herein, either at once (for instance via the addition of a multi-enzyme composition) or sequentially.

Prerably, the demethylation method of the invention is part of a process to convert lignin into a value-added product, e.g. the production of biofuel, macromolecules and/or aromatic chemicals.

In an embodiment, the demethylation method of the invention is part of the process to produce of macromolecules such as dispersants, emulsifiers, binders, sequestrants, carbon fibres, polymer modifiers, resins and adhesives, wherein the production process takes advantage of lignin's polymer and polyelectrolyte properties. Such process preferably further comprises the step of sulfonation, introduction of ethyoxy, epoxy, vinyl and/or carbonyl moieties, depolymerization, intermolecular cross-linking, increasing phenolic functionality (such as carbonylation, carboxylation, amination, epoxidation and de-etherification) and/or melt-spinning.

In a further embodiment, the demethylation method of the invention is part of the process to produce aromatic monomers such as benzene, toluene, xylene, phenol, terephthalic acid monomeric lignin molecules. Preferably, such process further comprises the step of catalysis, such as dehyroxylation, dealkylation, hydrogenolysis, depolymerization, polymerization, sulfonation. A depolymerization step is preferably a selective depolymerziation step. A selective depolymerization step may give rise to complex aromatics that are difficult to make via conventional petrochemical routes. In turn, these aromatic monomers may serve as building blocks for further conversion such as polymerization.

In a further embodiment, the demethylation method of the invention may be part of a process to obtain fermentable products, such as sugars (saccarification process), from degrading biomass and/or lignocellulosic material, preferably biomass rich in (hemi-)cellulose. Preferably, said lignocellulosic material is agro-waste or a residue produced by agriculture and forestry. The lignocellulosic material may be partially or completely degraded to fermentable sugars. Economical levels of degradation at commercially viable costs are contemplated. Preferably, said method further comprises the step of contacting the substrate with at least one cellobiohydrolase, at least one xylanase, at least one endoglucanase, at least one β-glucosidase, at least one β-xylosidase, and at least one accessory enzyme, wherein these enzymes are composed in a multi enzyme composition as defined in the fourth aspect herein and/or are provided as individual compounds. Preferred endoglucanases, β-glucosidases, β-xylosidases, accessory enzymes and/or optional further enzymes useful in a saccarification process are defined in the fourth aspect herein.

Preferably, the demethylation method of the invention part of a process to dissolve pulp for the production of chemicals such as, but not limited to, rayon, cellophane and several chemicals such as cellulose esters (acetates, nitrates, propionates and butyrates) and cellulose ethers (carboxymethyl cellulose and methyl and ethyl cellulose) and ethanol (for instance as bioethanol bio fuel). Also preferred is the method of the invention for paper and pulp bleaching. The substrate comprising R-substituted mono- or di-methoxyphenol (either present already in the substrate or added as part of the method) in this embodiment comprises or consists of paper or pulp, preferably soft and/or hardwood pulp. The method of the invention may be used to improve bleachability of pulp in the pulp and paper industry.

In a further embodiment, the demethylation method of the invention is part of a process to degrade Distillers Dried Grain (DDG), wherein the substrate comprises preferably DDG derived from corn, to sugars. In certain embodiments, at least 10% of fermentable sugars are liberated. Preferably, at least 15% of the sugars are liberated, or at least 20% of the sugars are liberated, or at least 23% of the sugars are liberated, or at least 24% of the sugars are liberated, or at least 25% of the sugars are liberated, or at least 26% of the sugars are liberated, or at least 27% of the sugars are liberated, or at least 28% of the sugars are liberated.

Preferably, the demethylation process comprises a pretreatment process. In general, a pretreatment process will result in components of the lignocellulose being more accessible for downstream applications such as digestion by enzymes. The pretreatment can be a chemical, physical or biological pretreatment. The lignocellulose may have been previously treated to release some or all of the sugars, as in the case of DDG. Physical treatments, such as grinding, boiling, freezing, milling, vacuum infiltration, and the like may also be used with the methods of the invention. In one embodiment, the heat treatment comprises heating the lignocellulosic material to 121° C. for 15 minutes. A physical treatment such as milling can allow a higher concentration of lignocellulose to be used in the methods of the invention. A higher concentration refers to about 20%, up to about 25%, up to about 30%, up to about 35%, up to about 40%, up to about 45%, or up to about 50% lignocellulose. The lignocellulose may also be contacted with a metal ion, ultraviolet light, ozone, and the like. Additional pretreatment processes are known to those skilled in the art, and can include, for example, organosolv treatment, steam explosion treatment, lime impregnation with steam explosion treatment, hydrogen peroxide treatment, hydrogen peroxide/ozone (peroxone) treatment, acid treatment, dilute acid treatment, and base treatment, including ammonia fiber explosion (AFEX) technology. Details on pretreatment technologies and processes can be found in Wyman et al., Bioresource Tech. 96:1959 (2005); Wyman et al., Bioresource Tech. 96:2026(2005); Hsu, “Pretreatment of biomass” In Handbook on Bioethanol: Production and Utilization, Wyman, Taylor and Francis Eds., p. 179-212 (1996); and Mosier et al., Bioresource Tech. 96:673 (2005).

In some embodiments, the method/process steps may be performed one or more times in whole or in part. That is, one may perform one or more pretreatments, followed by one or more reactions with a composition of a method of the invention. The enzymes may be added in a single dose, or may be added in a series of small doses. Further, the entire process may be repeated one or more times as necessary. Therefore, one or more additional treatments with heat and enzymes are contemplated.

The methods described above result in the production of fermentable sugars. During, or subsequent to the method of the invention, the fermentable sugars may be recovered and/or purified by any method known in the art. The sugars can be subjected to further processing; e.g., they can also be sterilized, for example, by filtration.

In an additional embodiment, the invention provides a method/process, comprising the demethylation method the invention, that is for producing an organic substance, comprising saccharifying a lignocellulo sic material with an effective amount of the enzyme of the invention and preferably at least an LPMO as defined herein for use of a method of the present invention, fermenting the saccharified lignocellulosic material obtained with one or more microorganisms, and recovering the organic substance from the fermentation. Sugars released from biomass can be converted to useful fermentation products including but not limited to amino acids, vitamins, pharmaceuticals, animal feed supplements, specialty chemicals, chemical feedstocks, plastics, solvents, fuels, or other organic polymers, lactic acid, and ethanol, including fuel ethanol. Specific products that may be produced by the methods of the invention include, but not limited to, biofuels (including ethanol); lactic acid; plastics; specialty chemicals; organic acids, including citric acid, succinic acid, itaconic and maleic acid; solvents; animal feed supplements; pharmaceuticals; vitamins; amino acids, such as lysine, methionine, tryptophan, threonine, and aspartic acid; industrial enzymes, such as proteases, cellulases, amylases, glucanases, lactases, lipases, lyases, oxidoreductases, and transferases; and chemical feedstocks. The methods of the invention are also useful to generate feedstocks for fermentation by fermenting microorganisms. In one embodiment, the method further comprises the addition of at least one fermenting organism. As used herein, “fermenting organism” refers to an organism capable of fermentation, such as bacteria and fungi, including yeast. Such feedstocks have additional nutritive value above the nutritive value provided by the liberated sugars.

The one or more additional enzymes for use in a method/process of the present invention preferably also comprise enzyme combinations that break down or modify lignin or lignin-containing material, reducing or preventing unwanted adsorption of other components of multi-enzyme compositions applied. Such enzyme combinations or mixtures can include a multi-enzyme composition that contains at least one protein of the host organism or one or more enzymes or other proteins from other microorganisms, plants, or similar organisms. Synergistic enzyme combinations and related methods are contemplated. The invention includes methods to identify the optimum ratios and compositions of enzymes with which to degrade each lignin and lignocellulosic material. These methods entail tests to identify the optimum enzyme composition and ratios for efficient conversion of any biomass substrate to its constituent sugars. The Examples below include assays that may be used to identify optimum ratios and compositions of enzymes with which to degrade lignocellulosic materials.

The demethylation method of the invention may also be part of a process that is for use is a method for clarification and/or increasing the rate of filtration and/or decreasing haze formation of juices and/or beverages such as wine and beer. In this embodiment, the R-substituted mono- or di-methoxyphenol-comprising substrate (either present or added as specified in the seventh aspect herein) preferably comprises or consists of juice from fruit or vegetable such as, but not limited to, apple-, pineapple-, orange- and tomotato-, prune-, cranberry-juice, and/or cereals, such as, but not limited to barley, corn, wheat, rye, oats and maize. In this embodiment, the one or more additional enzymes for use in a method of the invention further preferably comprise an LPMO (preferably the AA9 LPMO as defined herein above), pectinase, carboxymethylcellulase and/or amylase.

In a further embodiment, the method of the demethylation method of invention is part of a process for macerating vegetables and/or fruit. In this embodiment, the R-substituted mono- or di-methoxyphenol-comprising substrate (either present or added as specified in the seventh aspect herein) is a fruit and/or a vegetable. In this embodiment, the one or more additional enzymes for use in a method of the invention further preferably comprise an LPMO (preferably the AA9 LPMO as defined herein above), pectinase, carboxymethylcellulase and/or amylase.

In another embodiment, the demethylation method of the invention is part of a process for extraction of coffee, plant oils, and starch, wherein the method comprises at least contacting the substrate with the enzyme of the invention and an LPMO (preferably the AA9 LPMO as defined herein above).

In another embodiment, the demethylation method of the invention is part of a process the production of xylitol, a valuable sweetener that has applications in both the pharmaceutical and food industries, wherein the method comprises at least contacting the substrate with the enzyme of the invention and an LPMO (preferably the AA9 LPMO as defined herein above).

In another embodiment, the demethylation method of the invention is part of a process to increase digestibility and/or nutritional properties of animal feedstocks or animal food, such as, but not limited to, agricultural silage and grain feed, wherein the method comprises at least contacting the substrate with the enzyme of the invention and an LPMO (preferably the AA9 LPMO as defined herein above). Said animal feedstock preferably contains cereals (e.g. barley, wheat, maize, rye or oats) or cereal by-products. Preferably, the substrate in this embodiment comprises or consists of animal feedstock such as, but not limited to, agricultural silage and grain feed, preferably comprising cereals, cereal and/or cereal solution.

In another embodiment, the demethylation method of the invention is part of a process to increase the quality of bakery products and/or improve textual characteristics (break and shred quality and crumb quality) of the bakery products such as, but not limited to bread. Preferably, the method of the invention is applied in order to increase the volume of the bakery product, e.g. a high-rising and/or light loaf of whole meal bread. In this embodiment, the one or more additional enzymes for use in a method of the invention further preferably comprise an LPMO (preferably the AA9 LPMO as defined herein above), a cellulase, such as beta-glucanase, cellulase, cellobiohydrolase and/or beta-glucosidase. Preferably, the cellulase preparation is a complex of cellulases and/or hemicellulases, preferably obtained from Trichoderma sp., more preferably from T. reesei. Also preferred are cellulase preparations obtained from Aspergillus, preferably obtained from A. niger. A suitable cellulase composition may be CELLUCLAST™ (available from Novozymes) or Bakezyme® XU, Bakezyme® XE, BakeZyme® X-cell or Bakezyme® W (all available from DSM). The one or more additional enzymes for use in a method of the invention may further comprise additional enzymes, e.g., glucoamylase, α-amylase, xylanase, protease, lipase, phospholipase may be used together with the LPMO in the dough or the composition.

The additional enzyme may be of any origin, including mammalian and plant, and preferably of microbial (bacterial, yeast or fungal) origin. The glucoamylase for use in the present embodiment also include glucoamylases having a sequence identity of at least 50%, at least 60%, at least 65%, at least 70%, at least 75%, at least 80%, at least 85%, at least 90%, at least 95%, at least 96%, at least 97%, at least 98%, or at least 99% to the amino acid sequence of the A. niger G1 or G2 glucoamylase (Boel et al. (1984), EMBO J. 3(5), p. 1097-1102), the A. awamori glucoamylase disclosed in WO84/02921, or the A. oryzae glucoamylase (Agric. Biol. Chem. (1991), 55 (4), p. 941-949). The amylase may be fungal or bacterial, e.g. a maltogenic α-amylase from B. stearothermophilus or an α-amylase from Bacillus, e.g. B. licheniformis or B. amyloliquefaciens, a beta-amylase, e.g. from plant (e.g. soy bean) or from microbial sources (e.g. Bacillus), a glucoamylase, e.g. from A. niger, or a fungal α-amylase, e.g. from A. oryzae. Suitable commercial maltogenic α-amylases include NOVAMYLO (Novozymes A/S) and OPTICAKE® (Novozymes A/S). Suitable commercial fungal α-amylase compositions include, e.g., BAKEZYME P 300 (available from DSM) and FUNGAMYL 2500 SG, FUNGAMYL 4000 BG, FUNGAMYL 800 L, FUNGAMYL ULTRA BG and FUNGAMYL ULTRA SG (available from Novozymes A/S). The hemicellulase may be a pentosanase, e.g. a xylanase which may be of microbial origin, e.g. derived from a bacterium or fungus, such as a strain of Aspergillus, in particular of A. aculeatus, A. niger, A. awamori, or A. tubigensis, from a strain of Trichoderma, e.g. T. reesei, or from a strain of Humicola, e.g. H. insolens. Suitable commercially available xylanase preparations for use in the present invention include PENTOPAN MONO BG and PENTOPAN 500 BG (available from Novozymes), GRINDAMYL POWERBAKE (available from Danisco), and BAKEZYME BXP 5000 and BAKEZYME BXP 5001 (available from DSM). The protease may be from Bacillus, e.g. B. amyloliquefaciens. The lipase may be derived from a strain of Thermomyces (Humicola), Rhizomucor, Candida, Aspergillus, Rhizopus, or Pseudomonas, in particular from T. lanuginosus (H. lanuginosa), Rhizomucor miehei, C. antarctica, A. niger, R. delemar, R. arrhizus or P. cepacia. The phospholipase may have phospholipase A1, A2, B, C, D or lysophospholipase activity; it may or may not have lipase activity. It may be of animal origin, e.g. from pancreas, snake venom or bee venom, or it may be of microbial origin, e.g. from filamentous fungi, yeast or bacteria, such as Aspergillus or Fusarium, e.g. A. niger, A. oryzae or F. oxysporum. A preferred lipase/phospholipase from F. oxysporum is disclosed in WO 98/26057. Also, the variants described in WO 00/32758 may be used. The additional enzyme may be of any origin, including mammalian and plant, and preferably of microbial (bacterial, yeast or fungal) origin and may be obtained by techniques conventionally used in the art. Suitable phospholipase compositions are LIPOPAN F and LIPOPAN XTRA (available from Novozymes) or PANAMORE GOLDEN and PANAMORE SPRING (available from DSM). In this embodiment, the R-substituted mono- or di-methoxyphenol-comprising substrate (either present or added as specified in the seventh aspect herein) preferably comprises or consists of a baking composition or a dough, preferably comprising a flour such as whole meal flour. Whole meal flour is a flour which may be derived by grinding or mashing the whole cereal grain. When used in baking it is typically added to other more refined white flours to provide nutrients (especially fiber and protein), texture, and body to the finished product. The word “whole” refers to the fact that not only the starchy endosperm but also the bran and germ are used in making the flour.

In another embodiment, the demethylation method of the invention is part of a process to produce xylo-oligomers (XOs), or oxidised xylo-oligomers, which may be used in pharmaceutical, agriculture and feed products. XOs are known to have pre-biotic effects, as they are neither hydrolyzed nor absorbed in the upper gastrointestinal tract, and they affect the host by selectively stimulating the growth or activity of one or a number of bacteria in the colon, thus improving health. Furthermore, XOs are believed to reduce cholesterol levels, maintenance of gastrointestinal health, and improvement of the biological availability of calcium. Furthermore, XOs inhibit starch retrogradation, improving the nutritional and sensory properties of food.

The demethylation method of the invention may be part of a process wherein ferulic acid is present. The demethylation method may be part of a process wherein a substrate is contacted with ferulic acid esterase (FAE) under conditions whereby R-substituted mono-methoxyphenol is produced. The substrate may be a pretreated lignocellulosic biomass substrate. The substrate may be contacted with ferulic acid esterase, LPMO, and PPO. The ferulic acid esterase may be at least about 70% identical, at least about 80% identical, at least about 90% identical, at least about 95% identical, or at least about 98% to the amino acid sequence of XynZ from Clostridium thermocellum (SEQ ID NO:73), Type D FAE from Neurospora crassa (SEQ ID NO: 74), Type C FAE from Talaromyces stipitatus (SEQ ID NO: 75), FAE from Thielavia terrestris (SEQ ID NO: 76), from Humicola insolens (SEQ ID NO: 77), from Ruminococcus sp. (SEQ ID NO: 78), from PC-2 from Orpinomyces sp. (SEQ ID NO: 79), from Schizophyllum commune (SEQ ID NO: 80, SEQ ID NO: 81), from Aspergillus oryzae (SEQ ID NO: 82), type A FAE from Aspergillus niger (SEQ ID NO: 83), type B FAE from Aspergillus niger (SEQ ID NO: 84), type C FAE from Emericella nidulans (SEQ ID NO: 85), from Fusarium oxysporum (SEQ ID NO: 86), FaeA1 from Myceliophthora thermophila C1 (SEQ ID NO: 87), FaeA2 from Myceliophthora thermophila C1 (SEQ ID NO: 88), FaeB1 from Myceliophthora thermophila C1 (SEQ ID NO: 89), or FaeB2 from Myceliophthora thermophila C1 (SEQ ID NO: 90). Where the sequence given comprises a signal sequence, one of skill in the art will appreciate that the ferulic acid esterase may be at least about 70% identical, at least about 80% identical, at least about 90% identical, at least about 95% identical, or at least about 98% to the amino acid sequence of the mature sequence (which does not comprise the signal sequence). The LPMO may be at least about 70% identical, at least about 80% identical, at least about 90% identical, at least about 95% identical, or at least about 98% to the amino acid sequence of SEQ ID NO: 1 or to SEQ ID NO: 93. The PPO may be at least about 70% identical, at least about 80% identical, at least about 90% identical, at least about 95% identical, or at least about 98% identical to the amino acid sequence of SEQ ID NO: 37, 41, or 45. The LPMO may have the amino acid sequence of SEQ ID NO: 1 or SEQ ID NO: 93. The PPO may have the amino acid sequence of SEQ ID NO: 41. The PPO may have the amino acid sequence of SEQ ID NO: 45.

Typically, the amount of PPO in a composition for use in a method/process of the invention will depend on the amount of R-substituted mono- or di-methoxyphenols present in the substrate to be degraded and/or modified. The amount of enzyme (herein to be understood as PPO and/or further enzyme) may be from about 0.1 to about 200 mg enzyme or enzyme composition per gram of R-substituted mono- or di-methoxyphenols; or from about 3 to about 20 mg enzyme or enzyme composition per gram of R-substituted mono- or di-methoxyphenols. The invention encompasses the use of any suitable or sufficient amount of PPO between about 0.1 mg and about 200 mg PPO per gram R-substituted mono- or di-methoxyphenols, in increments of 0.05 mg (i.e., 0.1 mg, 0.15 mg, 0.2 mg . . . 199.9 mg, 199.95 mg, 200 mg).

Furthermore, the amount of PPO in a composition for use in a method/process of the invention may be between about 0.5 to about 1000 mg enzyme per kg dry matter of the R-substituted mono- or di-methoxyphenol comprising substrate, or 0.5-100 mg/kg, or 0.5-100 mg/kg or 0.5-50 mg/kg, or 1-25 mg/kg or 1-15 mg/kg, or 2-10 mg/kg enzyme/kg dry matter of the R-substituted mono- or di-methoxyphenol-comprising substrate.

The invention also relates to a commercial process comprising the demethylation method of the invention, such as washing or treating of clothing or fabrics, detergent processes, animal feed, food, baking, beverage, biofuel, starch preparation, liquefaction, biorefining, de-inking and biobleaching of paper and pulp, oil and waste dispersing, and treatment of waste streams.

The PPO for use in a method of the invention may be an enzyme or protein as defined herein. The PPO for use in a method of the invention may also be a nucleic acid molecule, preferably a recombinant nucleic acid molecule and/or vector, encoding said PPO as defined herein. In an embodiment, the PPO for use in a method of the present invention is a part of a cell-free composition. In another embodiment, wherein the PPO for use in a method of the invention is a nucleic acid molecule, said nucleic acid molecule is part of a host cell as defined herein, preferably a genetically modified organism as defined herein, which is preferably modified to recombinantly express the PPO for use in a method of the invention.

In an embodiment, the invention provides the demethylation method of the invention wherein a genetically modified microorganism is cultivated in a nutrient medium comprising the R-substituted mono- or di-methoxyphenol to be demethylated.

When further enzymes are added following the demethylation method of the present invention, the conditions (such as temperature and pH) may be altered to those optimal for the further enzyme before, during, or after addition of the further enzyme. Multiple rounds of enzyme addition are also encompassed. The further enzyme may also be present in the R-substituted mono- or di-methoxyphenol-comprising substrate itself as a result of genetically modifying the plant. The nutrient medium used in a fermentation reaction can also comprise one or more accessory enzymes.

In a seventh aspect, the invention provides a method or process for degrading and/or modifying (hemi-)cellulose, for example in a (hemi-)cellulose-comprising substrate, wherein said method comprises the step of contacting the (hemi-)cellulose-comprising substrate composition with an accessory enzyme as defined herein, preferably an LPMO (preferably the AA9 or LPMO9B LPMO as defined herein above), and a PPO. Preferably said PPO is a PPO of the invention as defined in aspect 1. The method may be a method for degrading or modifying xylan, for example in a xylan-comprising substrate.

An alternative PPO that can be used in the methods disclosed herein, other than the PPO of the invention, preferably is a PPO that comprises or consists of a common central tyrosinase domain, preferably of the family with NCBI accession number pfam00264 (Volbeda and Hol, J. Mol. Biol. Volume 209(2): pages 249-279, 1989) and preferably comprises or consists of an amino acid sequence that is at least 50%, 60%, 70%, 80%, more preferably at least 90%, 91%, 92%, 93%, 94%, even more preferably at least 95%, 96%, 97%, 98%, 99% or at least 100% identical to any one of SEQ ID NO: 49 (PPO1), SEQ ID NO: 53 (PPO3), SEQ ID NO: 57 (PPO8), SEQ ID NO: 61 (PPO5), SEQ ID NO: 65 (PPO10) or SEQ ID NO: 69 (PPO9). SEQ ID NO: 49, 53, 57, 61, 65 and 69 represent the mature form of PPO1, PPO3, PPO8, PPO5, PPO10 and PPO9, respectively. SEQ ID NO: 50, 54, 58, 62, 66 and 70 represent PPO1, PPO3, PPO8, PPO5, PPO10 and PPO9 including the signal sequence, respectively. Preferably said PPO is obtainable from a fungus, preferably of the genus Myceliophthora. Being “obtainable from” is to be understood herein as that the enzyme originates from the indicated organism, i.e. is naturally produced by the indicated organism as a native enzyme. More preferably, the PPO originates from M. thermophila C1 fungus or derivatives thereof, such as a M. thermophila C1 fungus selected from Garg 27K, (Accession No. VKM F-3500D), UV13-6 (Accession No. VKM F-3632 D); NG7C-19 (Accession No. VKM F-3633 D); UV18-25 (Accession No. VKM F-3631 D); strain W1L (Accession No. CBS122189) or W1L#100L (Accession No. CBS122190), preferably a M. thermophila C1 (Accession No. VKM F-3500D).

Preferably, the alternative PPO is encoded by a nucleic acid sequence has at least 50%, 60%, 70%, 80%, or more preferably at least 90%, 91%, 92%, 93%, 94%, 95%, 96%, 97%, 98%, 99% or 100% identity with SEQ ID NO: 51 (cDNA encoding PPO1), SEQ ID NO: 52 (gDNA encoding PPO1), SEQ ID NO: 55 (cDNA encoding PPO3), SEQ ID NO: 56 (gDNA encoding PPO3), SEQ ID NO: 59 (cDNA encoding PPO8), SEQ ID NO: 60 (gDNA encoding PPO8), SEQ ID NO: 63 (cDNA encoding PPO5), SEQ ID NO: 64 (gDNA encoding PPO5), SEQ ID NO: 67 (cDNA encoding PPO10), SEQ ID NO: 68 (gDNA encoding PPO10), SEQ ID NO: 71 (cDNA encoding PPO9) or SEQ ID NO: 72 (gDNA encoding PPO9). Preferably, said encoding nucleic acid sequence has at least 50%, 60%, 70%, 80%, or more preferably at least 90%, 91%, 92%, 93%, 94%, 95%, 96%, 97%, 98%, 99% or 100% identity with SEQ ID NO: 52 (gDNA encoding PPO1), SEQ ID NO: 56 (gDNA encoding PPO3), SEQ ID NO: 60 (gDNA encoding PPO8), SEQ ID NO: 64 (gDNA encoding PPO5), SEQ ID NO: 68 (gDNA encoding PPO10), or SEQ ID NO: 72 (gDNA encoding PPO9).

Depending on the amount of R-substituted mono- or di-methoxyphenol present in a hemicellulose-comprising substrate, the method further comprises the step of adding and/or admixing R-substituted mono- or di-methoxyphenol, preferably a substantial amount of R-substituted mono- or di-methoxyphenol.

In an embodiment of the invention, the (hemi-)cellulose-comprising substrate is substantially free of R-substituted mono- or di-methoxyphenol. A method or process of this embodiment comprises the step of adding a substantial amount of R-substituted mono- or di-methoxyphenol.

In a further embodiment, the (hemi-)cellulose-comprising substrate does comprise R-substituted mono- or di-methoxyphenol, however, in a sub-optimal amount or concentration or in a form that is less accessible for demethylation by the PPO enzyme of the invention. A method or process of this embodiment also comprises the step of adding a substantial amount of R-substituted mono- or di-methoxyphenol.

In yet a further embodiment, the (hemi-)cellulose-comprising substrate does comprise R-substituted mono- or di-methoxyphenol in optimal amount or concentration and in a form that accessible for demethylation by the PPO enzyme of the invention. A method or process of this embodiment no further step of adding a R-substituted mono- or di-methoxyphenol is required.

A method of this aspect and of each embodiment herein encompasses a method wherein the LPMO, the PPO and/or R-substituted mono- or di-methoxyphenol are contacted, added and/or admixed with the (hemi-)cellulose-comprising substrate simultaneously, and sequentially.

In embodiments, the PPO is PPO7 and the R-substituted mono- or di-methoxyphenol is syringic acid, synapic acid, 3-hydroxy-4-methoxycinnamic acid, 4-hydroxy-3-methoxyphenylactone, coniferyl aldehyde, ferulic acid, guaiacol, hesperidin, homovanillic acid, vanillic acid, 3,4-dihydroxybenzoic acid, caffeic acid, 3,4-dihydroxy-5-methoxycinnamic acid, or combinations thereof.

A substrate being substantially free of R-substituted mono- or di-methoxyphenol is understood herein as to comprise no appreciable amounts of R-substituted mono- or di-methoxyphenol, i.e. less than about 0.1%, more preferably less than about 0.01%, more preferably less than about 0.01% or most preferably less than about 0.0001% of the total dry weight of the substrate.

Adding or admixing a substantial amount of R-substituted mono- or di-methoxyphenol is understood herein as adding or admixing an amount of R-substituted mono- or di-methoxyphenol that results in an in increase in (hemi-)cellulose degradation and/or modification, optionally in an increase in XO production.

In embodiments, said (hemi-)cellulose-comprising substrate comprises xylan that consists of large unsubstituted, linear xylan chains, preferably such as is present in oat spelt xylan and birchwood xylan. Preferably, said xylan-comprising substrate does not consist only of, or preferably does not comprise mainly of, or even more preferably does not comprise, xylan that has arabinosyl-substituents present on the β-1,4-xylan backbone such as the branched xylan present in wheat arabinoxylan. Preferably, the xylan to be degraded and/or modified in the xylan-comprising substrate is xylan that consists of large unsubstituted, linear xylan chains, preferably such as is present in oat spelt xylan and birchwood xylan. Preferably, the xylan to be degraded and/or modified in the xylan-comprising substrate is not xylan that has arabinosyl-substituents present on the β-1,4-xylan backbone such as the branched xylan present in wheat arabinoxylan.

Preferably said (hemi-)cellulose-comprising substrate comprises cellulose, more preferably hemicelluloses-associated cellulose and/or amorphous cellulose. Optionally, said (hemi-)cellulose-comprising substrate of this method is substantially free of R-substituted mono- or di-methoxyphenol compounds as defined herein. Optionally, R-substituted mono- or di-methoxyphenol compounds as defined herein are added as part of the method, preferably before contacting the (hemi-)cellulose-comprising substrate with either of the accessory enzyme and the PPO.

Preferably, the R-substituted mono- or di-methoxyphenol to be added can be any R-substituted mono- or di-methoxyphenol comprising compound as defined herein. Preferred within this method are low molecular weight R-substituted mono- or di-methoxyphenol such as present in biolignin and/or organosolv lignin. Even more preferred for use in method of the present aspect is the use of monomers of syringic acid, vanillic acid, 3,4-dihydroxy-5-methoxybenzoic acid, sinapic acid, ferulic acid, 3,4-dihydroxy-5-methoxycinnamic acid, syringol, 2-methoxyphenol (guaiacol), 3-methoxycatechol or any combination thereof.

Preferably, the substrate further comprises between about 1 and 1000 μM of a copper salt, preferably between 20 and about 1000 μM a copper salt, wherein said copper salt is preferably selected from the group consisting of CuSO4, CuCl₂, CuBr₂, CuF₂, Cu(OH)₂ and Cu(NO₃)₂. Preferably the pH of the substrate is between about 5.0 and about 8.0. Preferably, the method is performed at a temperature between about 20 and about 60° C. Even more preferably, the substrate further comprises between about 1 and about 1000 μM a copper salt, preferably between 20 and about 1000 μM a copper salt, wherein said copper salt is preferably selected from the group consisting of CuSO4, CuCl₂, CuBr₂, CuF₂, Cu(OH)₂ and Cu(NO₃)₂, has a pH between about 5.0 and about 8.0 and the method is performed at a temperature between about 20 and about 60° C.

Preferably, the method of this aspect is part of a method or process for saccharification, for dissolving pulp for the production of chemicals, for paper and pulp bleaching, for DDG degradation, for producing an organic substance, for clarification and/or increasing the rate of filtration and/or decreasing haze formation of juices and/or beverages such as wine and beer, for extraction of coffee, plant oils, and starch, for production of xylitol, for increasing digestibility and/or nutritional properties of animal feedstocks or animal food, and for increasing the quality of bakery products and/or improve textual characteristics (break and shred quality and crumb quality) of the bakery products such as, but not limited to bread, as further detailed in the sixth aspect, and may comprise further steps as further detailed in the sixth aspect herein, such as contacting the substrate with further enzymes that are indicated to be particular suitable for said process as detailed in the fourth and sixth aspect herein.

In an eighth aspect, the invention provides for an R-substituted mono- or di-methoxyphenol-comprising substrate and/or a substrate as defined in any one of the preceding aspects, further comprising the PPO enzyme of the first aspect, the nucleic acid molecule of the second aspect, the host cell of the third aspect, the enzyme composition or formulation of the fourth aspect. Preferably, said R-substituted mono- or di-methoxyphenol-comprising substrate and/or a xylan-comprising substrate is for or suitable for use in a method or process of the sixth or seventh aspect.

In a ninth aspect, the invention provides for the use of an enzyme of the first aspect, a nucleic acid molecule of the second aspect, a host cell of the third aspect and/or an enzyme composition of the fourth aspect in a process of demethylation, and/or any method or process as defined in the sixth and/or seventh aspect herein.

### Example 1: Cloning of Myceliophthora thermophila C1 PPO Polypeptide Coding Sequences Having Demethylating Activity

Genomic DNA encoding PPO2 (SEQ ID NO: 36), PPO4 (SEQ ID NO: 40), PPO6 (SEQ ID NO: 44) and PPO7 (SEQ ID NO: 48) were synthesized (GeneArt) and cloned in pChi vector as previously described (WO 2010/107303 A2). Transformation of W1L#100.1Δpyr5ΔA1p1 strain using the pyr5 marker as previously described.

### Example 2: Preparation of PPO Polypeptides Having Demethylating Activity

PPO4, PPO6 and PPO7 having demethylating activity and PPO2 were prepared as described previously. In short, fermentation of W1L#100.1Δpyr5ΔAlp1 strain overexpressing the PPO were performed as described in WO 2010/107303 A2 broth was harvested, filtrated to remove fungal biomass, concentrated and dialyzed against 20 mM KPi pH 7.0 plus 150 mM KCl using a 5 kDa PES membrane (Vivacell® 70, Sartorius). Samples were subsequently freeze-dried for storage.

### Example 3: Catechol Oxidase Activity Assay

PPO activity was determined by measuring the initial rate of 3-methoxycatechol oxidation at 400 nm (extinction coefficient used: ε 1200 M⁻¹cm⁻¹). One unit of activity is defined as the amount of PPO activity that oxidizes 1 μmol of 3-methoxycatechol to quinone per minute. Substrate was used at a concentration of 4 mM.

### Example 4: Characterization of PPO Polypeptides Having Demethylating Activity

The pH optima were determined by measuring the initial rate of 3-methoxycatechol oxidation with varying pHs, at room temperature and using a CuSO₄ concentration of 10 μM. The pH was varied in the range of pH3-9 using Carmody buffers. Enzyme loading was applied resulting in A400 absorbance values in the range of 0.1-1. Protein concentration was determined using the BCA method.

Optimal copper concentrations for the PPOs were determined at the optimal pH by varying the concentration of CuSO4 in the range of 1-1000 μM. In these experiments the pH was controlled using 50 mM Bis-Tris buffer pH 7.0.

Temperature optima were determined by measuring A400 absorbance after 1 h incubation at temperatures in the range of 20 to 70° C. In these experiments the pH was controlled using 50 mM Bis-Tris buffer pH 7.0. Copper concentrations of (20 or 50 μM) were used to prevent chemical oxidation as background activity at higher copper concentrations.

TABLE 1
Optima determined for PPO 4, 6 and 7

| | | | | [CuSO₄] | | T |
|---|---|---|---|---|---|---|
| | | pH | [CuSO₄]ₒₚₜ | range*** | Tₒₚₜ | range** |
| Enzyme | pHₒₚₜ | range** | (μM) | (μM) | (° C.) | (° C.) |
| PPO4 | 7.0 | 5.0-7.5 | 200 | 20-1000 | 55 | 30-60 |
| PPO6 | 7.0 | 6.0-8.5 | 200 | 50-1000 | 30 | 20-50 |
| PPO7 | 7.0 | 6.5-8.0 | 400 | 100-1000 | 40 | 20-55 |

*PPO activity was determined by measuring the rate of 3-methoxycatechol oxidation under optimal conditions. 3-methoxy-catechol oxidation was followed at 400 nm (extinction coefficient used: ε 1200 M−1cm−1).
**pH and T range: >50% of maximum activity
***Copper concentration range: >90% of maximum activity

### Example 5: Purification of PPO7 Having Demethylating Activity and Purification of PPO2

Freeze-dried material of PPO7 was dissolved in MilliQ water, 0.45 μm filtered, applied on a 50 mL Source™ 30Q column (XK16/20, GE Healthcare) equilibrated with 20 mM KPi pH 7.0 and eluted using a gradient of 0 to 1 M KCl (stepwise) in this buffer at a flow rate of 3.5 mL per minute. As a second purification step, a 50 mL Phenyl Sepharose™ 6 Fast Flow (high sub) column (XK16/20, GE Healthcare) equilibrated with 10 mM KPi pH 7.0 plus 1 M ammonium sulphate was used. A stepwise elution to buffer without ammonium sulphate was performed at a flow rate of 3.5 mL per minute. Pools were concentrated and dialyzed against 20 mM KPi pH 7.0 plus 150 mM KCl using a 5 kDa PES membrane (Vivacell® 70, Sartorius). The final purification step was performed on a 120 mL Superdex® 200 PG column (HiLoad 16/600) equilibrated with 20 mM KPi pH 7.0 plus 150 mM KCl at a flow rate of 1 mL per minute. Pure fractions were pooled and concentrated using a 5 kDa PES membrane (Vivacell® 70, Sartorius).

Freeze-dried material of PPO2 was dissolved in MilliQ water, 0.45 μm filtered, applied on a 50 mL Source™ 30Q column (XK16/20, GE Healthcare) equilibrated with 10 mM KPi pH 7.0 and eluted using a gradient of 0 to 1 M KCl (stepwise) in this buffer at a flow rate of 3.5 mL per minute. As a second purification step, a 20 mL Butyl Sepharose™ Fast Flow column (HiPrep 16/10, GE Healthcare) equilibrated with 10 mM KPi pH 7.0 plus 1.5 M ammonium sulphate was used. A stepwise elution to buffer without ammonium sulphate was performed at a flow rate of 4 mL per minute. Pools were concentrated using a 5 kDa PES membrane (Vivacell® 70, Sartorius). Finally, size exclusion chromatography was performed on a 120 mL Superdex® 200 PG column (HiLoad 16/600) equilibrated with 20 mM KPi pH 7.0 plus 150 mM KCl at a flow rate of 1 mL per minute. Pure fractions were pooled and concentrated using a 5 kDa PES membrane (Vivacell® 70, Sartorius).

### Example 6: Conversion of Syringic Acid by PPO Polypeptides Having Demethylating Activity

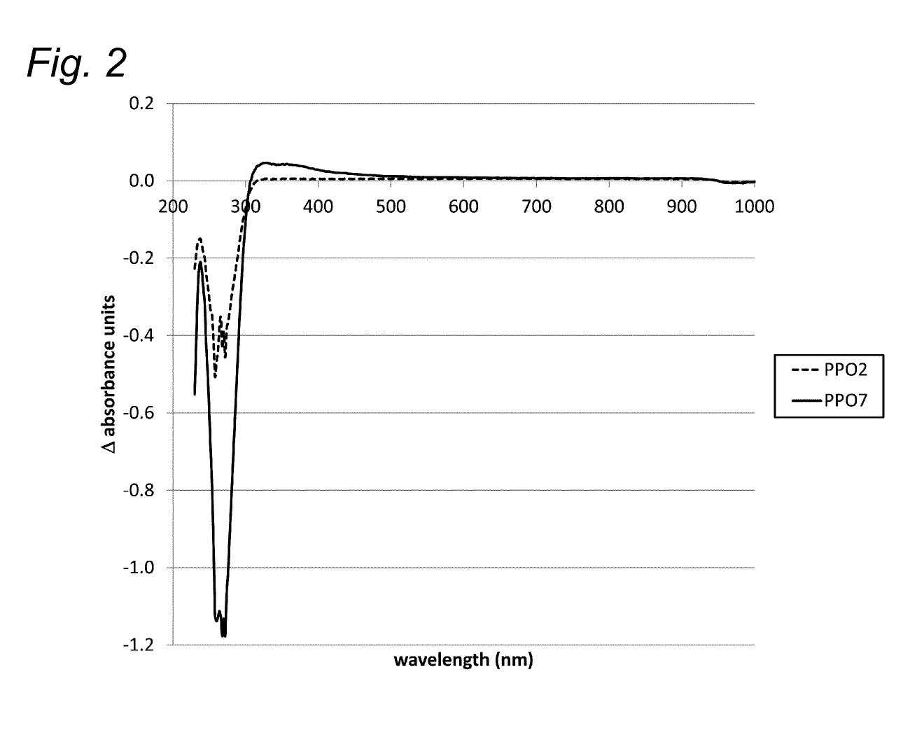

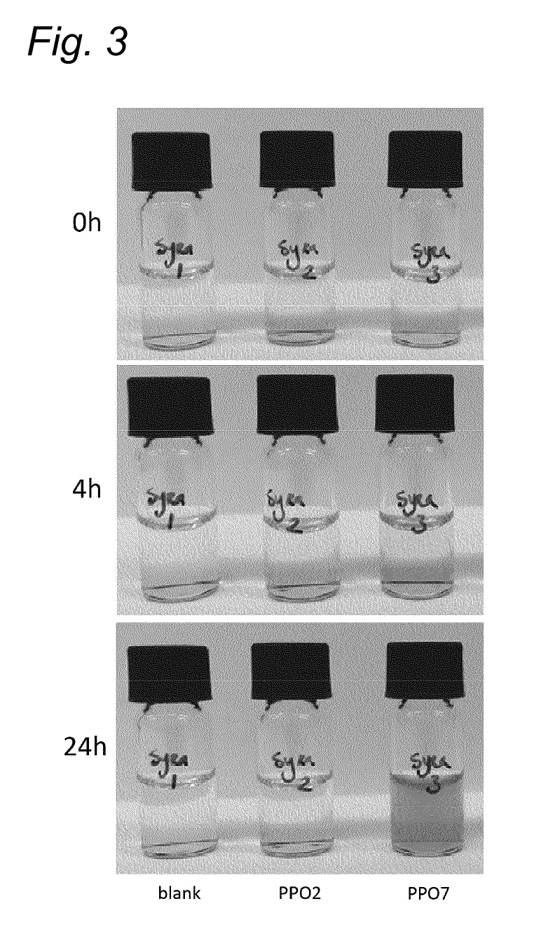

Syringic acid (12 mM, 2.38 g/L) was incubated with PPO7 and PPO2 from Example 5 (0.048 g/L, 2% loading (w/w)) in 50 mM BisTris buffer pH 6.5 with 10 μM CuSO₄. Blank prepared without the addition of enzyme was included. Incubations were performed in HPLC vials with a total of 750 μL reaction volume at room temperature for 24 h. The reaction was stopped by adding 21 μL 1M HCl to lower the pH to ˜3. Samples were filtrated and absorbance spectra were measured (230-1000 nm, 2 nm interval). FIG. 2 shows the difference absorbance spectra (syringic acid blank subtracted) after 24 h incubation. Syringic acid conversion induced the negative peak in the difference absorbance spectra between 230 and 300 nm, which was more significant for PPO7 than for PPO2. Moreover, PPO7 reaction products resulted in a spectral increase between 300 and 500 nm (which was confirmed by the visual colour change of the reaction vial; see FIG. 3). It is to be noted that any change in absorbance in time indicates syringic acid conversion.

### Example 7: Conversion of Syringic Acid, Sinapic Acid and Syringol by PPO Polypeptides Having Demethylating Activity

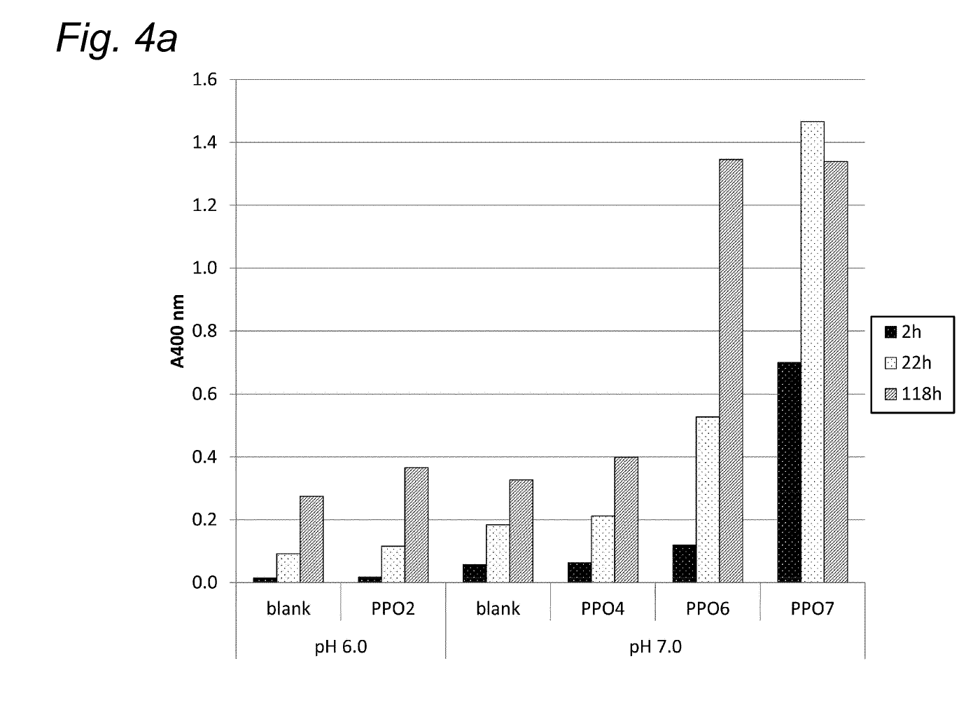

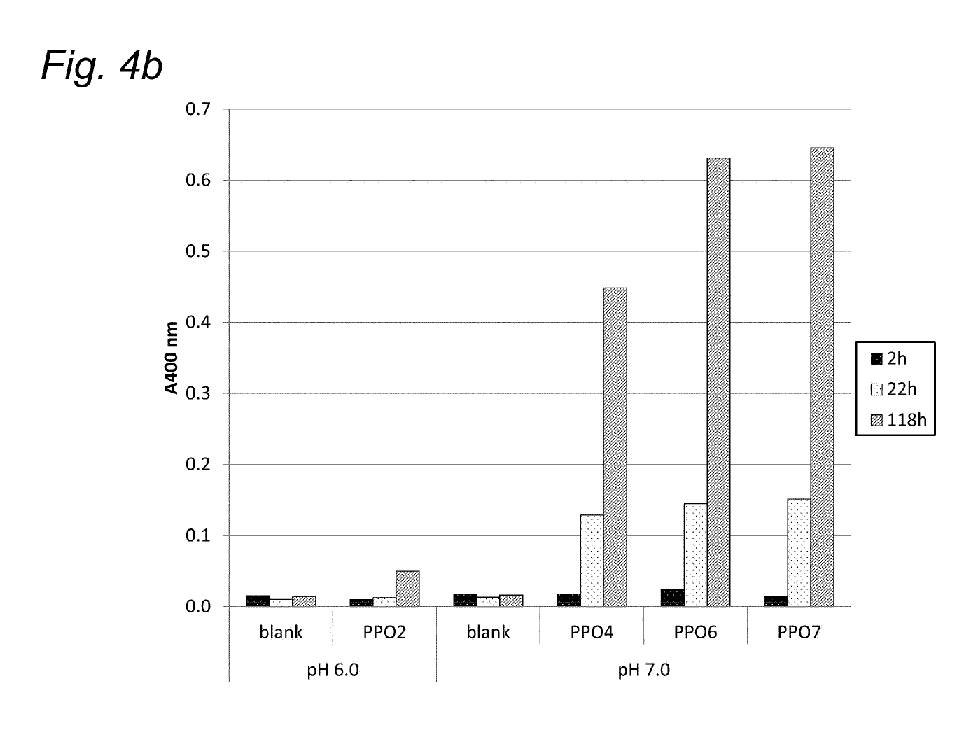

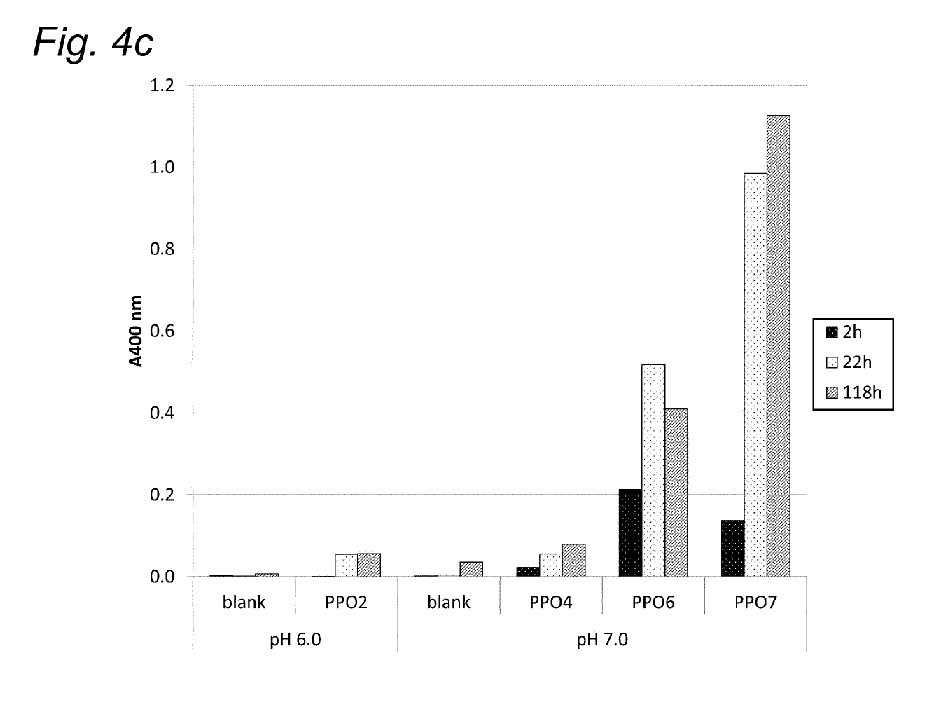

Syringic acid, sinapic acid and syringol (4 mM) were incubated with crude PPO2, 4, 6 and 7 from Example 5 in 100 mM BisTris buffer pH 6.0 or 7.0 with 50 μM CuSO4. Crude PPOs were dosed on target protein with a final concentration of 8.2 μg/mL. Substrate blanks prepared without the addition of enzyme and enzyme blanks without the addition of substrate were included. Incubations were performed in microtiterplates with a total reaction volume of 150 μL at room temperature for approximately 120 h. After 2, 22 and 118 h absorbance at 400 nm was measured. FIG. 4 shows UV-VIS data at 400 nm; enzyme blanks were subtracted.

PPO7 and PPO6 were able to convert syringic acid, sinapic acid and syringol, whereas PPO4 was only able to use syringic acid as a substrate. PPO2 showed no significant activity on those substrates compared to the substrate blanks.

### Example 8: Demethylation of Syringic Acid

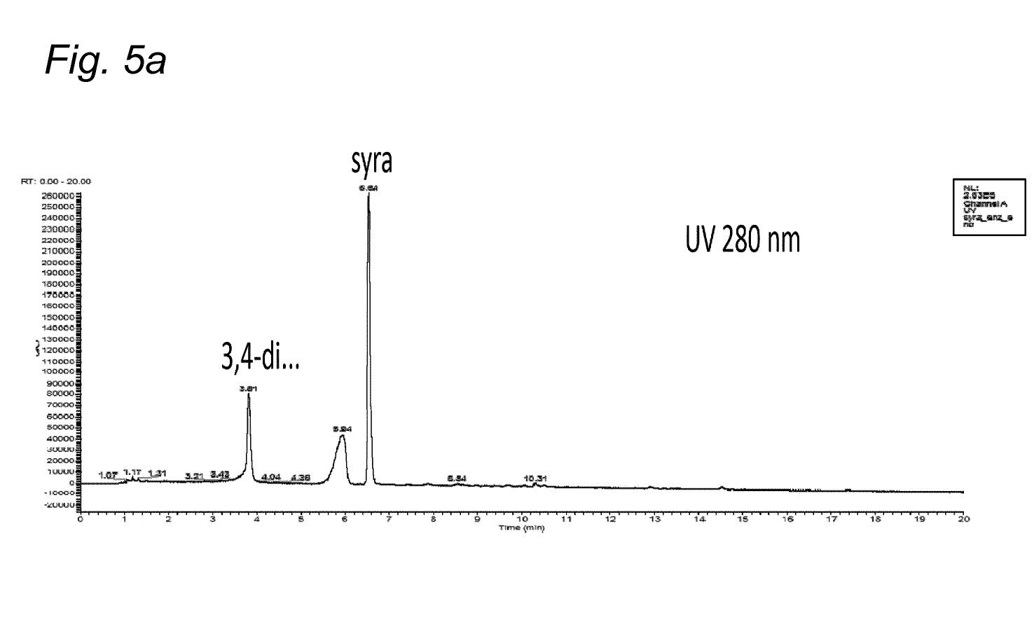

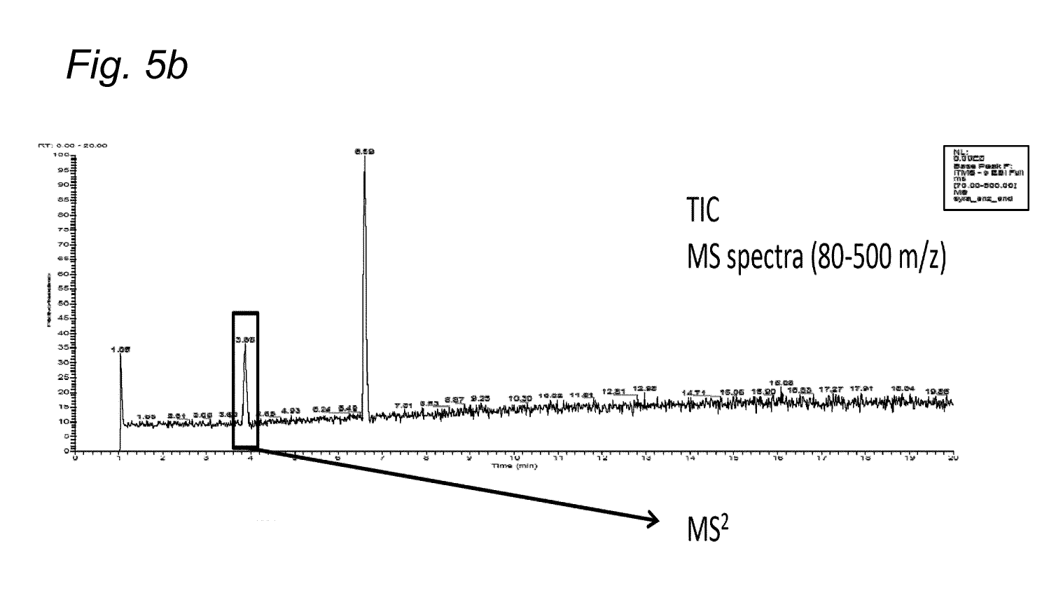

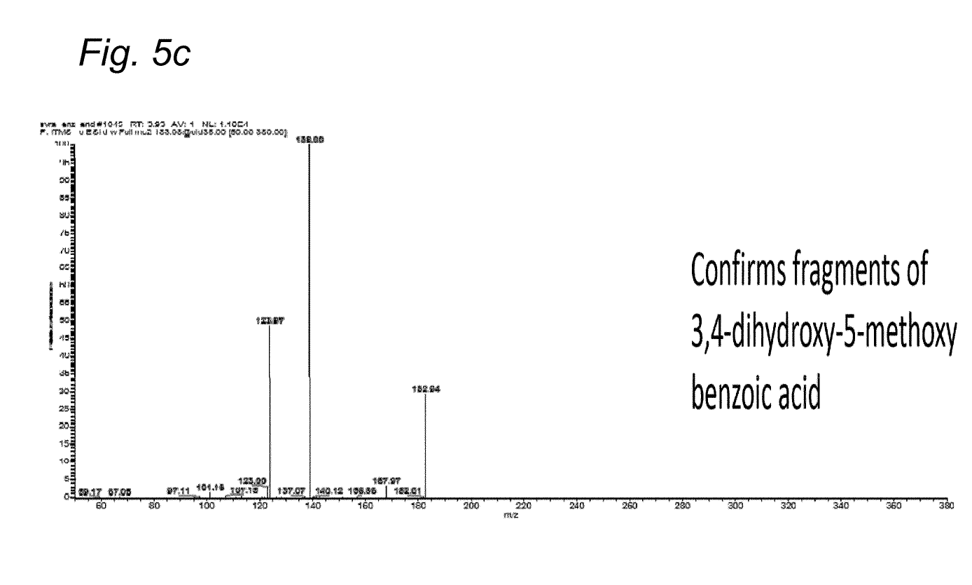

Syringic acid (12 mM, 2.38 g/L) was incubated with PPO7 from Example 2 (1.2 μM, 0.048 g/L, 2% loading (w/w)) in 50 mM BisTris buffer pH 6.5 with 10 μM CuSO₄. Blank prepared without the addition of enzyme was included. Incubations were performed in GC headspace vials with a total of 1 mL reaction volume in duplicate at room temperature for approximately 90 h. The reaction was stopped by adding 28 μL 1M HCl to lower the pH to ˜3. One of the replicates was analyzed by GC for the methanol formation. A calibration range of 0.23 to 11.6 mM methanol in buffer with HCl was included. The second replicate was analyzed using UHPLC and the formation 3,4-dihydroxy-5-methoxybenzoic acid was confirmed using MSMS (see FIG. 5). Table 1 shows the concentrations of syringic acid, methanol and 3,4-dihydroxy-5-methoxybenzoic acid. PPO7 catalyzed the demethylation of syringic acid, resulting in the formation of 3,4-dihydroxy-5-methoxybenzoic acid and methanol.

TABLE 1
Concentrations (mM) of syringic acid (UHPLC), methanol
(GC) and 3,4-dihydroxy-5-methoxybenzoic acid (UHPLC)

| | | | 3,4-dihydroxy-5- |
|---|---|---|---|
| | syringic acid | methanol | methoxybenzoic acid |
| | (mM) | (mM) | (mM) |
| Blank | 10.86 | 0 | 0 |
| PPO7 from Example 2 | 3.02 | 4.84 | 1.61 |

### Example 9 Demethylation of Lignin-Like Model Compounds, Lignin and Lignin-Derived Compounds by PPO7 Polypeptide Having Demethylating Activity

Model compounds syringic acid, syringol, 1,3-dimethoxybenzene and 3,5-dimethoxybenzoic acid (12 mM) were incubated with PPO7 from Example 2 (2% loading (w/w)) in 50 mM BisTris buffer pH 6.5 with 10 μM CuSO₄. For syringic acid and syringol PPO2 from Example 2 was included as a control. Lignin and lignin-derived compounds (10 g/L) were incubated with PPO7 from Example 2 (0.5 g/L, 5% loading (w/w)). PPO2 from Example 2 was included as a control and incubated using the same conditions. Blanks prepared without the addition of enzyme were included. Incubations were performed in GC headspace vials with a total of 1 mL reaction volume at 25° C. for approximately 48 h while shaking. The reaction was stopped by adding 28 μL 1M HCl to lower the pH to ˜3. Samples were analyzed by GC for methanol formation. A calibration range of 0.23 to 11.5 mM methanol in buffer with HCl was included. Table 2 shows the concentrations of methanol as determined by GC.

PPO7 catalyzed the demethylation of syringic acid and syringol, whereas PPO2 did not. Incubations on 1,3-dimethoxybenzene and 3,5-dimethoxybenzoic acid showed no methanol release for PPO7.

On lignin and lignin-derived compounds PPO7 showed an increased methanol concentration compared to the blank.

TABLE 2
Methanol concentration (mM) as determined by GC

| | methanol (mM) | | |
|---|---|---|---|
| | PPO7 | PPO2 | Blank |
| Model compounds | | | |
| Syringic acid | 4.58 | 0 | 0 |
| Syringol | 5.73 | 0.07 | 0.06 |
| 1,3-dimethoxybenzene | 0 | n.a. | 0 |
| 3,5-dimethoxybenzoic acid | 0 | n.a. | 0 |
| Lignin | | | |
| Wheat straw BioLignin | 0.28 | 0.09 | 0.10 |
| Corn stover BioLignin | 0.27 | 0.05 | 0 |
| Lignin-derived compounds | | | |
| Fractionated BioLignin | 0.46 | 0.36 | 0.28 |
| Purified wheat straw BioLignin | 0.21 | 0.15 | 0.05 |
| Lowest MW from lignin fractionation | 1.43 | 0.36 | 0.04 |
| Carboxymodified lignin (wheat)1 | 0.31 | 0 | 0 |
| Carboxymodified lignin (wheat)2 | 0.33 | 0 | 0 |
| Carboxymodified lignin (corn) | 0.35 | 0 | 0 |

### Example 10 Cloning, Preparation and Purification of Myceliophthora thermophila LPMO9B

DNA encoding LPMO9B (SEQ ID NO: 91; amino acid sequence: SEQ ID NO: 92; “mature” amino acid sequence without the signal sequence: SEQ ID NO: 93) was amplified from genomic DNA and cloned in pChi vector as previously described (WO 2010/107303 A2). Transformation of W1L#100.1Δpyr5ΔAlp1 strain was performed using the pyr5 marker and LPMO9B was prepared by fermentation of the W1L#100.1Δpyr5ΔAlp1 strain overexpressing the LPMO as described in WO 2010/107303 A2. Broth was harvested, filtrated to remove fungal biomass, concentrated and dialyzed against 10 mM potassium phosphate pH 7.0 plus 150 mM KCl using a 5 kDa PES membrane (Vivacell® 70, Sartorius). MtLPMO9B was purified in three subsequent chromatographic steps from the corresponding dialyzed enzyme preparations using an ÄKTA-Explorer preparative chromatography system (GE Healthcare, Uppsala, Sweden). The LPMO9B-containing enzyme preparation was loaded on a Source 30Q column (50 ml, GE Healthcare). A 20 mM potassium phosphate buffer (pH 7.8) was used to pre-equilibrate the column. Elution was performed using a linear gradient from 0 to 1 M NaCl in 20 mM potassium phosphate buffer (pH 7.8) at a flow rate of 10 mL min-1 and monitored at 220 and 280 nm. Fractions containing LPMO9B were pooled concentrated by ultrafiltration (Amicon Ultra, molecular mass cut-off of 3 kDa, Merck Millipore, Cork, Ireland) and subsequently loaded on a Source 30S column (50 mL, GE Healthcare). A 20 mM sodium acetate buffer (pH 5.0) was used to pre-equilibrate the column and elution was performed using a linear gradient from 0 to 1 M NaCl in 20 mM sodium acetate (pH 5.0) at a flow rate of 5 mL min-1. Peak fractions were pooled, concentrated and loaded on a Source 30S column (50 mL, GE Healthcare pre-equilibrated using a 10 mM sodium acetate buffer (pH 5.0)). The column was washed with 20 column volumes of starting buffer and subsequently eluted using a linear gradient from 0 to 1 M KCl in 20 mM sodium acetate (pH 5.0) at a flow rate of 5 mL min-1. Fractions containing the purified LPMO9B were pooled, concentrated as described above.

### Example 11 Regenerated Amorphous Cellulose (RAC Hydrolysis Assay with LPMO9B and PPO2, PPO7 or AbTyr in the Presence of Dimethoxyphenol Reducing Agent Sinapic Acid or Syringic Acid

Purification of Mushroom Tyrosinase AbTyr:

Crude mushroom tyrosinase was obtained from Sigma-Aldrich (St. Louis, Mo., USA) and purified as described previously (Kuijpers T F M, et al. (2014) Potato and Mushroom Polyphenol Oxidase Activities Are Differently Modulated by Natural Plant Extracts. Journal of Agricultural and Food Chemistry 62(1):214-221 and Kuijpers T F M, Gruppen H, Sforza S, van Berkel W J H, & Vincken J-P (2013) The antibrowning agent sulfite inactivates Agaricus bisporus tyrosinase through covalent modification of the copper-B site. FEBS Journal 280(23):6184-6195). The activity of the purified tyrosinase fraction was defined as the increase of A280 by 0.001 U per min by using L-tyrosine as a reference substrate. The determined activity of the fraction used in this work was 3000 U mL⁻¹. The protein concentration of this fraction was determined to be 270 μg/ml. Protein determination was determined using the BCA method.

Reducing agents sinapic acid and syringic acid were obtained from Sigma-Aldrich (St. Louis, Mo., USA). RAC was prepared from Avicel by swelling it in phosphoric acid, (Zhang Y H P, Cui J, Lynd L R, & Kuang L R (2006) A transition from cellulose swelling to cellulose dissolution by o-phosphoric acid: Evidence from enzymatic hydrolysis and supramolecular structure. Biomacromolecules 7(2):644-648.). RAC was dissolved in 50 mM phospate buffer (pH 5.0) to a final concentration of 1.5 mg/ml. Copper chloride was added to a final concentration of 2.5 μM and dimethoxy containg reducing agent sinapic acid or syringic acid to a final concentration of 2 mM. LPMO9B (5 μg/ml) with or without PPO2 (5 μg/ml), PPO7 (5 μg/ml) or AbTyr (7.5 U/ml) were added and incubated for 24 h at 50° C. in a head-over-tail rotator in portions of 1 mL total volume (Stuart® rotator, Bibby Scientific LTD, Stone, UK) at 20 rpm. Products were analysed by HPAEC and MALDI-TOF MS as described previously. (Frommhagen M, Sforza S, Westphal A H, Visser J, Hinz S W, Koetsier M J et al. Discovery of the combined oxidative cleavage of plant xylan and cellulose by a new fungal polysaccharide monooxygenase. Biotechnology for Biofuels. 2015; 8:101. doi:10.1186/s13068-015-0284-1). Results are shown in Table 3 (below). The Activity denoted as “Activity*” represents total release of non-oxidized and C1-oxidized gluco-oligosaccharides from RAC incubated with LPMO9B in the presence of sinapic or syringic acid as a reducing agent and with addition of either AbTyr, PPO7 or PPO2. Activity of LPMO9B equates to 100%, no activity was detected for LPMO9B in the absence of a reducing agent, all incubations were performed in duplicates. Standard deviations were less than 2%.

TABLE 3
RAC incubated with LPMO9B in the presence of dimethoxy
phenol reducing agent sinapic acid or syringic acid
with or without AbTyr, PPO7 or PPO2 addition

| | Activity* | Activity | Activity | Activity |
|---|---|---|---|---|
| Reducing Agent | LPMO9B | AbTyr | PPO7 | PPO2 |
| sinapic acid | 100 | 110 | 195 | 100 |
| syringic acid | 100 | 107 | 142 | 104 |

### Example 12 RAC Hydrolysis Assay with LPMO9B and PPO7 or AbTyr in the Presence of Phenolic Reducing Agents

Phenolic reducing agents were obtained from Sigma-Aldrich (St. Louis, Mo., USA). RAC was prepared as described above and dissolved in 50 mM phospate buffer (pH 5.0) to a final concentration of 1.5 mg/ml. Copper chloride was added to a final concentration of 2.5 μM and a phenolic reducing agent to a final concentration of 2 mM. LPMO9B (5 μg/ml) with or without PPO7 (5 μg/ml) or AbTyr (7.5 U/ml) were added and incubated for 24 h at 50° C. in a head-over-tail rotator in portions of 1 mL total volume (Stuart® rotator, Bibby Scientific LTD, Stone, UK) at 20 rpm. Products were analysed by HPAEC and MALDI-TOF MS as described previously. (Frommhagen M, Sforza S, Westphal A H, Visser J, Hinz S W, Koetsier M J et al. Discovery of the combined oxidative cleavage of plant xylan and cellulose by a new fungal polysaccharide monooxygenase. Biotechnology for Biofuels. 2015; 8:101. doi:10.1186/s13068-015-0284-1). Results are shown in Table 4 (below). The Activity denoted as Activity * represents total release of non-oxidized and C1-oxidized gluco-oligosaccharides from RAC incubated with LPMO9B in the presence of a phenolic reducing agent and with addition of either AbTyr or PPO7. Activity of LPMO9B equates to 100%, no activity was detected for LPMO9B in the absence of a reducing agent, all incubations were performed in duplicates. Standard deviations were less than 2%.

TABLE 4
RAC incubated with LPMO9B in the presence of phenolic
reducing agents with or without AbTyr or PPO7 addition

| | | Activity | Activity |
|---|---|---|---|
| | Activity* | MtLPMO9B | MtLPMO9B |
| Reducing agent | MtLPMO9B | AbTyr | MtPPO7 |
| 3-hydroxy-4- | 100 | 151 | 7558 |
| methoxycinnamic acid | | | |
| 4-hydroxy-3- | 100 | 117 | 131 |
| methoxyphenylactone | | | |
| coniferyl aldehyde | 100 | 128 | 562 |
| ferulic acid | 100 | 107 | 231 |
| guaiacol | 100 | 461 | 5143 |
| hesperidin | 100 | 56 | 771 |
| homovanillic acid | 100 | 156 | 580 |
| vanillic acid | 100 | 196 | 495 |
| 4-allyl-2,4- | 100 | 89 | 108 |
| methoxyphenol | | | |
| 3,4-dihydroxybenzoic | 100 | 99 | 139 |
| acid | | | |
| 3-methylcatechol | 100 | 58 | 104 |
| caffeic acid | 100 | 55 | 122 |
| 3,4-dihydroxy-5- | 100 | 99 | 22 |
| methoxybenzoic acid | | | |
| 3,4-dihydroxy-5- | 100 | 99 | 116 |
| methoxycinnamic acid | | | |
| gallic acid | 100 | 105 | 124 |

1. A polyphenoloxidase capable of demethylating an R-substituted mono- or di-methoxyphenol represented by 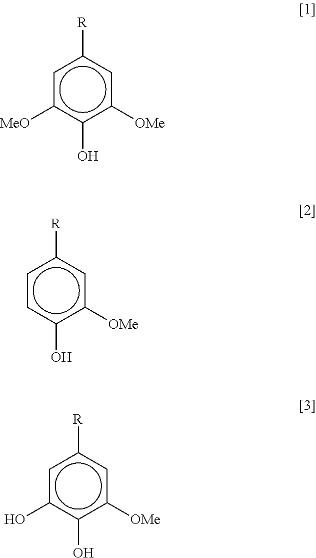
the above formula [1], [2] or [3], wherein R can be any single atom or chemical moiety, and wherein said polyphenoloxidase comprises or consists of a central tyrosinase domain.

2. A polyphenoloxisase according to claim 1 that comprises or consists of an amino acid sequence that is at least 70% identical to the amino acid sequence of at least one of SEQ ID NO: 37, 41 and 45.

3. A polyphenoloxisase according to claim 1 or 2 that is an enzyme that releases methanol from a R-substituted di-methoxyphenol represented by formula [1].

4. A polyphenoloxisase according to any one of the preceding claims that is obtainable from a fungus, preferably a Myceliophthora.

5. A polyphenoloxisase according to any one of the preceding claims that is obtainable from Myceliophthora thermophila C1.

6. A polyphenoloxisase according to any one of the preceding claims, wherein R is an organic moiety.

7. A method of demethylation of an R-substituted mono- or di-methoxyphenol represented by formula [1], [2], or [3], comprising the step of contacting a substrate comprising said R-substituted mono- or di-methoxyphenol with a polyphenoloxidase of any one of the claims 1-6, wherein R can be any single atom or chemical moiety.

8. A method according to claim 7, wherein said R-substituted mono- or di-methoxyphenol-comprising substrate further comprises or is contacted with 1-1000 μM of a copper salt, preferably selected from the group consisting of CuSO4, CuCl₂, CuBr₂, CuF₂, Cu(OH)₂ and Cu(NO₃)₂.

9. A method according to claim 7 or 8, wherein preferably said R-substituted mono- or di-methoxyphenol-comprising substrate has a pH between 5.0 and 8.0.

10. A method according to any one of claims 7-9, wherein said method is performed at a temperature between 20-60° C.

11. A method according to any one of claims 7-10, wherein said R-substituted mono- or di-methoxyphenol is an R-substituted di-methoxyphenol represented by formula [1], preferably syringic acid, sinapic acid, syringol, and/or a combination thereof.

12. A method according to any one of claims 7-11, wherein lignin is the source of said R-substituted mono- or di-methoxyphenol.

13. A process to increase the reactivity of lignin or lignin-comprising biomass saccharification mixture, wherein said process comprises the method step as defined in any one of claims 7-12.

14. A process for the conversion of lignin or lignin comprising biomass saccharification mixture to products such as bio fuel, macromolecules and/or aromatic chemicals, wherein said process comprises the method step as define in any one of claims 7-12.

15. A process for degrading and/or modifying (hemi-)cellulose in a (hemi-)cellulose-comprising substrate, wherein said process comprises the step of contacting said substrate with an accessory enzyme and a polyphenoloxidase capable of demethylating an R-substituted mono- or di-methoxyphenol represented by formula [1], [2], or [3] as defined in claim 1, wherein R can be any single atom or chemical moiety, optionally comprising the step of admixing an R-substituted mono- or di-methoxyphenol represented by formula [1], [2], or [3] wherein R can be any single atom or chemical moiety.

16. A process according to claim 15, wherein said accessory enzyme is a lytic polysaccharide monoxoygenases that comprises or consists of an amino acid sequence that is at least 70% identical to the amino acid sequence of SEQ ID NO: 1 or SEQ ID NO: 93.

17. A process of any one of claims 13-16, wherein said process is a process for saccharification, for degrading DDG, for baking, for preparing dough, for clarifying fruit, vegetable or cereal juice, for macerating vegetables or food and/or for increasing digestibility and/or nutritional properties of animal feedstocks or animal food.

18. An expression vector encoding both the polyphenoloxidase according to any one of claims 1-6, and optionally a lytic polysaccharide monoxoygenases as defined in claim 15 or 16.

19. A host cell recombinantly expressing the polyphenoloxidase according to any one of claims 1-6, optionally in combination with a lytic polysaccharide monoxoygenases as defined in claim 15 or 16 and/or transformed with the expression vector of claim 18.

20. A liquid, paste or solid formulation for use in a method or process according to any one of claims 7-17, wherein said formulation comprises the polyphenoloxidase according to any one of claims 1-6, and preferably further comprising one or more additional enzymes.

21. A liquid, paste or solid formulation of claim 20, further comprising the lytic polysaccharide monoxoygenases as defined in claim 15 or 16.

22. A composition comprising an R-substituted mono- or di-methoxyphenol-comprising substrate, wherein said composition further comprises the polyphenoloxidase according to any one of claims 1-6, an expression vector of claim 18, a host cell of claim 19, a liquid, paste or solid formulation according to claim 20 or 21 and/or a combination thereof, wherein said composition is preferably a biomass saccharification mixture, a baking composition or dough, animal feed or a feed premix, lignocellulosic material, paper and/or pulp.

23. Use of a polyphenoloxidase according to any one of claims 1-6, an expression vector according to claim 18, a host cell according to claim 19, a liquid, paste or solid formulation according to claim 20 or 21, and/or a composition according to claim 22, in a method or process of any one of claims 7-17.


SEQUENCE LISTING (93 sequences)

SEQ ID NO: 1 - protein, 208 aa, Myceliophthora thermophila
HYTLPRVGTGSDWQHVRRADNWQNNGFVGDVNSEQIRCFQATPAGAQDVYTVQAGSTVTYHANPSIYHPGPMQFYLARVPDGQDVKSWTGEGAVWFKVYEEQPQFGAQLTWPSNGKSSFEVPIPSCIRAGNYLLRAEHIALHVAQSQGGAQFYISCAQLQVTGGGSTEPSQKVSFPGAYKSTDPGILININYPVPTSYQNPGPAVFRC

SEQ ID NO: 2 - protein, 225 aa, Myceliophthora thermophila
MLTTTFALLTAALGVSAHYTLPRVGTGSDWQHVRRADNWQNNGFVGDVNSEQIRCFQATPAGAQDVYTVQAGSTVTYHANPSIYHPGPMQFYLARVPDGQDVKSWTGEGAVWFKVYEEQPQFGAQLTWPSNGKSSFEVPIPSCIRAGNYLLRAEHIALHVAQSQGGAQFYISCAQLQVTGGGSTEPSQKVSFPGAYKSTDPGILININYPVPTSYQNPGPAVFRC

SEQ ID NO: 3 - protein, 459 aa, Thielavia terrestris
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYISVSPRLYLLDSDGEYVMLKLNGQELSFDVDLSALPCGENGSLYLSQMDENGGANQYNTAGANYGSGYCDAQCPVQTWRNGTLNTSHQGFCCNEIDILEGNSRANALTPHSCTGTACDSAGCSFNPYGSGYKSYYGPGDTVDTSKTFTIITQFNTDNGSPSRNFVGITRKYQQNGVDIPSAQPGGDTISSCPSASAYGGLATMGKALSSGMVLVFSIRNDNSQYMNWLDSGNAGRCSSTEGNPSNILPNNPNTHFVFSNICWGDIGSTTNSTAPPPPPASSTTFSTTRRSSTTSSSPSCTQTHWGQCGAIGCSGCKTCTSGTTCQYSNDYYSQCL

SEQ ID NO: 4 - protein, 828 aa, Myceliophthora thermophila
MRTSSRLIGALAAALLPSALAQNNAPVTFTDPDSGITFNTWGLAEDSPQTKGGFTFGVALPSDALTTDAKEFIGYLKCARNDESGWCGVSLGGPMTNSLLIAAWPHEDTVYTSLRFATGYAMPDVYQGDAEITQVSSSVNSTHFSLIFRCENCLQWSQSGATGGASTSNGVLVLGWVQAFADPGNPTCPDQITLEQHDNGMGIWGAQLNSDAASPSYTEWAAQATKTVTGDCGGPTETSVVGVPVPTGVSFDYIVVGGGAGGIPAADKLSEAGKSVLLIEKGFASTANTGGTLGPEWLEGHDLTRFDVPGLCNQIWVDSKGIACEDTDQMAGCVLGGGTAVNAGLWFKPYSLDWDYLFPSGWKYKDVQPAINRALSRIPGTDAPSTDGKRYYQQGFDVLSKGLAGGGWTSVTANNAPDKKNRTFSHAPFMFAGGERNGPLGTYFQTAKKRSNFKLWLNTSVKRVIRQGGHITGVEVEPFRDGGYQGIVPVTKVTGRVILSAGTFGSAKILLRSGIGPNDQLQVVAASEKDGPTMISNSSWINLPVGYNLDDHLNTDTVISHPDVVFYDFYEAWDNPIQSDKDSYLNSRTGILAQAAPNIGPMFWEEIKGADGIVRQLQWTARVEGSLGAPNGKTMTMSQYLGRGATSRGRMTITPSLTTVVSDVPYLKDPNDKEAVIQGIINLQNALKNVANLTWLFPNSTITPRQYVDSMVVSPSNRRSNHWMGTNKIGTDDGRKGGSAVVDLNTKVYGTDNLFVIDASIFPGVPTTNPTSYIVTASEHASARILALPDLTPVPKYGQCGGREWSGSFVCADGSTCQMQNEWYSQCL

SEQ ID NO: 5 - protein, 787 aa, Myceliophthora thermophila
MKLLSRVGATALAATLSLQQCAAQMTEGTYTDEATGIQFKTWTASEGAPFTFGLTLPADALEKDATEYIGLLRCQITDPASPSWCGISHGQSGQMTQALLLVAWASEDTVYTSFRYATGYTLPGLYTGDAKLTQISSSVSEDSFEVLFRCENCFSWDQDGTKGNVSTSNGNLVLGRAAAKDGVTGPTCPDTAEFGFHDNGFGQWGAVLEGATSDSYEEWAKLATTTPETTCDGTGPGDKECVPAPEDTYDYIVVGAGAGGITVADKLSEAGHKVLLIEKGPPSTGLWNGTMKPEWLESTDLTRFDVPGLCNQIWVDSAGIACTDTDQMAGCVLGGGTAVNAGLWWKPHPADWDENFPEGWKSSDLADATERVFKRIPGTSHPSQDGKLYRQEGFEVISKGLANAGWKEISANEAPSEKNHTYAHTEFMFSGGERGGPLATYLASAAERSNFNLWLNTAVRRAVRSGSKVTGVELECLTDGGFSGTVNLNEGGGVIFSAGAFGSAKLLLRSGIGPEDQLEIVASSKDGETFTPKDEWINLPVGHNLIDHLNTDLIITHPDVVFYDFYAAWDEPITEDKEAYLNSRSGILAQAAPNIGPMMWDQVTPSDGITRQFQWTCRVEGDSSKTNSTHAMTLSQYLGRGVVSRGRMGITSGLSTTVAEHPYLHNNGDLEAVIQGIQNVVDALSQVADLEWVLPPPDGTVADYVNSLIVSPANRRANHWMGTAKLGTDDGRSGGTSVVDLDTKVYGTDNLFVVDASVFPGMSTGNPSAMIVIVAEQAAQRILALRS

SEQ ID NO: 6 - protein, 577 aa, Myceliophthora thermophila
MQVASKLVAVTGGALALWLHPVAAQEGCTNISSTETYDYIVVGSGAGGIPVADRLSEAGHKVLLIEKGPPSTGRWGGIMKPEWLIGTNLTRFDVPGLCNQIWADPTGAICTDVDQMAGCMLGGGTAVNAGLWWKPHPADWDVNFPEGWHSEDMAEATERVFERIPGTITPSMDGKRYLSQGFDMLGGSLEAAGWEYLVPNEHPDRKNRTYGHSTFMYSGGERGGPLATYLVSAVQREGFTLWMNTTVTRIIREGGHATGVEVQCSNSEAGQAGIVPLTPKTGRVIVSAGAFGSAKLLFRSGIGPKDQLNIVKNSTDGPSMISEDQWIELPVGYNLNDHVGTDIEIAHPDVVFYDYYGAWDEPIVEDTERYVANRTGPLAQAAPNIGPIFWETIKGSDGVSRHLQWQARVEGKLNTSMTITQYLGTGSRSRGRMTITRRLNTVVSTPPYLRDEYDREAVIQGIANLRESLKGVANLTWITPPSNVTVEDFVDSIPATPARRCSNHWIGTAKIGLDDGREGGTSVVDLNTKVYGTDNIFVVDASIFPGHITGNPSAAIVIAAEYAAAKILALPAPEDAA

SEQ ID NO: 7 - DNA, 675 bp, Artificial
  cDNA encoding MtLPMO9A
atgctgacaacaaccttcgccctcctgacggccgctctcggcgtcagcgcccattataccctccccagggtcgggaccggttccgactggcagcacgtgcggcgggctgacaactggcaaaacaacggcttcgtcggcgacgtcaactcggagcagatcaggtgcttccaggcgacccctgccggcgcccaagacgtctacactgttcaggcgggatcgaccgtgacctaccacgccaaccccagtatctaccaccccggccccatgcagttctacctggcccgcgttccggacggacaggacgtcaagtcgtggaccggcgagggtgccgtgtggttcaaggtgtacgaggagcagcctcaatttggcgcccagctgacctggcctagcaacggcaagagctcgttcgaggttcctatccccagctgcattcgggcgggcaactacctcctccgcgctgagcacatcgccctgcacgttgcccaaagccagggcggcgcccagttctacatctcgtgcgcccagctccaggtcactggtggcggcagcaccgagccttctcagaaggtttccttcccgggtgcctacaagtccaccgaccccggcattcttatcaacatcaactaccccgtccctacctcgtaccagaatccgggtccggctgtcttccgttgc

SEQ ID NO: 8 - DNA, 963 bp, Myceliophthora thermophila
agcggcgttctgaaaaggaggaacagaactaacgaagacgacccaagcataccaaggtcgtgttgattagcacccccgattcttccagttctttccgccatctgcaatgctgacaacaaccttcgccctcctgacggccgctctcggcgtcagcgcccattataccctccccagggtcgggaccggttccgactggcagcacgtgcggcgggctgacaactggcaaaacaacggcttcgtcggcgacgtcaactcggagcagatcaggtgcttccaggcgacccctgccggcgcccaagacgtctacactgttcaggcgggatcgaccgtgacctaccacgccaaccccagtatctaccaccccggccccatgcagttctacctggcccgcgttccggacggacaggacgtcaagtcgtggaccggcgagggtgccgtgtggttcaaggtgtacgaggagcagcctcaatttggcgcccagctgacctggcctagcaacggtgcgttgatcattttccttcttcttccttctttcttccgttgcatatgctaactgttctcttgcttgcaggcaagagctcgttcgaggttcctatccccagctgcattcgggcgggcaactacctcctccgcgctgagcacatcgccctgcacgttgcccaaagccagggcggcgcccagttctacatctcgtgcgcccagctccaggtcactggtggcggcagcaccgagccttctcagaaggtttccttcccgggtgcctacaagtccaccgaccccggcattcttatcaacatcaactaccccgtccctacctcgtaccagaatccgggtccggctgtcttccgttgctaagcgggcacaacaagggaattggaacggggcaatgggggcgggctggagccgcctgcgtccttgttttcttaccctcgccgttcgtacgaatcagggccaagcaaaaca

SEQ ID NO: 9 - DNA, 1380 bp, Artificial
  cDNA encoding TvEG I
atggcgccctcagttacactgccgttgaccacggccatcctggccattgcccggctcgtcgccgcccagcaaccgggtaccagcacccccgaggtccatcccaagttgacaacctacaagtgtacaaagtccggggggtgcgtggcccaggacacctcggtggtccttgactggaactaccgctggatgcacgacgcaaactacaactcgtgcaccgtcaacggcggcgtcaacaccacgctctgccccgacgaggcgacctgtggcaagaactgcttcatcgagggcgtcgactacgccgcctcgggcgtcacgacctcgggcagcagcctcaccatgaaccagtacatgcccagcagctctggcggctacatcagcgtctctcctcggctgtatctcctggactctgacggtgagtacgtgatgctgaagctcaacggccaggagctgagcttcgacgtcgacctctctgctctgccgtgtggagagaacggctcgctctacctgtctcagatggacgagaacgggggcgccaaccagtataacacggccggtgccaactacgggagcggctactgcgatgctcagtgccccgtccagacatggaggaacggcaccctcaacactagccaccagggcttctgctgcaacgagattgatatcctggagggcaactcgagggcgaatgccttgacccctcactcttgcacgggcacggcctgcgactctgccggttgcagcttcaacccctatggcagcggctacaaaagctactacggccccggagataccgttgacacctccaagaccttcaccatcatcacccagttcaacacggacaacggctcgccctcgcgcaactttgtgggcatcacccgcaagtaccagcaaaacggcgtcgacatccccagcgcccagcccggcggtgacaccatctcgtcctgcccgtccgcctcagcctacggcggcctcgccaccatgggcaaggccctgagcagcggcatggtgctcgtgttcagcattcggaacgacaacagccagtacatgaactggctcgacagcggcaacgccggccgctgcagcagcaccgagggcaacccatccaacatcctgcccaacaaccccaacacgcacttcgtcttctccaacatctgctggggagacattgggtctactacgaactcgactgcgcccccgcccccgcctgcgtctagcacgacgttctcgactacacggaggagctcgacgacttcgagcagcccgagctgcacgcagactcactgggggcagtgcggtgccattgggtgcagcgggtgcaagacgtgcacgtcgggcactacgtgccagtatagcaacgactactactcgcaatgcctttag

SEQ ID NO: 10 - protein, 337 aa, Myceliophthora thermophila
MKFLDVLLGAAAASSALAAPTCTTKTKRAGKFKFVGVNQSCAEFGQDTLPGQLNKHYTWPAKSSIDTLLATGMNTIRIPFMMERLIPNQLTGTVNETYSAGLIDTVSYVTSKGAYAVIDPHNFGRYYTQVITDVEGFKAWWTTTAGLFADNDKVIFDTNNEYHDMDQSLVVNLNQAAIDGIRAAGATSQYIFIEGNSWTGAWTWVSSGNGESLLNLSDPEGDDKLIYEMHQYLDEDGSGTHEQCVSGTIGRERLQAATEWLKANGKKAILGETAGGANDQCISALTGMLSFMEENSDVWQGWLWWAAGPWWADYMYSIEPPSGTAYTKVLPSLQPYI

SEQ ID NO: 11 - DNA, 1014 bp, Artificial
  cDNA encoding MtEG VIII
atgaagttcctcgacgtccttctcggtgccgcagctgccagctcggccctggcagcgccgacttgcacaaccaagacaaagcgcgcgggcaagttcaagtttgtcggcgtcaaccagtcctgcgccgagtttggccaggacacgctcccgggccagctcaacaagcactacacctggccggccaagtcgagcattgatacactcctcgccaccggaatgaacaccattcgcatcccgtttatgatggagcgtcttatccccaaccaactgacgggaaccgtcaacgaaacgtactccgcggggcttatcgataccgtttcctacgtcacgagcaagggagcctatgctgtcattgacccccataacttcggccggtactacacgcaagtcatcaccgacgtcgaaggcttcaaggcctggtggacgaccacggccgggctgtttgccgacaacgacaaggtcatcttcgacaccaacaacgagtaccacgacatggaccaatcgctcgttgtcaatctgaaccaggccgccatcgacggcatccgggccgccggcgccacctcgcagtacatctttatcgagggcaactcgtggacgggcgcatggacctgggtctcctcgggcaacggcgagtccctgctgaacctgtcggacccggagggcgacgacaagctcatctacgagatgcaccagtacctggacgaggacgggtcgggcacgcacgagcagtgcgtctcgggcaccatcggccgggagcgcctccaggcggcgaccgagtggctcaaggccaacggcaagaaggccatcctgggcgagacggccggcggcgccaacgaccagtgcatctcggccctgaccggcatgctctccttcatggaggagaactcggacgtctggcagggctggctctggtgggctgccggtccctggtgggccgactacatgtactcgattgagccgccgagcggtaccgcgtacaccaaggttttgcccagcctccagccttatatctga

SEQ ID NO: 12 - DNA, 2000 bp, Myceliophthora thermophila
gccccggggcgcgttccgagttgaatgaatgagcgggttaatatagtaaagatctgcgctataacctggtctcgagcgcggagccaacctttccgagcacgcatcttgataatgagtaaacccccttcaacaaacttgtgaaagagctacctatagctggacatataaatctgcaatggccatctcagaaagggaccccatgcggaaccgttggtgcagcaggaagcggtttactttctgtcgacaaccaactcaaccatgaagttcctcgacgtccttctcggtgccgcagctgccagctcggccctggcagcgccgacttgcacaaccaagacaaagcgcgcgggcaagttcaagtttgtcggcgtcaaccagtcctgcgccgagtttggccaggacacgctcccgggccagctcaacaagcactacacctggccggccaagtcgagcattgatgtaagtgtctttgaacttcgagctaccatgacgaaaaaggaacaatggaagtaaccaggctttcgaaaatcagacactcctcgccaccggaatgaacaccattcgcatcccgtttatgatgtaggactgagtgccctttccgcttcgctttacaagtaaagagggcacacattgctgacaacgggttgcagggagcgtcttatccccaaccaactgacgggaaccgtcaacgaaacgtactccgcggggcttatcgatgtatgaaacagacatagagcttctcaagaacggggggccggaatttgctgaccggaatttcagaccgtttcctacgtcacgagcaagggagcctatgctgtcattgacccccataacttcggtacgaccagcttacaatgaccatgtcttgggaccgtggcagacaagctaaccaagacttgctccaccaaatttcacaggccggtactacacgcaagtcatcaccgacgtcgaaggcttcaaggcctggtggacgaccacggccgggctgtttgccgacaacgacaaggtcatcttcgacaccaacaacgagtaccacgacatggaccaatcgctcgttgtcaatctgaaccaggccgccatcgacggcatccgggccgccggcgccacctcgcagtacatctttatcgagggcaactcgtggacgggcgcatggacctgggtacgaacacggatttctgatccggatatttatattgattttatgattttgtttttttggaccggtgtaatagcagctgacaatcatgtcatttccaggtctcctcgggcaacggcgagtccctgctgaacctgtcggacccggagggcgacgacaagctcatctacgagatgcaccagtacctggacgaggacgggtcgggcacgcacgagcagtgcgtctcgggcaccatcggccgggagcgcctccaggcggcgaccgagtggctcaaggccaacggcaagaaggccatcctgggcgagacggccggcggcgccaacgaccagtgcatctcggccctgaccggcatgctctccttcatggaggagaactcggacgtctggcagggctggctctggtgggctgccggtccctggtgggccgactacatgtactcgatgtgagtgttttcttatttttcttctctcatttcattcctttctgaagatcttgtttttttttccctccctgtctaacctcgcttagtgagccgccgagcggtaccgcgtacaccaaggttttgcccagcctccagccttatatctgagctcggtcggttctgcaaacgttgcggagcggaagaagattccactgttggggttgatcacaaagatactgagccttaagtgtactttacttcatcagggtagatgaaatcgagcaagatacatacagttccctgagagctatggtccttacgtcacgtcactcaactgaggacccaaattgcccgagtcttaatccaaagagtactccgtaccgctaccagggcgatgactcctgtaaattacactgataatacaaa

SEQ ID NO: 13 - protein, 389 aa, Myceliophthora thermophila
MKSSILASVFATGAVAQSGPWQQCGGIGWQGSTDCVSGYHCVYQNDWYSQCVPGAASTTLQTSTTSRPTATSTAPPSSTTSPSKGKLKWLGSNESGAEFGEGNYPGLWGKHFIFPSTSAIQTLINDGYNIFRIDFSMERLVPNQLTSSFDEGYLRNLTEVVNFVTNAGKYAVLDPHNYGRYYGNVITDTNAFRTFWTNLAKQFASNSLVIFDTNNEYNTMDQTLVLNLNQAAIDGIRAAGATSQYIFVEGNAWSGAWSWNTTNTNMAALTDPQNKIVYEMHQYLDSDSSGTHAECVSSNIGAQRVVGATQWLRANGKLGVLGEFAGGANAVCQQAVTGLLDHLQDNSDVWLGALWWAAGPWWGDYMYSFEPPSGTGYVNYNSILKKYLP

SEQ ID NO: 14 - protein, 373 aa, Myceliophthora thermophila
QSGPWQQCGGIGWQGSTDCVSGYHCVYQNDWYSQCVPGAASTTLQTSTTSRPTATSTAPPSSTTSPSKGKLKWLGSNESGAEFGEGNYPGLWGKHFIFPSTSAIQTLINDGYNIFRIDFSMERLVPNQLTSSFDEGYLRNLTEVVNFVTNAGKYAVLDPHNYGRYYGNVITDTNAFRTFWTNLAKQFASNSLVIFDTNNEYNTMDQTLVLNLNQAAIDGIRAAGATSQYIFVEGNAWSGAWSWNTTNTNMAALTDPQNKIVYEMHQYLDSDSSGTHAECVSSNIGAQRVVGATQWLRANGKLGVLGEFAGGANAVCQQAVTGLLDHLQDNSDVWLGALWWAAGPWWGDYMYSFEPPSGTGYVNYNSILKKYLP

SEQ ID NO: 15 - DNA, 1170 bp, Artificial
  cDNA encoding MtEG II
atgaagtcctccatcctcgccagcgtcttcgccacgggcgccgtggctcaaagtggtccgtggcagcaatgtggtggcatcggatggcaaggatcgaccgactgtgtgtcgggttaccactgcgtctaccagaacgattggtacagccagtgcgtgcctggcgcggcgtcgacaacgctccagacatctaccacgtccaggcccaccgccaccagcaccgcccctccgtcgtccaccacctcgcctagcaagggcaagctcaagtggctcggcagcaacgagtcgggcgccgagttcggggagggcaactaccccggcctctggggcaagcacttcatcttcccgtcgacttcggcgattcagacgctcatcaatgatggatacaacatcttccggatcgacttctcgatggagcgtctggtgcccaaccagttgacgtcgtccttcgacgagggctacctccgcaacctgaccgaggtggtcaacttcgtgacgaacgcgggcaagtacgccgtcctggacccgcacaactacggccggtactacggcaacgtcatcacggacacgaacgcgttccggaccttctggaccaacctggccaagcagttcgcctccaactcgctcgtcatcttcgacaccaacaacgagtacaacacgatggaccagaccctggtgctcaacctcaaccaggccgccatcgacggcatccgggccgccggcgcgacctcgcagtacatcttcgtcgagggcaacgcgtggagcggggcctggagctggaacacgaccaacaccaacatggccgccctgacggacccgcagaacaagatcgtgtacgagatgcaccagtacctcgactcggacagctcgggcacccacgccgagtgcgtcagcagcaacatcggcgcccagcgcgtcgtcggagccacccagtggctccgcgccaacggcaagctcggcgtcctcggcgagttcgccggcggcgccaacgccgtctgccagcaggccgtcaccggcctcctcgaccacctccaggacaacagcgacgtctggctgggtgccctctggtgggccgccggtccctggtggggcgactacatgtactcgttcgagcctccttcgggcaccggctatgtcaactacaactcgatcctaaagaagtacttgccgtaa

SEQ ID NO: 16 - DNA, 1616 bp, Myceliophthora thermophila
atgaagtcctccatcctcgccagcgtcttcgccacgggcgccgtggctcaaagtggtccgtggcagcaatgtggtggcatcggatggcaaggatcgaccgactgtgtgtcgggttaccactgcgtctaccagaacgattggtacagccagtgcgtgcctggcgcggcgtcgacaacgctccagacatctaccacgtccaggcccaccgccaccagcaccgcccctccgtcgtccaccacctcgcctagcaagggcaagctcaagtggctcggcagcaacgagtcgggcgccgagttcggggagggcaactaccccggcctctggggcaagcacttcatcttcccgtcgacttcggcgattcaggtacggccaataataatatattattatagcaggcaggagggagcaggagaagaagggaggggcaggtggccaacaatcggaagaagaccgggaggcactgaccgttgattcctttgtgtaatagacgctcatcaatgatggatacaacatcttccggatcgacttctcgatggagcgtctggtgcccaaccagttgacgtcgtccttcgacgagggctacctccgcaacctgaccgaggtggtcaacttcgtgacgaacgcgggcaagtacgccgtcctggacccgcacaactacggccggtactacggcaacgtcatcacggacacgaacgcgttccggaccttctggaccaacctggccaagcagttcgcctccaactcgctcgtcatcttcgacaccaacaacgagtacaacacgatggaccagaccctggtgctcaacctcaaccaggccgccatcgacggcatccgggccgccggcgcgacctcgcagtacatcttcgtcgagggcaacgcgtggagcggggcctggagctggaacacgaccaacaccaacatggccgccctgacggacccgcagaacaagatcgtgtacgagatgcaccagtacctcgactcggacagctcgggcacccacgccgagtgcgtcagcagcaacatcggcgcccagcgcgtcgtcggagccacccagtggctccgcgccaacggcaagctcggcgtcctcggcgagttcgccggcggcgccaacgccgtctgccagcaggccgtcaccggcctcctcgaccacctccaggacaacagcgacgtctggctgggtgccctctggtgggccgccggtccctggtggggcgactacatgtactcgttcggtaagtttctcccttgttcttggctttccccccagtaagggagtcaggcaacatgcccaagaccggctcggcttcgcttcaaggcgttcgttgtacacactgaagagttccaacttccaaccctgttcgtgtcctccgatcagcttcgacggggtgaagggggaagggatttgggagtgaggtggaggtcaaaaggagggatatccccagatctccacaaacggccctgagccaacaacagcctctggggtcaaaatgggcgccaaccatacggtcattcactcaggacacctgctaacgcgtctcttttttttgtttccagagcctccttcgggcaccggctatgtcaactacaactcgatcctaaagaagtacttgccgtaa

SEQ ID NO: 17 - protein, 456 aa, Myceliophthora thermophila
MGRGAAFLGLASLLVGAAKAQTPGEGEEVHPQITTYRCTKADGCEEKTNYIVLDALSHPVHQVDNPYNCGDWGQKPNETACPDLESCARNCIMDPVSDYGRHGVSTDGTSLRLKQLVGGNVVSPRVYLLDETKERYEMLKLTGNEFTFDVDATKLPCGMNSALYLSEMDATGARSELNPGGATFGTGYCDAQCYVTPFINGLGNIEGKGACCNEMDIWEANARAQHIAPHPCSKAGPYLCEGAECEFDGVCDKNGCAWNPYRVNVTDYYGEGAEFRVDTTRPFSVVTQFRAGGDAGGGKLESIYRLFVQDGRVIESYVVDKPGLPPTDRMTDEFCAATGAARFTELGAMEAMGDALTRGMVLALSIWWSEGDNMNWLDSGEAGPCDPDEGNPSNIIRVQPDPEVVFSNLRWGEIGSTYESAVDGPVGKGKGKGKGKAPAGDGNGKEKSNGKRFRRF

SEQ ID NO: 18 - protein, 436 aa, Myceliophthora thermophila
QTPGEGEEVHPQITTYRCTKADGCEEKTNYIVLDALSHPVHQVDNPYNCGDWGQKPNETACPDLESCARNCIMDPVSDYGRHGVSTDGTSLRLKQLVGGNVVSPRVYLLDETKERYEMLKLTGNEFTFDVDATKLPCGMNSALYLSEMDATGARSELNPGGATFGTGYCDAQCYVTPFINGLGNIEGKGACCNEMDIWEANARAQHIAPHPCSKAGPYLCEGAECEFDGVCDKNGCAWNPYRVNVTDYYGEGAEFRVDTTRPFSVVTQFRAGGDAGGGKLESIYRLFVQDGRVIESYVVDKPGLPPTDRMTDEFCAATGAARFTELGAMEAMGDALTRGMVLALSIWWSEGDNMNWLDSGEAGPCDPDEGNPSNIIRVQPDPEVVFSNLRWGEIGSTYESAVDGPVGKGKGKGKGKAPAGDGNGKEKSNGKRFRRF

SEQ ID NO: 19 - DNA, 1368 bp, Artificial
  cDNA encoding MtEG I
atgggtcgcggcgctgctttcctaggcctcgcctcgctcctcgtgggcgcggccaaggcccagacgcccggcgagggcgaggaggtgcacccgcagatcacgacgtaccgctgcaccaaggcggacgggtgcgaggagaagaccaactacatcgtgctggacgccctatcgcacccggtccaccaggtcgacaacccgtacaactgcggcgactggggccagaagcccaacgagacggcctgcccggacctcgagtcgtgcgccaggaactgcatcatggacccggtctcggactacggccggcacggggtctcgaccgacggcacctcgctgcgcctcaagcagctagtcggcggcaacgtcgtcagcccgcgcgtctacctgctcgacgagaccaaggagcgctacgagatgctcaagctgaccggcaacgagttcacctttgacgtcgacgccaccaagctgccctgcggcatgaacagcgccctctacctctccgagatggacgccaccggcgcccggagcgagctcaacccgggcggcgccacctttggcaccggctactgcgacgcccagtgctacgtcacccccttcatcaacggcctcggcaacatcgagggcaagggcgcgtgctgcaacgagatggatatctgggaggccaacgcgcgggcgcagcacatcgcgccgcacccgtgcagcaaggcggggccgtacctgtgcgagggcgccgagtgcgagttcgacggcgtgtgcgacaagaacggctgcgcctggaacccgtaccgggtcaacgtgacggactactacggcgagggcgccgagttcagggtggacacgacccggcccttctcggtcgtcacgcagttccgcgccggcggcgacgcggggggcggcaagctcgagagcatctaccggctcttcgtccaggacggcagggtgatcgagtcgtacgtcgtcgacaagcccggcctgcccccgacggaccgcatgacggacgagttctgcgccgccaccggcgccgcccgcttcacggagctcggcgccatggaggccatgggcgacgccctgacgcgcggcatggtcctcgccctcagcatctggtggagcgagggcgacaacatgaactggctcgactcgggcgaggccggcccctgcgacccggacgagggcaacccgtccaacatcatccgcgtccagcccgacccggaggtcgtcttcagcaacctgcgctggggcgagatcggctcaacctacgagtccgccgtcgacgggcccgtcggcaagggcaagggcaagggcaagggcaaggctcccgccggcgacggcaacgggaaggagaagagcaatggcaagcgcttcaggaggttc

SEQ ID NO: 20 - DNA, 2507 bp, Myceliophthora thermophila
ctatataaaggtacttacagataaaatccccaggttgacagccctttcgagcataagaatccaggtcctatagtggccggctcgaagcccagatttgagccctattgagaataactatatggcactggatgaaggattggcttgaacatcgctacgatgtttaatcgctgagtcttacagggcttcgactggctgttctggaggcgtgggaggcagtcccccccgagtttcttcagtgcctggcacactcaatgcctcgacggctagcaaaaaccaggggggagagactgggtattgatacttctgtattatagaaatttgtattgggttggttgtacctagacttcttggctacttttctgatttgcgttagttgtggggcatgtatcgcactaggattaataatgctggaagaggtgtagagatgttgcgtcgcatggccaccgagatccggcctggcgtcgtgccttgcagctcgcacgatgcgcgcgacgatgcgggacacccctcccccctcccccctcccccctacattaggaggggatccggggccgctgcccgttttccgtcttggtattcccggctgacacaacggttctccagagtgataaccgaggcacaccagtagcagtccacgatggcacggcaggggatacattccgcccggaacaacccaagatctgggaatcccagtgacgaccccggggtcctccggggtcccccgatttcgtctgaaccgacgaactggaaggaggcgagccttggaacgatgggggatatgatatatattaagatgcaacattctctccctccctcctctctctctccctccccccctctctcctcctcttcccctcctccccgccgttctctctccatgagcctcaattcttgctccgagcgcggtgtatttcccccacgaggaattgacaaaagaaaaagaaaaagacaagactctcgagaacgatgggtcgcggcgctgctttcctaggcctcgcctcgctcctcgtgggcgcggccaaggcccagacgcccggcgagggcgaggaggtgcacccgcagatcacgacgtaccgctgcaccaaggcggacgggtgcgaggagaagaccaactacatcgtgctggacgccctatcgcacccggtccaccaggtcgacaacccgtacaactgcggcgactggggccagaagcccaacgagacggcctgcccggacctcgagtcgtgcgccaggaactgcatcatggacccggtctcggactacggccggcacggggtctcgaccgacggcacctcgctgcgcctcaagcagctagtcggcggcaacgtcgtcagcccgcgcgtctacctgctcgacgagaccaaggagcgctacgagatgctcaagctgaccggcaacgagttcacctttgacgtcgacgccaccaagctgccctgcggcatgaacagcgccctctacctctccgagatggacgccaccggcgcccggagcgagctcaacccgggcggcgccacctttggcaccggctactgcgacgcccagtgctacgtcacccccttcatcaacggcctcgtacgtattctcctattaatcttgtttcttttttccttttctttttttgatataatagaattaagaagaactcggtgggctgacatgacacagggcaacatcgagggcaagggcgcgtgctgcaacgagatggatatctgggaggccaacgcgcgggcgcagcacatcgcgccgcacccgtgcagcaaggcggggccgtacctgtgcgagggcgccgagtgcgagttcgacggcgtgtgcgacaagaacggctgcgcctggaacccgtaccgggtcaacgtgacggactactacggcgagggcgccgagttcagggtggacacgacccggcccttctcggtcgtcacgcagttccgcgccggcggcgacgcggggggcggcaagctcgagagcatctaccggctcttcgtccaggacggcagggtgatcgagtcgtacgtcgtcgacaagcccggcctgcccccgacggaccgcatgacggacgagttctgcgccgccaccggcgccgcccgcttcacggagctcggcgccatggaggccatgggcgacgccctgacgcgcggcatggtcctcgccctcagcatctggtggagcgagggcgacaacatgaactggctcgactcgggcgaggccggcccctgcgacccggacgagggcaacccgtccaacatcatccgcgtccagcccgacccggaggtcgtcttcagcaacctgcgctggggcgagatcggctcaacctacgagtccgccgtcgacgggcccgtcggcaagggcaagggcaagggcaagggcaaggctcccgccggcgacggcaacgggaaggagaagagcaatggcaagcgcttcaggaggttctgagcaaccttgatattatttttttctttctttccttcaccagttaattagttgcctttgattagaaagagagagagaaa

SEQ ID NO: 21 - protein, 247 aa, Myceliophthora thermophila
MQPFLLLFLSSVTAASPLTALDKRQQATLCEQYGYWSGNGYEVNNNNWGKDSASGGHQCTYVDSSSSSGVAWHTTWQWEGGQNQVKSFANCGLQVPKGRTISSISNLQTSISWSYSNTNIRANVAYDLFTAADPNHATSSGDYELMIWLARFGDVYPIGSSQGHVNVAGQDWELWTGFNGNMRVYSFVAPSPRNSFSANVKDFFNYLQSNQGFPASSQYLLIFQAGTEPFTGGETTLTVNNYSARVA

SEQ ID NO: 22 - protein, 232 aa, Myceliophthora thermophila
SPLTALDKRQQATLCEQYGYWSGNGYEVNNNNWGKDSASGGHQCTYVDSSSSSGVAWHTTWQWEGGQNQVKSFANCGLQVPKGRTISSISNLQTSISWSYSNTNIRANVAYDLFTAADPNHATSSGDYELMIWLARFGDVYPIGSSQGHVNVAGQDWELWTGFNGNMRVYSFVAPSPRNSFSANVKDFFNYLQSNQGFPASSQYLLIFQAGTEPFTGGETTLTVNNYSARVA

SEQ ID NO: 23 - DNA, 741 bp, Artificial
  cDNA encoding MtEG III
atgcagccgtttctgctcttgttcctctcgtcggtcacggcggcgagccccctgacggcgctcgacaagcggcagcaggcgacgttgtgcgagcagtacggctactggtcgggcaacggttacgaggtcaacaacaacaactggggcaaggattcggcctcgggcggccatcagtgcacctacgtcgacagcagcagctccagcggcgtcgcctggcacacgacctggcagtgggaaggaggccagaaccaggtcaagagcttcgccaactgcggcctgcaggtgcccaagggcaggaccatctcgtccatcagcaacctgcagacctccatctcgtggtcctacagcaacaccaacatccgcgccaacgtggcctacgacctcttcaccgcggcagacccgaaccacgcgaccagcagcggcgactacgagctcatgatctggctggcgagattcggcgacgtctaccccatcggctcgtcccagggccacgtcaacgtggccggccaggactgggagctgtggacgggcttcaacggcaacatgcgggtctacagcttcgtagcgcccagcccccgcaacagcttcagcgccaacgtcaaggacttcttcaactatctccagtccaaccagggcttcccggccagcagccaataccttctcatcttccaggcgggcaccgagcccttcaccggcggcgagaccacccttaccgtcaacaactactctgcaagggttgct

SEQ ID NO: 24 - DNA, 1305 bp, Myceliophthora thermophila
cgacaaagacccgtcagtgattaataataattagtagcagtttctttctttcaagactcaagaatactcctttccgccatcgtggcagcgtttagattcatcatgcagccgtttctgctcttgttcctctcgtcggtcacggcggcgagccccctgacggcgctcgacaagcggcagcaggcgacgttgtgcgagcagtacggctactggtcgggcaacggttacgaggtcaacaacaacaactggggcaaggattcggcctcgggcggccatcagtgcacctacgtcgacagcagcagctccagcggcgtcgcctggcacacgacctggcagtgggaaggaggccagaaccaggtcaagagcttcgccaactgcggcctgcaggtgcccaagggcaggaccatctcgtccatcagcaacctgcagacctccatctcgtggtcctacagcaacaccaacatccgcgccaacgtggcctacgacctcttcaccgcggcagacccgaaccacgcgaccagcagcggcgactacgagctcatgatctggtcagttttttttttcttttttcttttcttctcttttcttttcttttcctttctcctgttttattttcttatccattgcttcgccctctttccttaaccctgctgactctctcttcttgtcaatgatactgtaataggctggcgagattcggcgacgtctaccccatcggctcgtcccagggccacgtcaacgtggccggccaggactgggagctgtggacgggcttcaacggcaacatgcgggtctacagcttcgtagcgcccagcccccgcaacagcttcagcgccaacgtcaaggacttcttcaactatctccagtccaaccagggcttcccggccagcagccaataccttctcagtaaggagacgagatctcgaacagcataccatatatgcgtgcggtacaagtgcactaacccccttttttttcccgttcgcagtcttccaggcgggcaccgagcccttcaccggcggcgagaccacccttaccgtcaacaactactctgcaagggttgcttaaacaggaaggccgaggatggcccccaaggccgttgcgggttcacgagctctcttcttttcaagtgctgtacatacataattagcgtaccaagtcatagctgtttgtcagcttcaaactaagtgctcgcccacaaaagaggggggaggggaaaataacaaattgccgaacgcagtgataagcttctgggagcgttgaaagcagtctacagtaggtggctgtacgaaggaaaagagtgcctta

SEQ ID NO: 25 - protein, 225 aa, Myceliophthora thermophila
MHLSATTGFLALPALALAQLSGSGQTTRYWDCCKPSCAWPGKGPSSPVQACDKNDNPLNDGGSTRSGCDAGGSAYMCSSQSPWAVSDELSYGWAAVKLAGSSESQWCCACYELTFTSGPVAGKKMIVQATNTGGDLGDNHFDLAIPGGGVGIFNACTDQYGAPPNGWGDRYGGIHSKEECESFPEALKPGCNWRFDWFQNADNPSVTFQEVACPSELTSKSGCSR

SEQ ID NO: 26 - protein, 207 aa, Myceliophthora thermophila
QLSGSGQTTRYWDCCKPSCAWPGKGPSSPVQACDKNDNPLNDGGSTRSGCDAGGSAYMCSSQSPWAVSDELSYGWAAVKLAGSSESQWCCACYELTFTSGPVAGKKMIVQATNTGGDLGDNHFDLAIPGGGVGIFNACTDQYGAPPNGWGDRYGGIHSKEECESFPEALKPGCNWRFDWFQNADNPSVTFQEVACPSELTSKSGCSR

SEQ ID NO: 27 - DNA, 675 bp, Artificial
  cDNA encoding MtEG V
atgcatctctccgccaccaccgggttcctcgccctcccggccctggccctggcccagctctcgggcagcggccagacgacccggtactgggactgctgcaagccgagctgcgcctggcccggcaagggcccctcgtctccggtgcaggcctgcgacaagaacgacaacccgctcaacgacggcggctccacccggtccggctgcgacgcgggcggcagcgcctacatgtgctcctcccagagcccctgggccgtcagcgacgagctgtcgtacggctgggcggccgtcaagctcgccggcagctccgagtcgcagtggtgctgcgcctgctacgagctgaccttcaccagcgggccggtcgcgggcaagaagatgattgtgcaggcgaccaacaccggtggcgacctgggcgacaaccactttgacctggccatccccggtggcggtgtcggtattttcaacgcctgcaccgaccagtacggcgctcccccgaacggctggggcgaccgctacggcggcatccattccaaggaagagtgcgaatccttcccggaggccctcaagcccggctgcaactggcgcttcgactggttccaaaacgccgacaacccgtcggtcaccttccaggaggtggcctgcccgtcggagctcacgtccaagagcggctgctcccgt

SEQ ID NO: 28 - DNA, 1448 bp, Myceliophthora thermophila
atatataagtgagagtgttttttgactgccccgggttctggtagttgaagggaagttcgatgctctctgctgtcgtcgctctcgtcgctctcgtcggcatcctccatccgtccgcctttgataacccgctccccgactcagtcaagacgacgcatacttggcaccatgcatctctccgccaccaccgggttcctcgccctcccggccctggccctggcccagctctcgggcagcggccagacgacccggtactgggactgctgcaagccgagctgcgcctggcccggcaagggcccctcgtctccggtgcaggcctgcgacaagaacgacaacccgctcaacgacggcggctccacccggtccggctgcgacgcgggcggcagcgcctacatgtgctcctcccagagcccctgggccgtcagcgacgagctgtcgtacggctgggcggccgtcaagctcgccggcagctccgagtcgcagtggtgctgcgcctgctacgagctgaccttcaccagcgggccggtcgcgggcaagaagatgattgtgcaggcgaccaacaccggtggcgacctgggcgacaaccactttgacctggccgtgagttgcctccccttctccccggaccgctcagattagatgagattagactttgctcgtaaatcggtccaagattcccgttgactgaccaacaaacatcatacgggcagatccccggtggcggtgtcggtattttcaacggtaagctggtgcccccggacccctccccggacccctcccccttttcctccagcgagccgagttgggatcgccgagatcgagaactcacacaacttctctctcgacagcctgcaccgaccagtacggcgctcccccgaacggctggggcgaccgctacggcggcatccattccaaggaagagtgcgaatccttcccggaggccctcaagcccggctgcaactggcgcttcgactggtacgttgctttgacataccggaacccaattcctccaacccccccccttttctcccccaactccgggggtagtcggaatgtcgcgactgaccctatttcaggttccaaaacgccgacaacccgtcggtcaccttccaggaggtggcctgcccgtcggagctcacgtccaagagcggctgctcccgttaagagggaagagagggggctggaaggaccgaaagattcaacctctgctcctgctggggaagctcgggcgcgagtgtgaaactggtgtaaatattgtggcacacacaagctactacagtccgtctcgccgtccggctaactagccttgctgcggatctgtccatcttcggtccgaactgtccgttgctgttttggctcggtgcctcatcttctcccaacctagtcaagaatgaatcgtgagagaggctgagagagataagatcgacttcagaaatccagggttgaaagca

SEQ ID NO: 29 - protein, 381 aa, Myceliophthora thermophila
MRVSSLVAALATGGLVAATPKPKGSSPPGAVDANPFKGKTQFVNPAWAAKLEQTKKAFLARNDTVNAAKTEKVQQTSSFVWVSRIAELSNIDDAIAAARKAQKKTGRRQIVGLVLYNLPDRDCSAGESAGELSSDKNGLEIYKTEFVKPFADKVAAAKDLDFAIVLEPDSLANLVTNLGIEFCANAAPVYREGIAYAISSLQQPNVHLYIDAAHGGWLGWDDNLPLAAKEFAEVVKLAGEGKKIRGFVTNVSNYNPFHAVVRENFTEWSNSWDESHYASSLTPFLEKEGLPARFIVDQGRVALPGARKEWGEWCNVAPAGFGPAPTTRVNNTVVDALVWVKPGGESDGECGLAGAPKAGQWFDEYAQMLVENAHPSVVHKW

SEQ ID NO: 30 - protein, 363 aa, Myceliophthora thermophila
TPKPKGSSPPGAVDANPFKGKTQFVNPAWAAKLEQTKKAFLARNDTVNAAKTEKVQQTSSFVWVSRIAELSNIDDAIAAARKAQKKTGRRQIVGLVLYNLPDRDCSAGESAGELSSDKNGLEIYKTEFVKPFADKVAAAKDLDFAIVLEPDSLANLVTNLGIEFCANAAPVYREGIAYAISSLQQPNVHLYIDAAHGGWLGWDDNLPLAAKEFAEVVKLAGEGKKIRGFVTNVSNYNPFHAVVRENFTEWSNSWDESHYASSLTPFLEKEGLPARFIVDQGRVALPGARKEWGEWCNVAPAGFGPAPTTRVNNTVVDALVWVKPGGESDGECGLAGAPKAGQWFDEYAQMLVENAHPSVVHKW

SEQ ID NO: 31 - DNA, 1143 bp, Artificial
  cDNA encoding MtEG VI
atgcgcgtctctagtttggtcgcggcccttgctaccggtggtcttgtcgccgccacgcctaagcccaaggggtcgtcgccccctggggccgtggacgcgaaccctttcaagggcaagacgcagttcgtcaacccggcatgggcggccaagctggaacagaccaaaaaggcgttcctggccaggaacgacaccgtcaatgccgccaagacggagaaggtccagcagaccagctcgttcgtctgggtctcgaggatcgccgagctctccaacatcgacgacgccatcgcggctgcccgcaaggcgcagaagaagacgggcaggaggcagatcgtcggcctggtgctctacaaccttccggaccgcgactgcagcgcgggcgagagcgcgggcgagctcagcagcgacaagaacgggctcgagatctacaagactgagttcgtcaagcccttcgccgacaaggtggcggccgcaaaggacctcgacttcgccatcgtcctggagcccgactcgctggccaacctggtcaccaacctgggcatcgagttctgcgccaacgccgcccccgtctaccgcgagggcatcgcctatgccatctccagccttcagcagccaaacgtgcacttgtacatcgatgctgcccacggcggctggctcggctgggacgacaacctgccgctggccgccaaggagtttgccgaggtggtcaagcttgccggcgagggcaagaagatccgcggcttcgtcaccaacgtgtccaactacaaccccttccacgccgtcgtgcgcgagaactttaccgagtggagcaactcgtgggacgagtctcactacgcctcctcgctcacaccgttcctcgagaaagaggggctgccggcacgcttcatcgtcgaccagggtcgcgttgccctcccgggagcccgcaaggagtggggtgaatggtgcaacgtggcacccgccggatttggccccgcgcccacgaccagggtcaacaacaccgtcgtcgatgctctcgtctgggtcaagcctggcggcgagagcgacggcgagtgtggcttggctggcgcccccaaggccggccagtggttcgacgagtacgcccagatgctggtcgagaatgcccacccgtctgtcgtccacaagtgg

SEQ ID NO: 32 - DNA, 1658 bp, Myceliophthora thermophila
aggaacggggaggaggagggcttggttagggtcgcgttcgtttggagattgctgagctctgagccttcggtccttggatccctgcggtccccggtctcctctctctctctctctctctctctctctctctcttcttcccacgctcgttcgacagacgcctccccttcttcgctctcctttccctcgcacgtagcacactaatagtgcaccatgcgcgtctctagtttggtcgcggcccttgctaccggtggtcttgtcgccgccacgcctaagcccaaggggtcgtcgccccctggggccgtggacgcgaaccctttcaagggcaagacgcagttcgtcaacccggcatgggcggccaagctggaacagaccaaaaaggcgttcctggccaggaacgacaccgtcaatgccgccaagacggagaaggtccagcagaccagctcgttcgtctgggtctcgaggatcgccgagctctccaacatcgacgacgccatcgcggctgcccgcaaggcgcagaagaagacgggcaggaggcagatcgtcggcctggtgctctacaaccttccggaccgcgactgcagcgcgggcgagagcgcgggcgagctcagcagcgacaagaacgggctcgagatctacaagactgagttcgtcaagcccttcgccgacaaggtggcggccgcaaaggacctcgacttcgccatcgtcctggagcccgactcgctggccaacctggtcaccaacctgggcatcgagttctgcgccaacgccgcccccgtctaccgcgagggcatcgcctatgccatctccagccttcagcagccaaacgtgcacttgtacatcgatgctgcccacggcggctggctcggctgggacgacaacctgccgctggccgccaaggagtttgccgaggtggtcaagcttgccggcgagggcaagaagatccgcggcttcgtcaccaacgtgtccaactacaaccccttccacgccgtcgtgcgcgagaactttaccgagtggagcaactcgtgggacgagtctcactacgcctcctcgctcacaccgttcctcgagaaagaggggctgccggcacgcttcatcgtcgaccagggtcgcgttgccctcccgggagcccgcaaggagtggtgagtttcgaccagattgaccctcgacccatgcgaccgagattgctgacgattgaattgcgtgtcccgtcccccaggggtgaatggtgcaacgtggcacccgccggatttggccccgcgcccacgaccagggtcaacaacaccgtcgtcgatgctctcgtctgggtcaagcctggcggcgagagcgacggcgagtgtggcttggctggcgcccccaaggccggccagtggttcgacgagtacgcccagatgctggtcgagaatgcccacccgtctgtcgtccacaagtggtagataaattttggagtccgagaagggtcccagatagacttttgttttaaaacaaaatgcaaggtgtcgacagatactggcttaacattaaccaagcaccatgaacatgacttgtcaacatattgatacattccgctgctttcccatacgtgctctcaggtctcagggatcaaatggataggtcggtaatgcaaaacgatccattggatatccagaagagagaaaaaa

SEQ ID NO: 33 - protein, 402 aa, Myceliophthora thermophila
RCSSDAPPPAPVGDDLTEPKELTDLFEKAKKAVIDRLHEDEKALRARGEAPRCTADKLIFRREYGSLSKDERLAYVNAVKCLQSKPPRTPASVAPGARSRFDDFVVVHIQQTLDIHYSGIFQAWHRWFVYQYEKALRDECGYTGYQPYWDWPKYASAPQDSPLFNGDPYSLGGNGEYVPHDGPVIVPPEGVSGGNISLPAGVGGGFVRTGPFANMTVNLGPVGGLADTAPGPQGGLGYNPRGLKRDLGGAMNTRYANYTTVLRLLTQPDVDAFRTVSEGVPYTVEIGPHGGIHYTIGGDPGGDLFTSPGDPAFWVHHAQMDRVWATWQALGLLPPADGGDPDPARRYTDLGKGDYAHRTWQNSPPSPFAELSDVIDMGYAAPSTTIGAVMSTTEGELCYFYL

SEQ ID NO: 34 - protein, 424 aa, Myceliophthora thermophila
MKPAALLGAALAAVAFPAGAHARCSSDAPPPAPVGDDLTEPKELTDLFEKAKKAVIDRLHEDEKALRARGEAPRCTADKLIFRREYGSLSKDERLAYVNAVKCLQSKPPRTPASVAPGARSRFDDFVVVHIQQTLDIHYSGIFQAWHRWFVYQYEKALRDECGYTGYQPYWDWPKYASAPQDSPLFNGDPYSLGGNGEYVPHDGPVIVPPEGVSGGNISLPAGVGGGFVRTGPFANMTVNLGPVGGLADTAPGPQGGLGYNPRGLKRDLGGAMNTRYANYTTVLRLLTQPDVDAFRTVSEGVPYTVEIGPHGGIHYTIGGDPGGDLFTSPGDPAFWVHHAQMDRVWATWQALGLLPPADGGDPDPARRYTDLGKGDYAHRTWQNSPPSPFAELSDVIDMGYAAPSTTIGAVMSTTEGELCYFYL

SEQ ID NO: 35 - DNA, 1275 bp, Artificial
  cDNA encoding PPO2
atgaaacctgccgcgctgctcggagcggcccttgcggccgtggccttccccgcgggcgcccatgcccgctgttcttccgatgcgccgcctcccgcccccgttggcgatgacctcaccgagcccaaggagctgacggacctcttcgagaaggccaagaaggccgtcatcgatcgcctccacgaggacgagaaggccctgcgcgcccgcggcgaggcgcctcgctgcaccgccgataagctcatctttcgccgcgaatacgggtctctgtccaaggatgagcgtctcgcgtacgtcaacgcggtcaagtgcctgcagagcaagccgccgcggacgccggcgagcgtggcgcccggggcgcggtcgcggttcgacgactttgtggtggtgcacatccagcagacgctcgacatccactactcgggcatcttccaggcgtggcaccgctggttcgtgtaccagtacgagaaggcgctgcgggacgagtgcggctacacgggctaccagccgtattgggactggcccaagtacgcgagcgcgccccaggactcgcccctgttcaacggcgacccgtacagcctgggcggcaacggcgagtacgtgccccacgacgggcccgtcatcgtgccgcccgagggcgtcagcggcggcaacatctcgctcccggccggcgtcggcggcggcttcgtcaggacggggcccttcgccaacatgacggtcaacctgggccccgtcggcggcctggccgacaccgccccgggcccccagggcggcctcggctacaacccgcgcgggctcaagcgcgacctgggcggggccatgaacacgcgctacgccaactacaccaccgtcctgcgcctgctgacccagcccgacgtcgacgccttccgcaccgtctccgagggcgtcccctacacggtcgagatcggcccccacggcggcatccactacaccatcggcggcgacccgggcggcgacctcttcacctcccccggcgacccggccttctgggtccaccacgcccagatggaccgcgtctgggccacctggcaggccctcggcctcctcccccccgccgacggcggcgaccccgacccggcccgccgctacaccgacctcggcaagggcgactacgcccaccgcacctggcagaactccccgccctccccctttgccgagctgtccgacgtcatcgatatgggctacgccgccccctccaccaccatcggcgccgtcatgtccaccaccgaaggcgagctgtgctacttttatctctga

SEQ ID NO: 36 - DNA, 1490 bp, Myceliophthora thermophila
atgaaacctgccgcgctgctcggagcggcccttgcggccgtggccttccccgcgggcgcccatgcccgctgttcttccgatgcgccgcctcccgcccccgttggcgatgacctcaccgagcccaaggagctgacggacctcttcgagaaggccaagaaggccgtcatcgatcgcctccacgaggacgagaaggccctgcgcgcccgcggcgaggcgcctcgctgcaccgccgataagctcatctttcgccgcgaatagtacgttcatgatgcctcttcttagtccctttataatacacaaacaccgaatattcccacgctttaattcgtgtttttttttttggcgttcttctcttcaatcatgcccatgttttatacaaaggcatcatgaaccgtgtcccccccttgacgtgagagtgtttgctgaccggccgactttgttccatcgtcgccgtcgtcgtcgtcgtccacagcgggtctctgtccaaggatgagcgtctcgcgtacgtcaacgcggtcaagtgcctgcagagcaagccgccgcggacgccggcgagcgtggcgcccggggcgcggtcgcggttcgacgactttgtggtggtgcacatccagcagacgctcgacatccactactcgggcatcttccaggcgtggcaccgctggttcgtgtaccagtacgagaaggcgctgcgggacgagtgcggctacacgggctaccagccgtattgggactggcccaagtacgcgagcgcgccccaggactcgcccctgttcaacggcgacccgtacagcctgggcggcaacggcgagtacgtgccccacgacgggcccgtcatcgtgccgcccgagggcgtcagcggcggcaacatctcgctcccggccggcgtcggcggcggcttcgtcaggacggggcccttcgccaacatgacggtcaacctgggccccgtcggcggcctggccgacaccgccccgggcccccagggcggcctcggctacaacccgcgcgggctcaagcgcgacctgggcggggccatgaacacgcgctacgccaactacaccaccgtcctgcgcctgctgacccagcccgacgtcgacgccttccgcaccgtctccgagggcgtcccctacacggtcgagatcggcccccacggcggcatccactacaccatcggcggcgacccgggcggcgacctcttcacctcccccggcgacccggccttctgggtccaccacgcccagatggaccgcgtctgggccacctggcaggccctcggcctcctcccccccgccgacggcggcgaccccgacccggcccgccgctacaccgacctcggcaagggcgactacgcccaccgcacctggcagaactccccgccctccccctttgccgagctgtccgacgtcatcgatatgggctacgccgccccctccaccaccatcggcgccgtcatgtccaccaccgaaggcgagctgtgctacttttatctctga

SEQ ID NO: 37 - protein, 352 aa, Myceliophthora thermophila
DAVLDLQEKGRPAIDAQLAKSTTCTKEKLQVRREWGDISAEEKKAYIAAMLCIMEKPSRLDPNQFPGAKSRYDDFVVVHMNQTLSIHGTGNFLSWHRYYTWVFERALQEECGYNGTQPYWDWGRWADDPENSPIFDGSDTSLSGNGEYIEHRASGFIPAGNGGGCVKSGPFKDMVVHLGPVAPAIDPAPPANPRRDGYGDNPRCLRRDISNQLSSKYARTQDIVDLITTSADIGTFQTVMQGAGGFGVGPGGMGVHAAGHFTIAGDPGGDFYTSPNDPAFWVHHGMIDRTWTIWQSQDLDNRLQVIAGGSSMMGGPPQTLDDPVDLGVVADKVYPIRDLVSVVDGPFCYVYE

SEQ ID NO: 38 - protein, 375 aa, Myceliophthora thermophila
MARLTLAGLLAVPWLLLPGLVSGDAVLDLQEKGRPAIDAQLAKSTTCTKEKLQVRREWGDISAEEKKAYIAAMLCIMEKPSRLDPNQFPGAKSRYDDFVVVHMNQTLSIHGTGNFLSWHRYYTWVFERALQEECGYNGTQPYWDWGRWADDPENSPIFDGSDTSLSGNGEYIEHRASGFIPAGNGGGCVKSGPFKDMVVHLGPVAPAIDPAPPANPRRDGYGDNPRCLRRDISNQLSSKYARTQDIVDLITTSADIGTFQTVMQGAGGFGVGPGGMGVHAAGHFTIAGDPGGDFYTSPNDPAFWVHHGMIDRTWTIWQSQDLDNRLQVIAGGSSMMGGPPQTLDDPVDLGVVADKVYPIRDLVSVVDGPFCYVYE

SEQ ID NO: 39 - DNA, 1128 bp, Artificial
  cDNA encoding PPO4
atggcacgtctcaccctcgcggggctgcttgcagtcccctggctcctgctcccgggcctcgtgtccggcgacgcagtcttggatctccaggaaaagggtcgtcccgccatcgatgctcaactggccaagtcgacgacatgcaccaaagagaaactccaggtccgccgcgagtggggcgacatctcggccgaagagaagaaggcctacatcgccgccatgctgtgcatcatggagaagccgtccaggttggacccgaaccagtttcccggcgccaagtcacggtacgacgacttcgtcgtcgttcacatgaaccagactctgtccatccacggcacgggcaacttcctctcgtggcatcgctactacacctgggtgtttgagcgcgccctccaggaggagtgcgggtataacgggacccagccctactgggactgggggcgctgggccgacgacccggagaactcgcccatctttgacggcagcgacacctctctcagcggcaacggcgagtacattgagcacagggcctcgggcttcatacccgccggcaacggaggtggctgcgtcaagtcgggtcccttcaaagacatggtcgtccacctgggccccgtcgctcccgccatcgacccggcgccgccggcgaacccgcggcgcgacgggtacggcgacaacccgcggtgcctgcggcgtgacatctcgaaccagctgtcgagcaagtacgcgcggacgcaggacattgtcgacctgatcacgacgtcggccgacattggcaccttccagacggtcatgcagggcgccggcggcttcggcgtcggccccggcggcatgggcgtgcacgccgccggccacttcaccatcgccggcgacccgggcggcgacttctacacctcccccaacgacccggccttctgggtccaccacggcatgatcgaccgcacctggaccatctggcagagccaggacctcgacaaccgcctccaggtcatcgccggcggttccagcatgatgggcggccctccccagaccctcgacgaccccgttgacctcggcgtcgtcgccgacaaggtctatcccatcagggacctggtcagcgtcgtcgacgggcccttctgctacgtctatgagtga

SEQ ID NO: 40 - DNA, 1468 bp, Myceliophthora thermophila
atggcacgtctcaccctcgcggggctgcttgcagtcccctggctcctgctcccgggcctcgtgtccggcgacgcagtcttggatctccaggaaaagggtcgtcccgccatcgatgctcaactggccaagtcgacgacatgcaccaaagagaaactccaggtccgccgcgagtggtgagtctgccgccgccgtcgtcctccttaagtatccggttaagccgtgatgtccacgcaaactgacagagagggggggggggttgaaaccaggggcgacatctcggccgaagagaagaaggcctacatcgccgccatgctgtgcatcatggagaagccgtccaggttggacccgaaccagtttcccggcgccaagtcacggtacgacgacttcgtcgtcgttcacatgaaccagactctgtccatccacggcacggtaattaattgccttccccgagaggttgccctcacctcagtgccatggcatcgctgactgactcttcttcttcttcttcttcttcttcttcttcacagggcaacttcctctcgtggcatcgctactacacctgggtgtttgagcgcgccctccaggaggagtgcgggtataacgggacccagcccgtaagactctcccccccccccccggggccccgccaaataacccgtattatgcccgctgaccacttttgcacagtactgggactgggggcgctgggccgacgacccggagaactcgcccatctttgacggcagcgacacctctctcagcggcaacggcgagtacattgagcacagggcctcgggcttcatacccgccggcaacggaggtggctgcgtcaagtcgggtcccttcaaagagtgagtctcatcatcacttattagttgtctcttttatctctctccaaccctttcctggctaaccccttcgccccagcatggtcgtccacctgggccccgtcgctcccgccatcgacccggcgccgccggcgaacccgcggcgcgacgggtacggcgacaacccgcggtgcctgcggcgtgacatctcgaaccagctgtcgagcaagtacgcgcggacgcaggacattgtcgacctgatcacgacgtcggccgacattggcaccttccagacggtcatgcagggcgccggcggcttcggcgtcggccccggcggcatgggcgtgcacgccgccggccacttcaccatcgccggcgacccgggcggcgacttctacacctcccccaacgacccggccttctgggtccaccacggcatgatcgaccgcacctggaccatctggcagagccaggacctcgacaaccgcctccaggtcatcgccggcggttccagcatgatgggcggccctccccagaccctcgacgaccccgttgacctcggcgtcgtcgccgacaaggtctatcccatcagggacctggtcagcgtcgtcgacgggcccttctgctacgtctatgagtga

SEQ ID NO: 41 - protein, 510 aa, Myceliophthora thermophila
AVKTVVITATTTVCPLSTRVCHGSSMISQSTNAPCSESTGITGSQSTAVTIPQSTDIISSQFSQFTDTTSSLSTGVACSSSPCPSNSQSTGTTSFESTGTTISQPTAVTNSSSTCLTSSQSTDAPSSESTGITISQSTGVTSSQSMSTPTSKSTAVPSSQSARVSTTSSSAKPSSTTETRPEPTLPNRAYPADIVDKLRDSGVDKLKEYLKSNPSNTGCTLENASIRREWSDLSVAEREEYIAAVKCLQSASPKSSKQQVPGARSRFDDFVATHINQTDSIHYTANFLSWHRYYVWAYEKALREECGYTGFQPYWNWDRYAADPANSPLFSGNSSSLSGNSVNGGCVTTGPFADMKVNLGPGASLAYNPRCLKRNISKSYAAMTTADKTYALITGSADIARFQDTMQAVPGVHAGGHFTIGGDPGGDVYSSPGDPAFWLHHAMIDRVWWIWQTHGLPGRLSEVAGSVAGSSRPGSGKDLVNLGVNGPAVAIENLLNTVGGLDGKMCYIYL

SEQ ID NO: 42 - protein, 529 aa, Myceliophthora thermophila
MQLSLLSTGALLLGVGTSHAVKTVVITATTTVCPLSTRVCHGSSMISQSTNAPCSESTGITGSQSTAVTIPQSTDIISSQFSQFTDTTSSLSTGVACSSSPCPSNSQSTGTTSFESTGTTISQPTAVTNSSSTCLTSSQSTDAPSSESTGITISQSTGVTSSQSMSTPTSKSTAVPSSQSARVSTTSSSAKPSSTTETRPEPTLPNRAYPADIVDKLRDSGVDKLKEYLKSNPSNTGCTLENASIRREWSDLSVAEREEYIAAVKCLQSASPKSSKQQVPGARSRFDDFVATHINQTDSIHYTANFLSWHRYYVWAYEKALREECGYTGFQPYWNWDRYAADPANSPLFSGNSSSLSGNSVNGGCVTTGPFADMKVNLGPGASLAYNPRCLKRNISKSYAAMTTADKTYALITGSADIARFQDTMQAVPGVHAGGHFTIGGDPGGDVYSSPGDPAFWLHHAMIDRVWWIWQTHGLPGRLSEVAGSVAGSSRPGSGKDLVNLGVNGPAVAIENLLNTVGGLDGKMCYIYL

SEQ ID NO: 43 - DNA, 1590 bp, Artificial
  cDNA encoding PPO6
atgcagctttctttgttgagtaccggagcgctgctcctcggggttgggaccagtcatgcggtaaagacggtcgtgattaccgctaccacgaccgtctgccccctttctacacgcgtctgtcatgggtcgtcaatgatctcgcagtctacgaatgcgccgtgctcggagtctacgggtataacgggttcgcaatctacggctgtaacgatcccgcagtctacggacattattagttcgcaattttcgcaattcacggacaccacgagttcgctgtctacgggtgtggcgtgctcgtcgtctccgtgtccctcaaactcgcagtctacaggcacaacgagtttcgagtctacggggacgactatctctcagcctacggctgtaacgaactcgtcgtctacctgtctcacaagctcgcagtctaccgatgcaccgagctcggagtctacgggcattacaatttctcaatctacgggtgtaacgagctcgcagtctatgagtacaccgacttccaagtctacggctgtaccgagctcccaatctgctcgggtctcgacgacatcatcttcggccaagccctcgtccactacggaaacacgtcctgaacccaccctgcccaatcgagcttatcccgccgacattgtcgacaagctccgagactcaggtgtggacaagctcaaggagtacctcaagagcaatccctcgaatacaggctgcacgctcgagaacgcgtctatccgccgcgaatggtccgacctctccgtcgccgagcgagaggagtacattgccgccgtcaagtgcctgcagtcggcgagccccaagtcttccaaacagcaagtcccgggcgcgcgtagccgcttcgacgactttgtggccacgcacattaaccagaccgactcgatccattacacggcgaacttcctctcgtggcaccgatactacgtgtgggcatacgagaaggccctccgcgaggagtgcggctacaccggcttccagccgtactggaactgggaccggtacgcagccgacccggccaactcgcccctgttcagcggcaactcgtccagcctgagcggaaactcggtcaacggcggttgtgtcaccacgggccctttcgccgacatgaaagtcaacctcggccccggggcctctctcgcctacaacccgcgctgcctgaagcgcaacatcagcaagagctacgcggccatgaccacggccgacaagacgtatgcgctcatcacgggtagcgccgacattgcccgcttccaggacaccatgcaggccgtccctggcgtgcatgccggcggccactttaccatcggcggcgaccctggcggcgacgtctactcgtcgccgggcgacccggccttctggctgcaccacgccatgatcgaccgcgtctggtggatctggcagacgcacggcctgcccggccggctgtccgaggtggccggttccgtcgccggctccagccgcccgggctccggcaaggacctcgtcaacctgggcgtcaacggccccgccgtcgccatcgagaacctgctcaacaccgtgggcggcctggacggcaagatgtgctacatctacctctga

SEQ ID NO: 44 - DNA, 2051 bp, Myceliophthora thermophila
atgcagctttctttgttgagtaccggagcgctgctcctcggggttgggaccagtcatgcggtaaagacggtcgtgattaccgctaccacgaccgtctgccccctttctacacgcgtctgtcatgggtcgtcaatgatctcgcagtctacgaatgcgccgtgctcggagtctacgggtataacgggttcgcaatctacggctgtaacgatcccgcagtctacggacattattagttcgcaattttcgcaattcacggacaccacgagttcgctgtctacgggtgtggcgtgctcgtcgtctccgtgtccctcaaactcgcagtctacaggcacaacgagtttcgagtctacggggacgactatctctcagcctacggctgtaacgaactcgtcgtctacctgtctcacaagctcgcagtctaccgatgcaccgagctcggagtctacgggcattacaatttctcaatctacgggtgtaacgagctcgcagtctatgagtacaccgacttccaagtctacggctgtaccgagctcccaatctgctcgcgtctcgacgacatcatcttcggccaagccctcgtccactacggaaacacgtcctgaacccaccctgcccaatcgagcttatcccgccgacattgtcgacaagctccgagactcaggtgtggacaagctcaaggagtacctcaagagcaatccctcgaatacaggctgcacgctcgagaacgcgtctatccgccgcgaatggtaagccacccaccccccctgcttctcgaacagtaattacgttccgtcgagacgagagaaaccaaggctaacaagaggcacctccggagcaaacaggtccgacctctccgtcgccgagcgagaggagtacattgccgccgtcaagtgcctgcagtcggcgagccccaagtcttccaaacagcaagtcccgggcgcgcgtagccgcttcgacgactttgtggccacgcacattaaccagaccgactcgatccattacacggtacgtattcggccttgcgtacttcccatcctttctctacgtatctatctcccttgtccagttagcgagcccgtcatatgtttgagactgacttcgcatgtccctcgattcaaggcgaacttcctctcgtggcaccgatactacgtgtgggcatacgagaaggccctccgcgaggagtgcggctacaccggcttccagccgtactggaactgggaccggtacgcagccgacccggccaactcgcccctgttcagcggcaactcgtccagcctgagcggaaactcggtcaacggcggttgtgtcaccacgggccctttcgccgagtaagttctttgctcctcttccccccataaggggtggccagataattattcagacattgctaaccgggcctctcccttcgcttcagcatgaaagtcaacctcggccccggggcctctctcgcctacaacccgcgctgcctgaagcgcaacatcagcaagagctacgcggccatgaccacggccgacaagacgtatgcgctcatcacgggtagcgccgacattgcccgcttccaggacaccatgcaggccgtccctggcgtgcatgccggcggccactttaccatcggcggcgaccctggcggcgtaagttggatttttgattctccgacccttccccccccccccccccaaaaaaaaaaaaaaaaaccaaccctcctttcttttctcagaaagagaagagcccttttatttttttccctctgtcgcctggcccagctgacacgagttctctcttgggaacaacgacaggacgtctactcgtcgccgggcgacccggccttctggctgcaccacgccatgatcgaccgcgtctggtggatctggcagacgcacggcctgcccggccggctgtccgaggtggccggttccgtcgccggctccagccgcccgggctccggcaaggacctcgtcaacctgggcgtcaacggccccgccgtcgccatcgagaacctgctcaacaccgtgggcggcctggacggcaagatgtgctacatctacctctga

SEQ ID NO: 45 - protein, 402 aa, Myceliophthora thermophila
NPMHKRWKPNAPRNPDFPLDEVDKLEESIMPNVEAWMSKRPAKYSNCTLENAGIRREWSDLSVDERKEYISAVLCLQSKPSKIPEGEVPGALSRFDDFVATHMTMAGQLHSPTNLFAAHRYFIHAYEKALREECGYKGYQPYMNYDRYADDPLNTPLFDGSDSSMSGNGGPYNYSGIPQPFPKPYNLIPPAGGGGCVTEGPFRDMVVSLGPKGGLFHDIPRNPRADGLGSNPRCLRRDVNKYSARGARANYTYSAIMDNPDIDSFYNRYMGMPQLKGDPYPWGLHSSGHYLIGGDPGGDFYCSPGDPLFYFHHGMLDRVWWIWQMQDPENRVQAVPGTNTMPMPPLMAANLESEAAATTTHKRAVNVSDLVVDLGWTAPPVKLVDLNEQLGGLGGELCYIYV

SEQ ID NO: 46 - protein, 420 aa, Myceliophthora thermophila
MRGIQSLVSLSLLAGAWANPMHKRWKPNAPRNPDFPLDEVDKLEESIMPNVEAWMSKRPAKYSNCTLENAGIRREWSDLSVDERKEYISAVLCLQSKPSKIPEGEVPGALSRFDDFVATHMTMAGQLHSPTNLFAAHRYFIHAYEKALREECGYKGYQPYMNYDRYADDPLNTPLFDGSDSSMSGNGGPYNYSGIPQPFPKPYNLIPPAGGGGCVTEGPFRDMVVSLGPKGGLFHDIPRNPRADGLGSNPRCLRRDVNKYSARGARANYTYSAIMDNPDIDSFYNRYMGMPQLKGDPYPWGLHSSGHYLIGGDPGGDFYCSPGDPLFYFHHGMLDRVWWIWQMQDPENRVQAVPGTNTMPMPPLMAANLESEAAATTTHKRAVNVSDLVVDLGWTAPPVKLVDLNEQLGGLGGELCYIYV

SEQ ID NO: 47 - DNA, 1263 bp, Artificial
  cDNA encoding PPO7
atgcgagggattcagagtctcgtgagcctgtccttgctcgccggagcttgggcgaacccgatgcacaagagatggaagcccaacgcgccccggaacccggacttcccccttgacgaagtcgacaagctcgaggagtccatcatgcccaatgttgaggcatggatgtccaagcgcccggccaaatacagcaactgcactctcgagaatgcgggcatccggagggaatggagcgacctctcggtggacgagcgcaaggagtacatcagcgccgtcctctgcctccagtccaagccttccaagattcccgagggcgaggtccctggggccctgagccgcttcgacgactttgtggcgacgcacatgaccatggcaggccagctgcacagcccaaccaacctatttgccgcccaccggtacttcatccacgcctacgagaaggcactgcgtgaggagtgcggctacaaggggtatcagccgtacatgaactacgaccgctatgctgacgaccccctcaacacacccttgtttgacggcagtgacagcagcatgagtggtaacggcggtccgtacaactactccggcatcccccagcccttcccaaagccgtataacttgattcctccggctggtggtggcggttgtgtcaccgagggccccttcagagacatggtagtctcgctgggccccaagggcggcctcttccacgacataccgcgcaacccgcgggcggacggactaggctccaacccgcggtgcctccgccgcgacgtcaacaagtattcggcccgaggcgcccgcgccaactacacctacagcgccatcatggacaacccggatatcgacagcttctacaaccgctacatgggcatgccgcagctcaagggggacccttacccgtggggtctccatagctccggccactacctcatcggtggcgacccgggcggcgacttctactgctctcccggcgacccgctcttctacttccaccacggcatgctcgaccgggtctggtggatctggcagatgcaggacccggagaaccgcgtccaagccgtcccggggaccaacacgatgccgatgccgcccttgatggccgccaacttggagagcgaggcggccgcgacgacgacgcacaagcgggcggtcaacgtcagcgacctggttgtggacctgggctggacggcgcccccggtcaagttggtcgacctgaacgagcagctgggcggtctgggcggcgagctgtgctacatctacgtgtga

SEQ ID NO: 48 - DNA, 1979 bp, Myceliophthora thermophila
atgcgagggattcagagtctcgtgagcctgtccttgctcgccggagcttgggcgaacccgatgcacaagagatggaagcccaacgcgtaggcaatattgtcacgcccaaagaccgtctcgtctctgttcccaactgacatgccgccaggccccggaacccggacttcccccttgacgaagtcgacaagctcgaggagtccatcatgcccaatgttgaggcatggatgtccaagcgcccggccaaatacagcaactgcactctcgagaatgcgggcatccggagggaatggtacgatcagaggccatgccataccggctgactgactgactgactgactgcctgactgattggcctgactgactaataaacgcccaacaggagcgacctctcggtggacgagcgcaaggagtacatcagcgccgtcctctgcctccagtccaagccttccaagattcccgagggcgaggtccctggggccctgagccgcttcgacgactttgtggcgacgcacatgaccatggcaggccagctgcacagcccaaccaacctatttgccgcccaccggtacttcatccacgcctacgagaaggcactgcgtgaggagtgcggctacaaggggtatcagccggtaagagtgacatgaccctttgtttcccctaccttggaaaataagaaacaaaaagttttaaaaggttgttatgttctgttttgtcccactggctgaccctctttctctcgtttccttttaaagtacatgaactacgaccgctatgctgacgaccccctcaacacacccttgtttgacggcagtgacagcagcatgagtggtaacggcggtccgtacaactactccggcatcccccagcccttcccaaagccgtataacttgattcctccggctggtggtggcggttgtgtcaccgagggccccttcagagagtgagttcaagctgacaatctcctcctcctcttgctcctcccttttctcccctcccttttctcccctcccttttctcccctcccttttctcccctcccttttcttccccccctccccccttcttctctcttaattatccccctggcctcttccgcccgggtttgtttcctcggttacatcgttacatcgttacataaaactgacagagaggatgcgcacaacaacagcatggtagtctcgctgggccccaagggcggcctcttccacgacataccgcgcaacccgcgggcggacggactaggctccaacccgcggtgcctccgccgcgacgtcaacaagtattcggcccgaggcgcccgcgccaactacacctacagcgccatcatggacaacccggatatcgacagcttctacaaccgctacatgggcatgccgcagctcaagggggacccttacccgtggggtctccatagctccggccactacctcatcggtggcgacccgggcggcgtaagtagccttttcttgattttttttttgttcttgatttttgttatttattcccccccccttttttttttgtcctgtgattgttttgaagtgtccattaattacctagtcaactgttcaaattaattattatctgtccctcacgcagcactcccagattgccaaccaattctgctaaccatggttctcacctcccctccccccttgggttcaggacttctactgctctcccggcgacccgctcttctacttccaccacggcatgctcgaccgggtctggtggatctggcagatgcaggacccggagaaccgcgtccaagccgtcccggggaccaacacgatgccgatgccgcccttgatggccgccaacttggagagcgaggcggccgcgacgacgacgcacaagcgggcggtcaacgtcagcgacctggttgtggacctgggctggacggcgcccccggtcaagttggtcgacctgaacgagcagctgggcggtctgggcggcgagctgtgctacatctacgtgtga

SEQ ID NO: 49 - protein, 629 aa, Myceliophthora thermophila
QYSGYDYGFDVKKRVKRQLGQRSAMVVQDKTGSEIQVRQEIRQLEQDHDLWTLYILGLSMLQYTDQESPVSYYGLAGIHGMPHQTWGGMGPVTGNENTGYCTHSSVLFPTWHRAYMALYEMIATFWPDSERQRYESAARRFRLPYWDWAASPPPGQSVLPESIGGSPFIDVNGPNGLQRIANPLFSYQFNPLDQKAFEFPPSQWNIWTRTLRSPSSGGPDAQSNNTLVALNLDRSRASIAQRLYDLFSHNDNYTLFSNNAVGGQAESVESLHDTIHSLVGGVGPSQSVPQPGHMTYIQWSAFDPVFFLHHCMVDRIFALWQAIHPNTWVPSSQALLDSYTIRRGQSIDSGTALTPFFSNDNGTFWTSDGVRDHTRFGYTYAELLRGPVTGSSNNTLLAASQIRIVKQAVNRMYGSFSPAFFFLEELRIQGGAAVVGAGFRNNHRKTAPLHGLLESKIFVAGSNGNRYYEWKVDVCVGRNGSQGILGGDGIDGSSISFFLGDLSSLNNLLNDTHVGTMGVFTSARLSQHEPAGGHDVPISGSVPLTAALVKKICEGELAGLASGHVVPYLKRNLKMVMRGTQREVVAGTDDKKICETLLSLRIVSSVVEAPWSEDELPKWGEERVEFDVC

SEQ ID NO: 50 - protein, 651 aa, Myceliophthora thermophila
MARCQMTLLFPIVCLVSALVSAQYSGYDYGFDVKKRVKRQLGQRSAMVVQDKTGSEIQVRQEIRQLEQDHDLWTLYILGLSMLQYTDQESPVSYYGLAGIHGMPHQTWGGMGPVTGNENTGYCTHSSVLFPTWHRAYMALYEMIATFWPDSERQRYESAARRFRLPYWDWAASPPPGQSVLPESIGGSPFIDVNGPNGLQRIANPLFSYQFNPLDQKAFEFPPSQWNIWTRTLRSPSSGGPDAQSNNTLVALNLDRSRASIAQRLYDLFSHNDNYTLFSNNAVGGQAESVESLHDTIHSLVGGVGPSQSVPQPGHMTYIQWSAFDPVFFLHHCMVDRIFALWQAIHPNTWVPSSQALLDSYTIRRGQSIDSGTALTPFFSNDNGTFWTSDGVRDHTRFGYTYAELLRGPVTGSSNNTLLAASQIRIVKQAVNRMYGSFSPAFFFLEELRIQGGAAVVGAGFRNNHRKTAPLHGLLESKIFVAGSNGNRYYEWKVDVCVGRNGSQGILGGDGIDGSSISFFLGDLSSLNNLLNDTHVGTMGVFTSARLSQHEPAGGHDVPISGSVPLTAALVKKICEGELAGLASGHVVPYLKRNLKMVMRGTQREVVAGTDDKKICETLLSLRIVSSVVEAPWSEDELPKWGEERVEFDVC

SEQ ID NO: 51 - DNA, 1956 bp, Artificial
  cDNA encoding PPO1
atggcgcgctgtcaaatgactctcctcttcccgatcgtctgcctcgtcagcgctctcgtttcagcccaatattcaggttatgactatggatttgacgtcaagaagcgggtcaagcgccagctcggccaacgttcggccatggtcgtgcaggacaagaccggcagcgagatccaggtgcgccaggaaattcgtcaacttgagcaggaccatgacctctggaccctgtacatcctaggcttgagcatgctgcagtataccgaccaggaatctcccgtgtcttattatggactcgcggggatacacggaatgcctcaccagacttggggcggcatgggaccagtcaccggaaacgagaataccgggtattgtacccattcgtccgtcctgttcccgacatggcaccgggcatacatggctctatacgagatgatcgccacgttctggcccgacagcgagagacagcgctacgagagtgccgcccgcaggttccgcctcccttactgggactgggcggcaagccctcctccgggacaaagcgttctccctgagagcatcggaggaagtccatttattgacgtcaacggaccgaatggcttgcagcggatcgcgaaccccttattcagctaccagttcaaccctctcgatcagaaggcttttgagttccccccctcacagtggaacatctggactcgaactcttagaagtccatcaagtggcggccctgacgcgcagtccaacaacacgctcgtcgccctgaacctcgatcggagccgcgcctccatcgctcagcgcctctatgatttattttcgcacaacgataactacactctgttcagcaacaatgccgtggggggacaggctgaatcggtagaatcgctgcacgacaccatccatagcttagttggcggcgtcgggcctagtcagtcagtacctcagcctgggcacatgacgtacatccagtggtcggccttcgatcccgtcttcttcctgcatcactgcatggttgaccggattttcgccttgtggcaggcgattcaccccaacacttgggttccctcgtctcaagctctgctggactcgtacaccattcggaggggccagtccatagactctggtacagcactcactcccttcttttccaacgacaacggcacattctggacttctgacggcgtgcgcgaccacacaaggttcggctatacgtacgccgagttgctgcgcggtcccgtcaccggctcttcaaacaataccctcttagccgcatctcaaatacgcatagtaaaacaggccgtaaaccgcatgtatggctctttcagcccggccttcttctttctcgaagagcttcgcattcaaggcggcgctgccgtggtaggcgccggcttccgaaacaaccacaggaaaacggcgccgctccatggtctactggaatccaaaatctttgttgcggggagcaacggcaaccgctactacgagtggaaggtagatgtgtgcgtagggcgcaacggcagccaaggtattctcggcggcgacggaattgacgggtcttcaatcagtttcttccttggcgacctttcttctcttaacaatttgttgaatgacacccatgttgggaccatgggcgtgtttacctccgctcgcttgtcgcaacatgaaccggctggtgggcatgatgtgcctatctcagggagcgtgcctctgacggccgcgctggtgaagaagatctgcgaaggcgagctcgcggggttggcttcaggacatgtggtgccgtacttgaagagaaacctgaagatggtgatgcgagggacccagagagaggtggtggcgggcacagacgataagaagatttgtgaaacactcttgagtcttcggatcgttagctcggtggttgaggcgccgtggagcgaggatgaattgccgaaatggggagaggaaagagtagagtttgatgtgtgctga

SEQ ID NO: 52 - DNA, 2160 bp, Myceliophthora thermophila
atggcgcgctgtcaaatgactctcctcttcccgatcgtctgcctcgtcagcgctctcgtttcagcccaatattcaggttatgactatggatttgacgtcaagaagcgggtcaagcgccagctcggccaacgttcggccatggtcgtgcaggacaagaccggcagcgagatccaggtgcgccaggaaattcgtcaacttgagcaggaccatgacctctggaccctgtacatcctaggcttgagcatgctgcagtataccgaccaggaatctcccgtgtcttattatggactcgcgggtttgtgttgccttgttcgaatgtgtttcggccagtgatgctaacgagatgacggtctaacccagggatacacggaatgcctcaccagacttggggcggcatgggaccagtcaccggaaacgagaataccgggtattgtacccattcgtccgtcctgttcccgacatggcaccgggcatacatggctctatacgaggtatgacagccttcttgtcatagggtgagaaacgcggttattaggttcccgtatcgtatctgacttcagctcagcaagttttgcacgatctaatccagatgatcgccacgttctggcccgacagcgagagacagcgctacgagagtgccgcccgcaggttccgcctcccttactgggactgggcggcaagccctcctccgggacaaagcgttctccctgagagcatcggaggaagtccatttattgacgtcaacggaccgaatggcttgcagcggatcgcgaaccccttattcagctaccagttcaaccctctcgatcagaaggcttttgagttccccccctgtaagtttgggtacattcaaacttgacttgatctgacgaagcacagtggaacatctggactcgaactcttagaagtccatcaagtggcggccctgacgcgcagtccaacaacacgctcgtcgccctgaacctcgatcggagccgcgcctccatcgctcagcgcctctatgatttattttcgcacaacgataactacactctgttcagcaacaatgccgtggggggacaggctgaatcggtagaatcgctgcacgacaccatccatagcttagttggcggcgtcgggcctagtcagtcagtacctcagcctgggcacatgacgtacatccagtggtcggccttcgatcccgtcttcttcctgcatcactgcatggttgaccggattttcgccttgtggcaggcgattcaccccaacacttgggttccctcgtctcaagctctgctggactcgtacaccattcggaggggccagtccatagactctggtacagcactcactcccttcttttccaacgacaacggcacattctggacttctgacggcgtgcgcgaccacacaaggttcggctatacgtacgccgagttgctgcgcggtcccgtcaccggctcttcaaacaataccctcttagccgcatctcaaatacgcatagtaaaacaggccgtaaaccgcatgtatggctctttcagcccggccttcttctttctcgaagagcttcgcattcaaggcggcgctgccgtggtaggcgccggcttccgaaacaaccacaggaaaacggcgccgctccatggtctactggaatccaaaatctttgttgcggggagcaacggcaaccgctactacgagtggaaggtagatgtgtgcgtagggcgcaacggcagccaaggtattctcggcggcgacggaattgacgggtcttcaatcagtttcttccttggcgacctttcttctcttaacaatttgttgaatgacacccatgttgggaccatgggcgtgtttacctccgctcgcttgtcgcaacatgaaccggctggtgggcatgatgtgcctatctcagggagcgtgcctctgacggccgcgctggtgaagaagatctgcgaaggcgagctcgcggggttggcttcaggacatgtggtgccgtacttgaagagaaacctgaagatggtgatgcgagggacccagagagaggtggtggcgggcacagacgataagaagatttgtgaaacactcttgagtcttcggatcgttagctcggtggttgaggcgccgtggagcgaggatgaattgccgaaatggggagaggaaagagtagagtttgatgtgtgctga

SEQ ID NO: 53 - protein, 368 aa, Myceliophthora thermophila
APLGNVDAEAAASQRILELQAQYQRNVLDAISNRTSGCTAQNIQRRQEWSTLSLADRAAFISAIHCLNALPARTPQSTAPGARALYDDFIVAHILQTPFVHASGLFLPFHRHLLHLFAGALRDRCDYAGPLPYWDWTASYADPRAAAVFDGGPHSLGGNGAFVPGRNGTVISVPGGAHVVIPPATGGGCVTTGPFRQGRFEVRLGPVAFEPRGPHGGLGYNPRCLRRDLSPHFSLGTRPGAVVALLDGCAGDLGCLVRDMDAPGGVPGGVHASGHWQVGPDALDVFASPSDPVFWLHHAQVDRVWTIWQGLDLEGRTYQVWGTSTAANDPPSDDVTLETAMDFGILGEAKTVGEVASTVDGEYCYMYE

SEQ ID NO: 54 - protein, 390 aa, Myceliophthora thermophila
MKLSTLPAALLSLILLAHQAAAAPLGNVDAEAAASQRILELQAQYQRNVLDAISNRTSGCTAQNIQRRQEWSTLSLADRAAFISAIHCLNALPARTPQSTAPGARALYDDFIVAHILQTPFVHASGLFLPFHRHLLHLFAGALRDRCDYAGPLPYWDWTASYADPRAAAVFDGGPHSLGGNGAFVPGRNGTVISVPGGAHVVIPPATGGGCVTTGPFRQGRFEVRLGPVAFEPRGPHGGLGYNPRCLRRDLSPHFSLGTRPGAVVALLDGCAGDLGCLVRDMDAPGGVPGGVHASGHWQVGPDALDVFASPSDPVFWLHHAQVDRVWTIWQGLDLEGRTYQVWGTSTAANDPPSDDVTLETAMDFGILGEAKTVGEVASTVDGEYCYMYE

SEQ ID NO: 55 - DNA, 1173 bp, Artificial
  cDNA encoding PPO3
atgaagctctcaacactaccagcagcactcctgtccttgattctcctggctcaccaggccgccgccgccccgctcggcaacgtcgacgctgaagcggccgcatcccagagaatcctcgagctgcaagctcagtaccagcgaaacgttctagacgccatcagcaaccgcacctcaggctgcacagcccaaaatatccagcgccgccaggaatggagcaccctctccctggctgaccgcgccgctttcatctcggcgatccactgcctcaacgcgctgccggcgcggacgccgcagtccaccgccccgggcgcccgggcgctctacgacgacttcatcgtcgcccacatcctacagactcccttcgtccacgcgtcgggcctcttcctgcccttccaccgccaccttctgcacctgttcgcaggcgcgctgcgcgaccgctgcgactacgccggcccgctgccctactgggactggacggcgagctacgccgacccgcgcgcggcggccgtcttcgacggcgggccgcactcgctcggcggcaacggcgccttcgtgcccggccgcaacggcaccgtcatctccgtccccggcggcgcgcacgtggtcatcccgcccgcgaccggcggcgggtgcgtcacgacggggccgttccgccagggccgcttcgaggtgcgcctcggccccgtcgcgttcgagccccgcggcccgcacggagggctgggctacaacccgcggtgcctccggcgggacctgagcccgcacttcagcctcggcacgcgccccggcgcggtcgtggccttgctggacggctgcgccggcgacctcggctgcctcgtgcgcgacatggacgcccccgggggcgtgcccgggggcgtgcacgcgtccgggcactggcaggtcgggccggacgcgctcgacgtcttcgccagcccgagcgaccccgtcttttggctgcaccacgcccaggtcgaccgcgtctggaccatctggcagggactggacctcgaggggaggacgtatcaggtctggggcacgagcacggcggcgaacgatccgccaagtgacgacgtaacgctcgagacggcaatggactttggcattcttggtgaggcaaagacggtcggtgaggttgcctcgacggtggacggagaatattgctacatgtatgagtga

SEQ ID NO: 56 - DNA, 1348 bp, Myceliophthora thermophila
atgaagctctcaacactaccagcagcactcctgtccttgattctcctggctcaccaggccgccgccgccccgctcggcaacgtcgacgctgaagcggccgcatcccagagaatcctcgagctgcaagctcagtaccagcgaaacgttctagacgccatcagcaaccgcacctcaggctgcacagcccaaaatatccagcgccgccaggaatggtacgttctccctttccctcattcttcccttcccttcgactccggacaaaacgccagagccccagtgtaagtacctaaacagatccttcaccgcgcaggagcaccctctccctggctgaccgcgccgctttcatctcggcgatccactgcctcaacgcgctgccggcgcggacgccgcagtccaccgccccgggcgcccgggcgctctacgacgacttcatcgtcgcccacatcctacagactcccttcgtccacgcgtcgggcctcttcctgcccttccaccgccaccttctgcacctgttcgcaggcgcgctgcgcgaccgctgcgactacgccggcccgctgccctactgggactggacggcgagctacgccgacccgcgcgcggcggccgtcttcgacggcgggccgcactcgctcggcggcaacggcgccttcgtgcccggccgcaacggcaccgtcatctccgtccccggcggcgcgcacgtggtcatcccgcccgcgaccggcggcgggtgcgtcacgacggggccgttccgccagggccgcttcgaggtgcgcctcggccccgtcgcgttcgagccccgcggcccgcacggagggctgggctacaacccgcggtgcctccggcgggacctgagcccgcacttcagcctcggcacgcgccccggcgcggtcgtggccttgctggacggctgcgccggcgacctcggctgcctcgtgcgcgacatggacgcccccgggggcgtgcccgggggcgtgcacgcgtccgggcactggcaggtcgggccggacgcgctcgacgtcttcgccagcccgagcgaccccgtcttttggctgcaccacgcccaggtcgaccgcgtctggaccatctggcagggactggacctcgaggggaggacgtatcaggtctggggcacgagcacggcggcgaacggtgagttcccttcccttccctttttttttttttttttttttcgttccctttgaacggatgatgctgacctgcttcagatccgccaagtgacgacgtaacgctcgagacggcaatggactttggcattcttggtgaggcaaagacggtcggtgaggttgcctcgacggtggacggagaatattgctacatgtatgagtga

SEQ ID NO: 57 - protein, 365 aa, Myceliophthora thermophila
TFNIPAFEQSAIDSGLALAGLNGIALLQSATRYGGSCNLGNVKIRREWRTLSKAQRKSYIKAVQCVQAKPSRLAEGIAAGSKTLFDDFVYVHMNATLFIHYTGNFLIWHRNFIHVYEKELNACGYKDALPYWEWGFDVEAPHLSPVFDGSDTSMSGDGEFVPGPPLELLVPGNAEPVVLAKGTGGGCVKTGPFANMTVRLGPITQPDPTADNPRCLKRDLNADAGKRFSSFRNTTNLIINSPTIEYFRTTMEGHPNYVPNSLGVHGGGHFMISGDPGADAFISPGDPAFYLHHAQIDRVYWIWQMLDFENRQGVFGTNTMLNYPPSENTTVEDTVDVGHLGGPIKIKNLMSTTGQNGSPLCYIYL

SEQ ID NO: 58 - protein, 382 aa, Myceliophthora thermophila
MKLSASLALAAAMGASATFNIPAFEQSAIDSGLALAGLNGIALLQSATRYGGSCNLGNVKIRREWRTLSKAQRKSYIKAVQCVQAKPSRLAEGIAAGSKTLFDDFVYVHMNATLFIHYTGNFLIWHRNFIHVYEKELNACGYKDALPYWEWGFDVEAPHLSPVFDGSDTSMSGDGEFVPGPPLELLVPGNAEPVVLAKGTGGGCVKTGPFANMTVRLGPITQPDPTADNPRCLKRDLNADAGKRFSSFRNTTNLIINSPTIEYFRTTMEGHPNYVPNSLGVHGGGHFMISGDPGADAFISPGDPAFYLHHAQIDRVYWIWQMLDFENRQGVFGTNTMLNYPPSENTTVEDTVDVGHLGGPIKIKNLMSTTGQNGSPLCYIYL

SEQ ID NO: 59 - DNA, 1149 bp, Artificial
  cDNA encoding PPO8
atgaagctctcggcgtctctggccttggctgcggccatgggggcctcggccaccttcaacattcccgcctttgagcagtccgctatcgactccggcctggctcttgctggtctgaacggcatcgccctgctgcagtcagccaccaggtacggcgggtcgtgcaatcttgggaacgtcaagatccgccgagaatggaggacgctgtccaaagcccagcggaagagttacatcaaggcggtgcagtgcgtccaagctaagcccagcagactggccgagggtattgccgctgggtccaagacgctcttcgatgactttgtttacgtacacatgaatgcgaccctcttcatccactacaccggcaacttcctcatctggcaccgcaacttcatccacgtgtacgagaaggagcttaacgcctgtggctacaaagatgccctcccctattgggaatggggcttcgatgtcgaggcgccacacctttcacctgtctttgacgggtctgatacatcgatgagtggcgatggcgagttcgtccctggccccccacttgagctcctggttcccggcaacgccgagcccgtggtactggcgaagggtactggtggcggctgtgtcaagacgggcccgtttgccaacatgacggtccggttgggcccaatcacgcagccggacccgaccgcggacaacccgcgctgcctgaagcgcgacctgaacgccgatgccggcaagcgcttctctagcttccgcaacacaacgaacctcattatcaactcgcccacgatcgagtatttccggaccaccatggagggccaccccaactatgtgcccaactcgctgggggtgcacggcggcggccacttcatgatcagcggcgacccgggcgcggatgccttcatctcgcccggcgaccccgcgttctaccttcatcatgcccaaatcgatcgcgtctactggatctggcagatgctggattttgagaaccgccagggcgtcttcggcaccaacactatgctcaactaccctcccagcgagaacaccaccgtcgaggacaccgtcgatgtgggccacctcggcggcccaatcaagatcaagaacctcatgagcactaccgggcaaaatggctctcctctttgctacatttatctttag

SEQ ID NO: 60 - DNA, 1345 bp, Myceliophthora thermophila
atgaagctctcggcgtctctggccttggctgcggccatgggggcctcggccaccttcaacattcccgcctttgagcagtccgctatcgactccggcctggctcttgctggtctgaacggcatcgccctgctgcagtcagccaccaggtacggcgggtcgtgcaatcttgggaacgtcaagatccgccgagaatggtatgtcgatcacccgtcgaccctgatctcctgcattccgcaacagctccgtgttgacccggggattaggaggacgctgtccaaagcccagcggaagagttacatcaaggcggtgcagtgcgtccaagctaagcccagcagactggccgagggtattgccgctgggtccaagacgctcttcgatgactttgtttacgtacacatgaatgcgaccctcttcatccactacaccgtgagcatccgccgatagttcgaggagtcctattgctaccgagacccccggcttacccttcctacagggcaacttcctcatctggcaccgcaacttcatccacgtgtacgagaaggagcttaacgcctgtggctacaaaggtattacgtgttcttgaaggtcgctatgtccgatgccacgtagctgatcaaggaatgcagatgccctcccctattgggaatggggcttcgatgtcgaggcgccacacctttcacctgtctttgacgggtctgatacatcgatgagtggcgatggcgagttcgtccctggccccccacttgagctcctggttcccggcaacgccgagcccgtggtactggcgaagggtactggtggcggctgtgtcaagacgggcccgtttgccaacatgacggtccggttgggcccaatcacgcagccggacccgaccgcggacaacccgcgctgcctgaagcgcgacctgaacgccgatgccggcaagcgcttctctagcttccgcaacacaacgaacctcattatcaactcgcccacgatcgagtatttccggaccaccatggagggccaccccaactatgtgcccaactcgctgggggtgcacggcggcggccacttcatgatcagcggcgacccgggcgcggatgccttcatctcgcccggcgaccccgcgttctaccttcatcatgcccaaatcgatcgcgtctactggatctggcagatgctggattttgagaaccgccagggcgtcttcggcaccaacactatgctcaactaccctcccagcgagaacaccaccgtcgaggacaccgtcgatgtgggccacctcggcggcccaatcaagatcaagaacctcatgagcactaccgggcaaaatggctctcctctttgctacatttatctttag

SEQ ID NO: 61 - protein, 373 aa, Myceliophthora thermophila
SVPAAGNLDIVKNLAHRMGEEDMDAFPVLSWDEAVAIMTGNDEGGGSSSSSSSGSGRGPDNNRVSVNNYSNKTKPAYPPAYVPHASSCGKPKVRVEWKKLKESEKFAYLGAVKCLMDTKPVGVWSKATSIWDEMAWAHNEAKWNIHETDNFLPWHRYYIYMLETLLSKHCSYRGPIPWWRETDDTGNLAGANLFSPNYFGSLPPRTEDGKTTCITDGWFANTTVLLGPGAPTCLARGEDKRIAAEVTTAALDLCQGDRDTKYERHRRCVEATIHSSTHRAVGGAMESISASPSDPVFYLHHGFVDWQWARWQNVESSRKTTISGCSEPSPVPGKCVELTLDTVLKGYGLIPDMKVRDVLDIENGILCYTYDEF

SEQ ID NO: 62 - protein, 392 aa, Myceliophthora thermophila
MRLTRYAPVLSAAVLAAAASVPAAGNLDIVKNLAHRMGEEDMDAFPVLSWDEAVAIMTGNDEGGGSSSSSSSGSGRGPDNNRVSVNNYSNKTKPAYPPAYVPHASSCGKPKVRVEWKKLKESEKFAYLGAVKCLMDTKPVGVWSKATSIWDEMAWAHNEAKWNIHETDNFLPWHRYYIYMLETLLSKHCSYRGPIPWWRETDDTGNLAGANLFSPNYFGSLPPRTEDGKTTCITDGWFANTTVLLGPGAPTCLARGEDKRIAAEVTTAALDLCQGDRDTKYERHRRCVEATIHSSTHRAVGGAMESISASPSDPVFYLHHGFVDWQWARWQNVESSRKTTISGCSEPSPVPGKCVELTLDTVLKGYGLIPDMKVRDVLDIENGILCYTYDEF

SEQ ID NO: 63 - DNA, 1179 bp, Artificial
  cDNA encoding PPO5
atgaggctcaccaggtatgccccggtgctgtcggcagccgtgttggcagcagcggcctcagtcccggcagccgggaatctcgacattgtcaagaatctggctcatcgcatgggtgaagaggacatggacgcgttcccagtgttatcgtgggacgaggctgtggccatcatgacggggaatgacgagggcggcggcagcagcagcagcagcagcagcggtagcggtcgcggtcccgacaacaacagggtctcggtcaacaactacagcaacaagaccaagcccgcctacccccccgcctacgtcccccacgcgtcctcctgcgggaagcccaaggtccgggtcgagtggaagaagctgaaagagtcggaaaagtttgcgtacctgggggcggtcaagtgcctgatggacacgaagccggtcggcgtctggtccaaggcgacgagcatctgggacgagatggcatgggcgcacaacgaggccaaatggaacatccacgagaccgacaacttcctgccctggcaccgctactacatctacatgctcgagacgctcctcagcaagcactgctcctaccgggggcccatcccctggtggcgcgagaccgacgacaccggcaacttggccggggccaatctgttttcgcccaactacttcggctcgctacccccccggacggaagacggtaagaccacatgcattaccgacggctggtttgccaacaccacggtccttctcgggcccggcgctccgacttgcctcgcccggggcgaggataagagaatcgccgccgaggtgacgacggccgccctggacctgtgccaaggcgacagagacaccaagtacgagaggcaccgtcgctgcgttgaggccaccatccattcctccacgcaccgggccgtcggcggcgcaatggaaagcatcagcgcctccccgagcgaccccgtcttctacctccaccacggtttcgtcgactggcagtgggcgcgctggcagaacgtcgaatccagccgcaagaccacaatttccggctgctccgagccttccccggtccctggcaaatgtgtggagctcaccctagacacggtactgaagggatatggcttgatacccgacatgaaggtccgcgacgttctcgacatcgaaaacggaatcctgtgctacacctatgacgagttttga

SEQ ID NO: 64 - DNA, 1434 bp, Myceliophthora thermophila
atgaggctcaccaggtatgccccggtgctgtcggcagccgtgttggcagcagcggcctcagtcccggcagccgggaatctcgacattgtcaagaatctggctcatcgcatgggtgaagaggacatggacgcgttcccagtgttatcgtgggacgaggctgtggccatcatgacggggaatgacgagggcggcggcagcagcagcagcagcagcagcggtagcggtcgcggtcccgacaacaacagggtctcggtcaacaactacagcaacaagaccaagcccgcctacccccccgcctacgtcccccacgcgtcctcctgcgggaagcccaaggtccgggtcgagtggaagaagctgaaagagtcggaaaagtttgcgtacctgggggcggtcaagtgcctgatggacacgaagccggtcggcgtctggtccaaggcgacgagcatctgggacgagatggcatgggcgcacaacgaggccaaatggaacatccacgagaccgacaacttcctgccctggcaccgctactacatctacatgctcgagacgctcctcagcaagcactgctcctaccgggggcccatcccctggtggcgcgagaccgacgacaccggcaacttggccggggccaatctgttttcgcccaactacttcggctcgctacccccccggacggaagacggtaagaccacatgcattaccgacggcgtaagtataagatatcctctttgattatacttgtttctttctcttttcttccctctcttcttctctggtactttcaagcataaccactcggcatccgctaattaattaatcgatccccggtaacaaccttgaaagtggtttgccaacaccacggtccttctcgggcccggcgctccgacttgcctcgcccggggcgaggataagagaatcgccgccgaggtgacgacggccgccctggacctgtgccaaggcgacagagacaccaagtacgagaggcaccgtcgctgcgttgaggccaccatccattcctccacgcaccgggccgtcggcggcgcaatggaaagcatcagcgcctccccgagcgaccccgtcttctacctccaccacggtttcgtcgactggcagtgggcgcgctggcagaacgtcgaatccagccgcaagaccacaagtaagtctgatttcctgtttccatgcgggaaatctttaaaatttagtgaaattaagggggggaaaaaggaagacctatgtgtgagagaacatcctgactaatacctttcgtgtcacacagtttccggctgctccgagccttccccggtccctggcaaatgtgtggagctcaccctagacacggtactgaagggatatggcttgatacccgacatgaaggtccgcgacgttctcgacatcgaaaacggaatcctgtgctacacctatgacgagttttga

SEQ ID NO: 65 - protein, 271 aa, Myceliophthora thermophila
TNPAKRRAWHTLSNDEKQVYIQAELCLMELPAKLSFPAARTRFDELQVIHQLQAYATHFVVSEDEAISGTTKLSCPLTCFLCVRGRPRRRGWAGESKFMDLLAFFVPFTFTFSSGFLHLFGNPLLAIRSPRAMVVLASRSFGSLEGDRSKGYLLTLSMLNGVSSPGDPLFYLHNTWLDKVFWDWQARDRSTRTTTIGGTNIAPDAPPGFPTRPSNIPLPAGADGNPGKTTTLSHVLNMYGNMPNATVGDVLDIQGDFVCYEYVEPEEEVTV

SEQ ID NO: 66 - protein, 292 aa, Myceliophthora thermophila
MARFSKLAVGLLMVSLASVACTNPAKRRAWHTLSNDEKQVYIQAELCLMELPAKLSFPAARTRFDELQVIHQLQAYATHFVVSEDEAISGTTKLSCPLTCFLCVRGRPRRRGWAGESKFMDLLAFFVPFTFTFSSGFLHLFGNPLLAIRSPRAMVVLASRSFGSLEGDRSKGYLLTLSMLNGVSSPGDPLFYLHNTWLDKVFWDWQARDRSTRTTTIGGTNIAPDAPPGFPTRPSNIPLPAGADGNPGKTTTLSHVLNMYGNMPNATVGDVLDIQGDFVCYEYVEPEEEVTV

SEQ ID NO: 67 - DNA, 879 bp, Artificial
  cDNA encoding PPO10
atggctcgattctctaagcttgccgtcggcctcctcatggtgtctttggcttctgtggcctgcaccaacccggccaaacgcagggcatggcataccctgagcaacgatgagaagcaggtgtacatccaggccgagctttgtctcatggagctgcccgccaagctgagcttcccggcggcccggacgaggttcgacgagctccaagtcatccaccagctccaggcatatgccacccatttcgtggtgagtgaagatgaggctatttcagggacgacaaagttatcttgcccgctgacgtgctttctctgcgtcagggggcggccacggcggcgtgggtgggcaggtgagtctaagtttatggatctgcttgcgttctttgttccttttacctttactttttcgtctggtttcctacatctgttcggaaatccactacttgctatcagatcccctcgtgctatggtggtgttggcaagtcgcagtttcggtagtctcgagggcgatcgatcgaaaggatatttgctgaccttatccatgctcaacggtgtctcgagccccggcgaccctctattctacctacacaacacatggctggacaaggtgttctgggactggcaggccagggacaggtcgacgcgcacgacgaccatcggcgggaccaacatcgccccggacgcgccgccgggcttccccacgcggccctcgaatatcccgttacctgctggtgccgacggcaaccctggcaaaaccaccacgctaagccatgtgctcaacatgtacggcaatatgcccaacgcgacggtgggtgacgtcttggacattcagggcgactttgtctgctacgagtacgtcgagccagaggaggaggtgacggtatga

SEQ ID NO: 68 - DNA, 1072 bp, Myceliophthora thermophila
atggctcgattctctaagcttgccgtcggcctcctcatggtgtctttggcttctgtggcctgcaccaacccggccaaacgcagggcatggtaagcaggatatctgcccggatccatcgttgctcgacacgactaactgactgactgactgattcgtctttgggttgggagggcaggcataccctgagcaacgatgagaagcaggtgtacatccaggccgagctttgtctcatggagctgcccgccaagctgagcttcccggcggcccggacgaggttcgacgagctccaagtcatccaccagctccaggcatatgccacccatttcgtggtgagtgaagatgaggctatttcagggacgacaaagttatcttgcccgctgacgtgctttctctgcgtcagggggcggccacggcggcgtgggtgggcaggtgagtctaagtttatggatctgcttgcgttctttgttccttttacctttactttttcgtctggtttcctacatctgttcggaaatccactacttgctatcagatcccctcgtgctatggtggtgttggcaagtcgcagtttcggtagtctcgagggcgatcgatcgaaaggatatttgctgaccttatccgtcggggcgaaatccgactcatagatgctcaacggtgtctcgagccccggcgaccctctattctacctacacaacacatggctggacaaggtgttctgggactggcaggccagggacaggtcgacgcgcacgacgaccatcggcgggaccaacatcgccccggacgcgccgccgggcttccccacgcggccctcgaatatcccgttagtctgcccctgactgatatcctcctcatgcttagggtcgacaattcaatggcgttcggcgacactgattgtatatctagcaagcctgctggtgccgacggcaaccctggcaaaaccaccacgctaagccatgtgctcaacatgtacggcaatatgcccaacgcgacggtgggtgacgtcttggacattcagggcgactttgtctgctacgagtacgtcgagccagaggaggaggtgacggtatga

SEQ ID NO: 69 - protein, 336 aa, Myceliophthora thermophila
QTYPPDVVDQLAKDSLPKLKEWLERNPQEGCTYETAVKRREWRDLTVEERKAYTDAVLCLQSKPALTSDQAPGAKSRFDDYVVIHIQQTPRNHGSYWNWDRYAKDPANSPLFDGSEGSMGSNGAKEEHQGIPIPGAPPPYNMIPPGDGGGCVTSGPFKNMTVNVGPIAPTLNVQRNPRADGLGYNPRCLRRDINKHSAAVTTANYTYDLITNNHEIYWFQTVMEGQFPQGKWGVHAGGHYTVSGDPAGDFYVSPGDPVFWLHHAMIDRVWWIWQLQDLDSRLTQVSMTKTMNNFPPSENGTLDDLSGLGVLAPDVTVRELMNTMGGIGGKFCYIYE

SEQ ID NO: 70 - protein, 354 aa, Myceliophthora thermophila
MVLSKVWGATLLAGFAVAQTYPPDVVDQLAKDSLPKLKEWLERNPQEGCTYETAVKRREWRDLTVEERKAYTDAVLCLQSKPALTSDQAPGAKSRFDDYVVIHIQQTPRNHGSYWNWDRYAKDPANSPLFDGSEGSMGSNGAKEEHQGIPIPGAPPPYNMIPPGDGGGCVTSGPFKNMTVNVGPIAPTLNVQRNPRADGLGYNPRCLRRDINKHSAAVTTANYTYDLITNNHEIYWFQTVMEGQFPQGKWGVHAGGHYTVSGDPAGDFYVSPGDPVFWLHHAMIDRVWWIWQLQDLDSRLTQVSMTKTMNNFPPSENGTLDDLSGLGVLAPDVTVRELMNTMGGIGGKFCYIYE

SEQ ID NO: 71 - DNA, 1065 bp, Artificial
  cDNA encoding PPO9
atggttctgtcaaaagtctggggagcgaccctcctcgctggttttgcggtcgcccaaacgtaccctccagatgtggttgatcagctggcgaaggatagtttgcccaagctcaaagaatggctcgagaggaacccacaggagggatgtacctacgagacggcagtcaagcggcgggaatggagggacttgaccgtcgaggaacgcaaggcatacacggatgccgtcctctgcctccagtcgaagcctgccttgacaagtgaccaagcgccgggagccaagagcaggtttgacgattatgtcgtcatccatatccaacaaacaccgaggaaccatggctcgtactggaactgggatcgatatgctaaagaccccgccaactcgccgctcttcgacggcagcgagggcagcatgggtagcaacggtgccaaggaagaacaccagggcattcctatccccggtgcgccgcctccgtacaacatgatcccgcccggggacggcggtggctgcgtgacctcgggcccctttaagaacatgaccgtcaacgtcggcccaatcgcgccgacgctgaacgtgcagcgcaacccgcgggcggacgggctggggtacaacccacggtgcctgcggcgcgacatcaacaagcactccgcggcggtgacgacggccaactacacgtacgacctcatcaccaacaaccacgagatctactggttccagacggtcatggagggccagttcccgcaaggcaagtggggagtgcacgcgggagggcactacaccgtctcgggcgaccccgcaggcgacttctacgtcagccccggcgaccccgtcttctggctgcaccacgccatgatcgaccgcgtctggtggatctggcagctccaggacctcgacagccgcctgacccaggtgagcatgaccaagaccatgaacaacttcccccccagcgagaacggcaccctcgacgacctcagcggcctcggcgtgctcgccccggacgtcaccgtccgggagctcatgaacaccatgggagggatcggcggtaagttttgctacatctacgagtaa

SEQ ID NO: 72 - DNA, 1517 bp, Myceliophthora thermophila
atggttctgtcaaaagtctggggagcgaccctcctcgctggttttgcggtcgcccaaacgtaccctccagatgtggttgatcagctggcgaaggatagtttgcccaagctcaaagaatggctcgagaggaacccacaggagggatgtacctacgagacggcagtcaagcggcgggaatggtttgctgcaccctcccgggttcctcacgagctgaactctgacatagcccaggagggacttgaccgtcgaggaacgcaaggcatacacggatgccgtcctctgcctccagtcgaagcctgccttgacaagtgaccaagcgccgggagccaagagcaggtttgacgattatggtacacctcccgatctcagcccgacagataatagaatacaccctactaacggcgttgcatcaaagtcgtcatccatatccaacaaacaccgaggaaccatggctcggtaagccggcaacttcgctgctccctcagcgccccacgacgccaccatctaacatccagcagaccttcttcctcccgtggcatcggtactacgtctggcactacgagaacgcgctgagaaccgagtgcggctacaaaggatatcagccggtaagaccacatcgccttcgcatatacgacaggacagagccgccgctgacaagggtggggtttgaagtactggaactgggatcgatatgctaaagaccccgccaactcgccgctcttcgacggcagcgagggcagcatgggtagcaacggtgccaaggaagaacaccagggcattcctatccccggtgcgccgcctccgtacaacatgatcccgcccggggacggcggtggctgcgtgacctcgggcccctttaagaagtgagagcctttgactattaagtcccaaatccctgtcaaaaacccccatttcttgcagcacctatgaagacggaagcgtattatttactgacccccccttttcccttcctccaacccagcatgaccgtcaacgtcggcccaatcgcgccgacgctgaacgtgcagcgcaacccgcgggcggacgggctggggtacaacccacggtgcctgcggcgcgacatcaacaagcactccgcggcggtgacgacggccaactacacgtacgacctcatcaccaacaaccacgagatctactggttccagacggtcatggagggccagttcccgcaaggcaagtggggagtgcacgcgggagggcactacaccgtctcgggcgaccccgcaggcgacttctacgtcagccccggcgaccccgtcttctggctgcaccacgccatgatcgaccgcgtctggtggatctggcagctccaggacctcgacagccgcctgacccaggtgagcatgaccaagaccatgaacaacttcccccccagcgagaacggcaccctcgacgacctcagcggcctcggcgtgctcgccccggacgtcaccgtccgggagctcatgaacaccatgggagggatcggcggtaagttttgctacatctacgagtaa

SEQ ID NO: 73 - protein, 837 aa, Clostridium thermocellum
MSRKLFSVLLVGLMLMTSLLVTISSTSAASLPTMPPSGYDQVRNGVPRGQVVNISYFSTATNSTRPARVYLPPGYSKDKKYSVLYLLHGIGGSENDWFEGGGRANVIADNLIAEGKIKPLIIVTPNTNAAGPGIADGYENFTKDLLNSLIPYIESNYSVYTDREHRAIAGLSMGGGQSFNIGLTNLDKFAYIGPISAAPNTYPNERLFPDGGKAAREKLKLLFIACGTNDSLIGFGQRVHEYCVANNINHVYWLIQGGGHDFNVWKPGLWNFLQMADEAGLTRDGNTPVPTPSPKPANTRIEAEDYDGINSSSIEIIGVPPEGGRGIGYITSGDYLVYKSIDFGNGATSFKAKVANANTSNIELRLNGPNGTLIGTLSVKSTGDWNTYEEQTCSISKVTGINDLYLVFKGPVNIDWFTFGVESSSTGLGDLNGDGNINSSDLQALKRHLLGISPLTGEALLRADVNRSGKVDSTDYSVLKRYILRIITEFPGQGDVQTPNPSVTPTQTPIPTISGNALRDYAEARGIKIGTCVNYPFYNNSDPTYNSILQREFSMVVCENEMKFDALQPRQNVFDFSKGDQLLAFAERNGMQMRGHTLIWHNQNPSWLTNGNWNRDSLLAVMKNHITTVMTHYKGKIVEWDVANECMDDSGNGLRSSIWRNVIGQDYLDYAFRYAREADPDALLFYNDYNIEDLGPKSNAVFNMIKSMKERGVPIDGVGFQCHFINGMSPEYLASIDQNIKRYAEIGVIVSFTEIDIRIPQSENPATAFQVQANNYKELMKICLANPNCNTFVMWGFTDKYTWIPGTFPGYGNPLIYDSNYNPKPAYNAIKEALMGY

SEQ ID NO: 74 - protein, 290 aa, Neurospora crassa
MAGLHSRLTTFLLLLLSALPAIAAAAPSSGCGKGPTLRNGQTVTTNINGKSRRYTVRLPDNYNQNNPYRLIFLWHPLGSSMQKIIQGEDPNRGGVLPYYGLPPLDTSKSAIYVVPDGLNAGWANQNGEDVSFFDNILQTVSDGLCIDTNLVFSTGFSYGGGMSFSLACSRANKVRAVAVISGAQLSGCAGGNDPVAYYAQHGTSDGVLNVAMGRQLRDRFVRNNGCQPANGEVQPGSGGRSTRVEYQGCQQGKDVVWVVHGGDHNPSQRDPGQNDPFAPRNTWEFFSRFN

SEQ ID NO: 75 - protein, 530 aa, Talaromyces stipitatus
MMLTSAILLLTLGVQLSHADDSSRENFSNRCDQLAKEIHIPNVTVNFVEYVANGTNVTLADNPPSCGQSNQVVLADLCRVAMEVTTSNQSQITLEAWFPENYTGRFLSTGNGGLAGCIQYVDMAYASSMGFATVGANGGHNGTSGESFYHNPDIVEDLSWRSVHTGVVVGKELTKKFYHEGFHKSYYLGCSTGGRQGFKAVQEFVHDFDGVVAGCPAFNFVNLNSWSGHFYPITGNSSADTFLTTAQWTLVQQSVMEQCDSLDGAVDGVIEAIDQCHPVFEQLICRPGQNASECLTGKQVNTAQLVLSPIYGTKGEFLYPRMQPGVENVDMYITYNGDPFAYSTDWYKYVVFSDPNWDPATLNAQDYEIALAQNPSNIQTFEGDLSAFRDAGAKVLTYHGTADPIITGETSKVYYRHVAETMNAAPEELDEFYRYFRIGGMSHCGGGTGATAIGNVLSAQWSNDPDANVLMAMVRWVEEGVAPEYIRGASLGSGPGAKVEYTRRHCKYPTRNVYVGPGNWTDENAWKCIL

SEQ ID NO: 76 - protein, 585 aa, Thielavia terrestris
MAAFAKLTTRALSLSDICTVSNVQAALPSNGTLLGIDLIPSSVTASVTNGTSGGSMGMGGGSSPGSSITYCSVTVTYTHTGKGDTVNLKYAFPSPSDFQNRFYVAGGGGFSLSSDATGGLSYGAAGGATDAGYDAFSNSYDKVVLYGNGSINWDATYMFGYQALGEMTKVGKVLTKGFYGLSDDTKIYTYFEGCSDGGREGMSQVQRWGDEYGGVIAGAPAFRFAQQQVLHVLSSTVEKMLDYLPPPCELAKIVNATIAACDGLDGRSDGVVSRTDLCKLKFNLKSVIGESYYCAAKNSTSLGSGFNKAKRQAPGSQTSYQLEQSGTVSAKGVAVAQAIYDGLHNSAGERAYLSWQIASELADASTTYNSAIGAWELNIPSIGGEYVTKFVQLLDLDNLPSLDNVTYDTLVDWMTTGMYRYMDSLQTTLPDLTTFQSSGGKLLHYHGESDSSIPAASSVHYWQSVRSVMYPSLSDEQSLAALADWYQLYLVPGAGHCGANPLQPGPFPRNNMNVMIDWVENGVRPSRLNATVSSGKYAGETQMLCQWPTRPLWRSNDSSTFDCVTDAASYESWTYTFPAFKVPVY

SEQ ID NO: 77 - protein, 287 aa, Humicola insolens
MRFSTILSVAATASTALGASLQQVWNWGANPTNIQFHIYVPDRRAANPAIIVALHPCGGNAQQWFGGTRLPQYADQHGFILIYLSTPNQSNCWDVHNTASLTHNGGGDALGIVSMVNYTIDRYNADRSRVYVMGFSSGGMMTNVLAGSYPEVFEAGAAYSGTAHACFFGEPFSPNQTCAQGLSHTPEQWGNFVRNSYPGYNGRRPRMQIVHGLQDFLVRPQCGYEALKQWSNVLGIPFTRQVQNSPSWGWTTELYGDGTQLQGLFSQNLGHAPAVDEQQLLRFFRLI

SEQ ID NO: 78 - protein, 789 aa, Ruminococcus sp.
VKKTVKQFISSAVTALMVAASLPAVPSVNAADAQQRGNIGGFDYEMWNQNGQGQVSMTPKAGSFTCSWSNIENFLARMGKNYDSQKKNYKAFGDITLSYDVEYTPKGNSYMCVYGWTRNPLMEYYIVEGWGDWRPPGNDGENKGTVTLNGNTYDIRKTMRYNQPSLDGTATFPQYWSVRQKSGSQNNTTNYMKGTISVSKHFDAWSKAGLDMSGTLYEVSLNIEGYRSSGNANVKAISFDGSIPEPTSEPVTQPVVKAEPDANGYYFKEKFESGAGDWSARGTGAKVTSSDGFNGSKGILVSGRGDNWHGAQLTLDSSAFTAGETYSFGALVKQDGESSTAMKLTLQYNDASGTANYDKVAEFTAPKGEWVDLSNTSFTIPSGASDLILYVEAPDSLTDFYIDNAFGGIKNTSPLEDVGSHTISTPGSETTTVTTASNKGIRGDINGDGVINSFDLAPLRRGILKMMSGSGSTPENADVNGDGTVNVADLLLLQKFILGMEKSFPDPVTTTTTKPITTTTEKIVTTTTSSSSSSSGKNLNADIRKDMPTSVPGGNEKSGGCKVEKKTYNCKFTGGQKSCNVILPPNYSASKQYPVMYVLHGIGGNEGSMVSGMGVQELLAGLTANGKAEEMIIVLPSQYTSKNGNQGGGFGINQEVCAAYDNFLYDISDSLIPFIEANYPVKTGRENRAITGFSMGGREAIYIGLMRPDLFAYVGGACPAPGITPGKDMFMEHPGCMQESEMKFRDVGPEPNVFMITGGTNDGVVGTFPKQYSDILTRNGVDQRLPVYP

SEQ ID NO: 79 - protein, 530 aa, Orpinomyces sp.
VVSCETTYGITLRDTKEKFTVFKDGSAATDIVESEDGSVSWIATAAGGAGGGVAFYVKANKEEINIANYESIDIEMEYTPVENKWNDAAKNPSFCMRILPWDSTGMFGGYEDLEYFDTPAKSGNFKYTIKIPSFFADKILSSSDLDSILSFAIKFNDYERGNTDGDQIKIQLKNVKFNPKENAPEDKAFDDGLRDSQRGTVVEMKYSSRDYTVKESEADKYEKHAWVYLPAGYEADNKDKKYPLVVLLHGYGQNENTWGLSNKGRGGKIKGYMDRGMASGNVEKFVLVAATGVASKNWGPNGSGVDLDGFNAFGGELRNDLLPYIRAHFNVKVDRDHTALAGLSMGGGQTISIGIGETLDEISNYGSFSPALFQTAEEFFGKVKGNFKEELRIHNLYMTCGDADTLVYDTYPSYVEALKNWDAVEFMKEYTYPGGTHDFPVWYRGFNEFIQIVFKNQKVKEEPIHADPVEDPSDEPVSVDPSVSVEEPNDSESSSEDEPVVKKTIKHTIAKKKPSKTRTVTKKVIKKKNN

SEQ ID NO: 80 - protein, 499 aa, Schizophyllum commune
QGDFSANCSSFVDAITLENVTVQSTEFVAAGTNVSVYVPESCMSASYQVVSADLCRATMNVSTSDRSGIRLEAWFPQNYTGRFLSTGNGGIGGCIQYSDLDYAASLGFAAVGANNGHDGMTGEPFLNNPDVITDFAWRSLHTGVVVGKQLVETFYGAPHSKSYYLGCSTGGRQGWKMVQDFPEDFDGVVAGAPAMAFNALLYWSGSFYTVTGTSDDPTFVPVDFWANNIHQAVLDQCDALDGATDGVLENPDLCDFDVTPLNCADGNTTDCLTETQAETVRTVLSAIYDANGTLVYPRLQPGAEVLAGQQQFNGQPFDITQDWYRYVIYNDSTWDPATLNLGDYAVALAQNPANIETFNGDISAFEAAGGKVLHYHGLMDGLISSDNSKRYYKLVQETMGKDSSELDDFYRFFPISGMSHCSGGDGAYRIGNVDGGAGTSADDNVLMSMVRWVEEGVAPEVVRGANANVTYWRSHCKWPLTNKYVGPGSYEDESAWQCS

SEQ ID NO: 81 - protein, 499 aa, Schizophyllum commune
QDDFASDCSAFIDKIILDNVTVTSTEFVAAGTNLTVYVPNSCGSPSYQVVSTDLCRATMNVTTSERSGIRLEAWFPQNYTGRFLSTGNGGIGGCIQYSDIDYAASLGFAAVGANNGHDGMTGEPFLNNPDVVTDFAWRSLHTGVVVGKQLTETFYGAPHNKSYYLGCSTGGRQGFKAVQDFPDDFDGVVAGAPAFAFNGLLYWSGSFLPVTGTSEDPTFVPTDFWAQNIHAEVLQQCDALDGATDGVLENPDLCDFDVTPLICTDGNTTGCLTETQAETVRTVLSPIYDKNGTLVYPRLQPGAEVLAGFAQFGGQPFLYSTDWYRYVVYNDSNWDPMTLSIDDYAAARAQNPANVETFEGDISAFASSGGKVLHYHGLMDGLISSDNSKRYYALVQQTLNQEPAELDEFYRFFPISGMSHCSGGDGAYRIGNVEGGAGTSADDNVLMSMVRWVEEGVAPEVVRGADANATYWRAHCKWPKTNKYVGPGDYEDESAWECS

SEQ ID NO: 82 - protein, 526 aa, Aspergillus oryzae
MPSLRRLLPFLAAGSAALASQDTFQGKCTGFADKINLPNVRVNFVNYVPGGTNLSLPDNPTSCGTTSQVVSEDVCRIAMAVATSNSSEITLEAWLPQNYTGRFLSTGNGGLSGCIQYYDLAYTSGLGFATVGANSGHNGTSGEPFYHHPEVLEDFVHRSVHTGVVVGKQLTKLFYEEGFKKSYYLGCSTGGRQGFKSVQKYPNDFDGVVAGAPAFNMINLMSWSAHFYSITGPVGSDTYLSPDLWNITHKEILRQCDGIDGAEDGIIEDPSLCSPVLEAIICKPGQNTTECLTGKQAHTVREIFSPLYGVNGTLLYPRMQPGSEVMASSIMYNGQPFQYSADWYRYVVYENPNWDATKFSVRDAAVALKQNPFNLQTWDADISSFRKAGGKVLTYHGLMDQLISSENSKLYYARVAETMNVPPEELDEFYRFFQISGMAHCSGGDGAYGIGNQLVTYNDANPENNVLMAMVQWVEKGIAPETIRGAKFTNGTGSAVEYTRKHCRYPRRNVYKGPGNYTDENAWQCV

SEQ ID NO: 83 - protein, 281 aa, Aspergillus niger
MKQFSAKYALILLATAGQALAASTQGISEDLYNRLVEMATISQAAYADLCNIPSTIIKGEKIYNAQTDINGWILRDDTSKEIITVFRGTGSDTNLQLDTNYTLTPFDTLPQCNDCEVHGGYYIGWISVQDQVESLVKQQASQYPDYALTVTGHSLGASMAALTAAQLSATYDNVRLYTFGEPRSGNQAFASYMNDAFQVSSPETTQYFRVTHSNDGIPNLPPADEGYAHGGVEYWSVDPYSAQNTFVCTGDEVQCCEAQGGQGVNDAHTTYFGMTSGACTW

SEQ ID NO: 84 - protein, 521 aa, Aspergillus niger
MKVASLLSLALPGAALAATDPFQSRCNEFQNKIDIANVTVRSVAYVAAGQNISQAEVASVCKASVQASVDLCRVTMNISTSDRSHLWAEAWLPRNYTGRFVSTGNGGLAGCVQETDLNFAANFGFATVGTNGGHDGDTAKYFLNNSEVLADFAYRSVHEGTVVGKQLTQLFYDEGYNYSYYLGCSTGGRQGYQQVQRFPDDYDGVIAGSAAMNFINLISWGAFLWKATGLADDPDFISANLWSVIHQEIVRQCDLVDGALDGIIEDPDFCAPVIERLICDGTTNGTSCITGAQAAKVNRALSDFYGPDGTVYYPRLNYGGEADSASLYFTGSMYSRTEEWYKYVVYNDTNWNSSQWTLESAKLALEQNPFNIQAFDPNITAFRDRGGKLLSYHGTQDPIISSTDSKLYYRRVANALNAAPSELDEFYRFFQISGMGHCGDGTGASYIGQGYGTYTSKAPQVNLLRTMVDWVENGKAPEYMPGNKLNANGSIEYMRKHCRYPKHNIHTGPGNYTDPNSWTCV

SEQ ID NO: 85 - protein, 270 aa, Emericella nidulans
MLRAVLLPTLLAFGAFTPVHGANSPGCGKQPTLTNGVNQINGREYVLKIPDGYDPSKPHHLIFGLHWRGGNMYNVVNGDSIQPWYGLEARAQGSAIFVAPNGLNAGWANTNGEDVAFIDAIMEQVEDDLCVDQASRFATGFSWGGGMSYALACARAAEFRAVSVLSGGLISGCDGGNDPIAYLGIHGINDPVLPLDGGVTLANTFVSNNGCQPTDIGQPASGSGGSVRTDFSGCSHPVSFIAYDGGHDGAPLGVGSSLAPDATWEFFMAA

SEQ ID NO: 86 - protein, 558 aa, Fusarium oxysporum
MLFASLVLVLGFIPQVLSDTSTDICLPQDNMRPTFLLFSGLGACAAAGKGDDFAAKCAGFKTSLKLPNTKVWFTEHVPAGKNITFPDNHPTCTPKSTITDVEICRVAMFVTTGPKSNLTLEAWLPSNWTGRFLSTGNGGMAGCIQYDDVAYGAGFGFATVGANNGHNGTSAVSMYKNSGVVEDYVYRSVHTGTVLGKELTKKFYGKKHTKSYYLGCSTGGRQGWKEAQSFPDDFDGIVAGAPAMRFNGLQSRSGSFWGITGPPGAPTHLSPEEWAMVQKNVLVQCDEPLDGVADGILEDPNLCQYRPEALVCSKGQTKNCLTGPQIETVRKVFGPLYGNNGTYIYPRIPPGADQGFGFAIGEQPFPYSTEWFQYVIWNDTKWDPNTIGPNDYQKASEVNPFNVETWEGDLSKFRKRGSKIIHWHGLEDGLISSDNSMEYYNHVSATMGLSNTELDEFYRYFRVSGCGHCSGGIGANRIGNNRANLGGKEAKNNVLLALVKWVEEDQAPETITGVRYVNGATTGKVEVERRHCRYPYRNVWDRKGNYKNPDSWKCELPK

SEQ ID NO: 87 - protein, 279 aa, Myceliophthora thermophila
MISVPALALALLAAVQVVESASAGCGKAPPSSGTKSMTVNGKQRQYILQLPNNYDANKAHRVVIGYHWRDGSMNDVANGGFYDLRSRAGDSTIFVAPNGLNAGWANVGGEDITFTDQIVDMLKNDLCVDETQFFATGWSYGGAMSHSVACSRPDVFKAVAVIAGAQLSGCAGGTTPVAYLGIHGAADNVLPIDLGRQLRDKWLQTNGCNYQGAQDPAPGQQAHIKTTYSCSRAPVTWIGHGGGHVPDPTGNNGVKFAPQETWDFFDAAVGAAGAQSPMT

SEQ ID NO: 88 - protein, 302 aa, Myceliophthora thermophila
MSRYLLGRLIVATVAITVASLRGVTAATPSPGCGKTPTLITDGSATTPLTLTSNGKTRRFYVKLPDDYDNSHPYRLIFALHALGGTAQQVTTGTGGYLPWYGIPDLAANDTVGAVYVAPDGLNNGWANQGGEDVAFLEAVMETVEQDVCVDRDLRFSTGFSYGAAMSYTLACALGRRIRAVAVLSGSPVISGGCAGAGSGASEPVAYYGQHGMSDPVLPVAGGREMRDHFVRTNGCDAGRGPPREPARGSGTHVKTVYDGCDPDYPVVWNAFDGDHTPQPVDRGATTTFSAVETWEFFSQFK

SEQ ID NO: 89 - protein, 319 aa, Myceliophthora thermophila
MPCKSSLLLLLLSSFFSSEANTAHRPPCVSAIAHQDGRHGRPARGASLQLVTNFGDNPTGLQKYVYVPDKVAVSPAIIVALHPCGGSAQGWYSQTRLPSYADQLGFILIYAGTTKMSNCWDVQNPASLTHNGGGDAGGIVSMVKYALKQYNGDASRVYVMGGSSGAMMTNVLAGSYPDVFEAGAAFSGVAHACFLGADSATPFSPQTCAQGRIQRSAREWGDLVRNSFPAYDGRRPRMQIFHGNADFLVHPECAHQALAQWADVLGLQLTQTNKGVPSAEYTQEVYGDGTQLQGFFGDGVGHIAPVNEPVMLRFFGLMN

SEQ ID NO: 90 - protein, 291 aa, Myceliophthora thermophila
MLVRSFLGFAVLAATCLAASLQEVTEFGDNPTNIQMYIYVPDQLDTNPPVIVALHPCGGSAQQWFSGTQLPSYADDNGFILIYPSTPHMSNCWDIQNPDTLTHGQGGDALGIVSMVNYTLDKHSGDSSRVYAMGFSSGGMMTNQLAGSYPDVFEAGAVYSGVAFGCAAGAESATPFSPNQTCAQGLQKTAQEWGDFVRNAYAGYTGRRPRMQIFHGLEDTLVRPQCAEEALKQWSNVLGVELTQEVSGVPSPGWTQKIYGDGTQLQGFFGQGIGHQSTVNEQQLLQWFGLI

SEQ ID NO: 91 - DNA, 1316 bp, Myceliophthora thermophila
atgaagtccttcaccctcaccactctggccgccctggctggcaacgccgccgctcacgcgaccttccaggccctctgggtcgacggcgtcgactacggcgcgcagtgtgcccgtctgcccgcgtccaactcgccggtcaccgacgtgacctccaacgcgatccgctgcaacgccaacccctcgcccgctcggggcaagtgcccggtcaaggccggctcgaccgttacggtcgagatgcatcaggtacgttggatgaatgagagaggaaagaaagcagaagcagaaggggaagggaaagaaaaagaaaaagaaaagaaaaagagaaagaaaaagaaagtggaaaccgtcggactaactgggctcttcttccccccctttgatatcagcaacccggtgaccgctcgtgcagcagcgaggcgatcggcggggcgcactacggccccgtgatggtgtacatgtccaaggtgtcggacgcggcgtcggcggacgggtcgtcgggctggttcaaggtgttcgaggacggctgggccaagaacccgtccggcgggtcgggcgacgacgactactggggcaccaaggacctgaactcgtgctgcgggaagatgaacgtcaagatccccgccgacctgccctcgggcgactacctgctccgggccgaggccctcgcgctgcacacggccggcagcgcgggcggcgcccagttctacatgacctgctaccagctcaccgtgaccggctccggcagcgccagcccgcccaccgtctccttcccgggcgcctacaaggccaccgacccgggcatcctcgtcaacatccacgccccgctgtccggctacaccgtgcccggcccggccgtctactcgggcggctccaccaagaaggccggcagcgcctgcaccggctgcgagtccacttgcgccgtcggctccggccccaccgccaccgtctcccagtcgcccggttccaccgccacctcggcccccggcggcggcggcggctgcaccgtccagaagtaccagcagtgcggcggccagggctacaccggctgcaccaactgcgcggtacgtttttttcttcagcccttttttcccgtctcctcttattatttattccctctggatttcttccttgccttctctgtccatacctctctgcatacatagggagcctagagacgacccctaccctacatctccctatcttatctggttacctaattacttacctcctcaatgtttctggctgctgactttttgctttagtccggctccacctgcagcgcggtctcgccgccctactactcgcagtgcgtctaa

SEQ ID NO: 92 - protein, 323 aa, Myceliophthora thermophila
MKSFTLTTLAALAGNAAAHATFQALWVDGVDYGAQCARLPASNSPVTDVTSNAIRCNANPSPARGKCPVKAGSTVTVEMHQQPGDRSCSSEAIGGAHYGPVMVYMSKVSDAASADGSSGWFKVFEDGWAKNPSGGSGDDDYWGTKDLNSCCGKMNVKIPADLPSGDYLLRAEALALHTAGSAGGAQFYMTCYQLTVTGSGSASPPTVSFPGAYKATDPGILVNIHAPLSGYTVPGPAVYSGGSTKKAGSACTGCESTCAVGSGPTATVSQSPGSTATSAPGGGGGCTVQKYQQCGGQGYTGCTNCASGSTCSAVSPPYYSQCV

SEQ ID NO: 93 - protein, 305 aa, Myceliophthora thermophila
HATFQALWVDGVDYGAQCARLPASNSPVTDVTSNAIRCNANPSPARGKCPVKAGSTVTVEMHQQPGDRSCSSEAIGGAHYGPVMVYMSKVSDAASADGSSGWFKVFEDGWAKNPSGGSGDDDYWGTKDLNSCCGKMNVKIPADLPSGDYLLRAEALALHTAGSAGGAQFYMTCYQLTVTGSGSASPPTVSFPGAYKATDPGILVNIHAPLSGYTVPGPAVYSGGSTKKAGSACTGCESTCAVGSGPTATVSQSPGSTATSAPGGGGGCTVQKYQQCGGQGYTGCTNCASGSTCSAVSPPYYSQCV